(19) United States
(12) Patent Application Publication
(10) Pub. No.: US 2026/0092261 A1
(43) Pub. Date: Apr. 2, 2026

(54) SIMULTANEOUS PRODUCTION OF STRUCTURAL PROTEINS FROM HETEROLOGOUS BACTERIOPHAGE IN CELL-FREE EXPRESSION SYSTEM

(71) Applicants: Elisabeth FALGENHAUER, Munich (DE); TECHNISCHE UNIVERSITÄT MÜNCHEN, Munich (DE)

(72) Inventors: Friedrich SIMMEL, Munich (DE); Kilian VOGELE, Raisting (DE); Sophie VON SCHÖNBERG, Eching (DE)

(73) Assignees: Elisabeth FALGENHAUER, Munich (DE); TECHNISCHE UNIVERSITÄT MÜNCHEN, Munich (DE)

(21) Appl. No.: 19/113,612

(22) PCT Filed: Sep. 20, 2023
(86) PCT No.: PCT/EP2023/075955

§ 371 Date: Mar. 20, 2025

(30) Foreign Application Priority Data
Sep. 20, 2022 (EP) 22196655.9

(51) Int. Cl. C12N 7/00; A61K 38/00; A61P 31/04; C07K 14/005

(52) U.S. Cl. CPC C12N 7/00; A61K 38/00; A61P 31/04; C07K 14/005; C12N 2795/00021; C12N 2795/00022; C12N 2795/00032; C12N 2795/00033; C12N 2795/00051

(57) ABSTRACT

The present invention relates to multi-peptide structures comprising at least one heterogenous functional site wherein the at least one heterogenous functional site is composed of at least two homologous peptides, which differ by at least one amino acid, a method for providing such multi-peptide structures, compositions comprising such multi-peptide structures as well as the use of such multi-peptide structures and compositions as an universal anti-microbial agent, in particular in medicine, chemistry, biotechnology, agriculture and/or food industry.

The present invention relates to multi-peptide structures comprising at least one heterogenous functional site wherein the at least one heterogenous functional site is composed of at least two homologous peptides, which differ by at least one amino acid, a method for providing such multi-peptide structures, compositions comprising such multi-peptide structures as well as the use of such multi-peptide structures and compositions as an universal anti-microbial agent, in particular in medicine, chemistry, biotechnology, agriculture and/or food industry.

Bacteriophages (herein also called “phages”) are viruses that specifically infect a host bacterium and proliferate at the expense of this host. They are composed of proteins that encapsulate a DNA or RNA molecule. Within their lytic state, Bacteriophages replicate within a host bacterium by injecting their viral genetic material into the host cell effectively taking over the cells functions for the production of progeny bacteriophage which leads to the rupture of the cell wall and subsequent bacterial cell death.

The biotechnological fields of application of bacteriophages are very broad and extend from evolution-based selection methods, like the evolutionary improvement of the activity of enzymes and other proteins, to phage display by which biological drug substances, e.g. therapeutic antibodies, could be generated and optimized, as well as the application of the bacteriophages themselves as a substitute for antibiotics.

The later use is based on the natural characteristics of bacteriophages to specifically affect bacteria, in particular pathogenic bacteria, and kill them, usually by lysing the bacteria or by inhibiting the replication system of the bacteria.

This approach that has been applied for a longer time to fight microbial infections and gets more and more attention as the number and spread of multi-drug resistant bacteria strains increases strongly worldwide. Methicillin-resistant Staphylococcus aureus (MRSA) bacteria, for example, is an increasingly common form of infection, often acquired through transmission in hospitals. MRSA infections are extremely difficult to treat using conventional antibiotics. The development of novel antibiotics is significantly slower than this development.

The development of phage-based therapeutics and diagnostics is, however, prevented by problems in the production and/or modification of bacteriophages. The complexity of the modification of bacteriophages is mainly due to the difficulty of changing the genome of bacteriophages.

These two main aspects of the production of bacteriophages, i.e. complexity of modification as well as simple and safe provision, have both been addressed at the same time by the methods and multi-peptide structures, i.e. synthetic bacteriophages, according to the present invention.

A first aspect of the present invention relates to a multi-peptide structure comprising at least one heterogenous functional site wherein the at least one heterogenous functional site is composed of at least two homologous peptides, which differ by at least one amino acid.

As understood herein, a “homogenous functional site” is composed of peptides, in particular non-engineered peptides of the same type of bacteriophage, whereas a “heterogenous functional site” is composed of homologous peptides differing by at least one amino acid, preferably derived from different bacteriophages.

The terms “peptide” and “protein” are understood by the person skilled in the art and may be used interchangeable where appropriate.

The term “homologous” as used herein is understood by the person skilled in the art and relates to proteins which are similar in position and structure and possibly evolutionary origin but not necessarily function. For example, the at least two homologous proteins comprised by the inventive multi-peptide structure show preferably a similar or almost identical structure. At least the structural elements necessary for assembly and/or interaction with other multi-peptide structure peptides are similar, preferably identical. Within a bacteriophage structure, the two homologous proteins are arranged in the same functional site as herein described. However, the at least two homologous proteins may differ in function such as receptor/host binding and/or specificity.

The inventive multi-peptide structure comprises at least one functional site wherein the at least one functional site being composed of at least two homologous peptides.

A “functional site” as herein understood denotes a component and/or a spatial position of peptides in the multi-peptide structure which fulfills a given function.

Generally, such functional sites as herein understood may be set up by single monomeric peptides or may be composed of a couple proteins, which may be identical or not, which assemble and/or bind together to form a structure like a capsid, tail or tail fiber. Thus, functional sites may be composed by monomeric peptides, oligomeric peptides as well as several different proteins. For example, capsid structures may be set up of several identical monomeric peptides, T7 tail fiber are set up as a homotrimer of three identical peptides and the T4 long fiber tail is set up of different proteins.

The multi-peptide structure can comprise several different functional sites. In general, a “functional site” according to the present invention may be selected, for example, from head (capsid), tail, spike, sheath, tube, baseplate and tail fiber components of a bacteriophage. Capsid and tail fiber functional sites are in particular preferred.

The inventive multi-peptide structure can contain a given functional site, i.e. a type of functional site, several times, e.g. one to six times, such as a fiber tail. For example, if the functional site comprising homologous proteins is a bacteriophage head or capsid component, there is one corresponding functional site in the multi-peptide structure; if the functional site comprising homologous proteins is a tail fiber there are usually one to six, preferably six, corresponding functional sites in the multi-peptide structure. For example, according to one embodiment, the inventive multi-peptide structure may comprise a given type of functional site only one time, e.g. like a capsid, which means that the at least two homologous proteins are both present within this functional site.

According to one embodiment, the multi-peptide structure may comprise a given type of functional site at least two times, e.g. like a fiber tail. In such an embodiment the at least two homologous peptides may be present at the same functional site position, if such functional site is set up by more than one protein, and/or may be present at the different same type functional site positions, in particular if the functional site is set up by one protein only.

“Homologous proteins” as used in the context herein refers to proteins which are not identical, i.e. which differ by at least one amino acid, i.e. do not have the same amino acid sequence” but fulfil essentially the same functional and/or structural role, e.g. have essentially the same specificity to other bacteriophage proteins to be assembled with, preferably self-assembled, in the inventive multi-peptide structure as defined above. Homologous peptides may show 80% or more sequence identity, preferably 85% or more, 90% or more or even 95% or more. Homologous peptides may differ by 1-30, or 1-25 or 1-20 or 1-15, or 1-10 or 1-5 amino acids. “Sequence identity” as herein understood refers to the occurrence of exactly the same amino acid in the same position in aligned sequences over the entire length. The homologous proteins can show conservative amino acid substitutions.

Their specificity of homologous proteins for targets, such as cell membranes or cell receptors, can be different.

According to a preferred embodiment the at least two homologous bacteriophage peptides are selected from the group comprising tail fiber proteins or tail fiber loops and/or capsid proteins, in particular receptor binding proteins. A tail fiber may be composed of homologous proteins including receptor binding proteins. For example, the long tail fiber of E. coli phage T4 is composed of four different proteins, wherein the distal subunit of the fiber, gp37, mediates receptor binding with its carboxy-terminal region.

The at least two homologous peptides, preferably derived from different bacteriophages, may be part of the same functional site in the multi-peptide structure, preferably a synthetic bacteriophage, which means that these two homologous bacteriophage peptides are both, for example, tail fiber proteins, tail fiber loops, capsid proteins, receptor binding proteins, etc.

According to a preferred embodiment, the multi-peptide structure resembles a bacteriophage structure; in other words, a multi-peptide structure according to the invention is preferably a “synthetic bacteriophage” or “engineered bacteriophage”.

In general, the basic structure of bacteriophages consists of a core of nucleus material, i.e. a nucleic acid, preferably genome, surrounded by a protein capsid (head).

Bacteriophages may exist in three basic structural forms, an icosahedral-like head with a tail, an icosahedral-like head without a tail and a filamentous form. Basic structures comprising an icosahedral head with a tail are preferred. Such a structure is shown in FIG. 1, for instance.

However, according to another preferred embodiment, the head may be deleted and/or the remaining bacteria penetration structure be altered. As understood herein such a “headless bacteriophage” is also comprised by the term synthetic bacteriophage. In particular, such “headless bacteriophages” can induce cell death without replication of the bacteriophage.

Despite the enormous differences in the shape of viruses, generalized principles for the structure of the protein coat can be found, since it is based on the self-assembly of smaller asymmetric building blocks.

For example, the general structure of most capsids is a polyhedron. This capsid can be constructed by applying symmetry principles with a small number of genes.

In order to arrange the asymmetric building blocks to form a symmetric structure, three of the building blocks must be placed in a triangular shape to form an icosahedron. In such a triangle, the building blocks have six equivalent contact points AE, three more equivalent contact points BC, and three similar contact points DD, as shown in FIG. 2.

Since the same contact points are used multiple times, a symmetric structure can be built from these triangular lattices. An icosahedron can be constructed from 20 equilateral triangles with 12 vertices. Since a triangle consists of 3 building blocks, the smallest possible icosahedron consists of 60 subunits. To increase the size of the icosahedral capsid, new smaller triangles must be built into the original triangles of the icosahedron. Since the structure must be symmetrical, only a certain number of new triangles can be added to the icosahedral structure. These newly added triangles form hexamers, while pentamers are formed at the vertices.

The number of newly formed hexamers H can be calculated using the triangulation number T, which indicates how many times the original triangle of the icosahedron is divided. The formula for this is H=10·(T−1). The triangulation number T can be calculated using two variables h≥1 and k≥0, which must be integers. h is the number of steps in a straight line from one pentamer to the nearest point of the adjacent pentamer, while k is the number of steps required in a straight line from the point taken with h steps to the adjacent pentamer, at a 60° angle from it. Thus, the triangulation number T itself is calculated via:

T=h²+h·k+k²

There are several examples of such icosahedral structures in viruses, such as rhinovirus and the virus family Parvoviridae with T=1, hepatitis B virus with T=3, and papillomaviruses and polyomaviruses with T=7 as well as with regard to the head of bacteriophages Escherichia-phage T7 with T=7, Enterobakteriophage MS2 with T=3 or Escherichia-phage T5 with T=13.

Considering such symmetry rules and considerations, capsid proteins can self-assemble into a capsid structure. Similar considerations apply to the multi-peptide structure of the present invention as well as other components of synthetic bacteriophages such as fiber tails and the tail itself.

According to a preferred embodiment, the multi-peptide structure comprises at least two homologous tail fiber proteins, preferably derived from different bacteriophages and/or at least two homologous capsid proteins, preferably derived from different bacteriophages. Such an embodiment may be preferably provided by a cell-free expression system as described below. A multi-peptide structure comprising at least two homologous tail fiber proteins is in particular preferred.

According to a preferred embodiment the multi-peptide structure is self-assembled. “Self-assembled” within the present invention refers to a multi-peptide structure, in particular synthetic bacteriophage, that has been spontaneously formed to a structural organization or pattern by the specific interaction of involved proteins. Such structural organization may be a synthetic bacteriophage comprising all components of a wild-type bacteriophage, or a synthetic bacteriophage not comprising all components of a wild-type bacteriophage such as a headless bacteriophage. The proteins providing a multi-peptide structure as herein described self-assemble via specific non-covalent interaction, i.e. the proteins providing a self-assembled multi-peptide structure are non-covalently bonded to each other.

The inventive multi-peptide structure is preferably capable of binding on the surface of a cell, in particular a bacterial cell. The homologous tail fiber proteins may be specific for the same or different hosts, wherein being specific for different hosts is preferred. The inventive multi-peptide structure or synthetic bacteriophage may be called multi-functional. “Host” as used herein describes the whole breadth of organisms comprising single species as well as strains which can be effected by the inventive multi-peptide structure or synthetic bacteriophage.

For example, homologous tail fiber proteins may be specific for gram positive and/or gram negative bacteria, in particular gram positive and gram negative bacteria. Having homologous tail fiber proteins and/or receptor binding proteins incorporated the host range of the inventive multi-peptide, in particular synthetic bacteriophage range, may be enhanced.

The homologous peptides may be derived from different bacteriophages, in particular different wild-type bacteriophages. However, they may be also derived from the same bacteriophage, wherein at least one peptide has been engineered.

According to another preferred embodiment all different homologous peptides are engineered. According to yet another embodiment, the bacteriophages are derived from the same wild-type but engineered in a different way.

The different bacteriophages where the homologous peptides are derived from, in particular wild-type bacteriophages, may be selected from the group comprising Caudovirales (family of Myoviridae, Autographiviridae, Podoviridae, Ackermannviridae, and Siphoviridae), Leviviridae, Tectiviridae, Corticoviridae, Plasmaviridae, Sphaerolipoviridae, Inoviridae, Microviridae, Picobirnaviridae and/or Cystoviridae.

According to a preferred embodiment, the inventive multi-peptide structure comprises a bacteriophage derived nucleic acid, in particular bacteriophage genome, encoding at least one homologous peptide less than comprised by the inventive multi-peptide structure; i.e. after replication, the 1. generation multi-peptide structure, in particular synthetic bacteriophage, differs from the parent multi-peptide structure as the 1. generation multi-peptide structure comprises at least one homologous peptide, being not encoded, less than the claimed multi-peptide structure.

Such bacteriophage derived nucleic acid may be any genetic material or nuclear material of bacteriophages, in particular bacteriophage a genome, which can be either DNA or RNA and/or variants thereof, which can either be double-stranded or single-stranded. Such genetic material or nuclear material of bacteriophages, in particular of a bacteriophage genome, may also be engineered. However, according another embodiment such bacteriophage derived nucleic acid is a unmodified bacteriophage genome, in particular wild-type genome. According to certain embodiments, the genetic material facilitates the replication of the multi-peptide structure, in particular of the synthetic bacteriophage.

Engineering, e.g. peptide engineering, may expand the abilities of the underlying protein by approaches known to the person skilled in the art und includes in particular site-specific mutations such as insertions, deletions and/or point mutations of the corresponding protein encoding nucleic acids, incorporation of non-natural amino acids as well as the addition of further functional groups such as labeling groups, active agents etc. As understood by the person skilled in the art such engineering can be performed on the nucleic acid level as well as on the protein level.

Due to the degeneracy of the genetic code, any nucleic acid sequence can be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. Such a conservative change may be, for example, done for improved expression of the protein in a given expression system.

According to a preferred embodiment, at least one of the receptor binding proteins and/or capsid forming proteins comprises non-naturally generated mutations, e.g. resulting from deletions, insertions and/or substitutions within the corresponding encoding nucleic acid as described above.

According to a preferred embodiment the multi-peptide structure, in particular synthetic bacteriophage, may be labelled. Such a label may be, for example, a marker, or an active agent. A “marker” as understood herein may be a tag such as biotin, a his-tag or any other tag recognizable by a binding partner such as an antibody, useable, for example, to isolate the bacteriophage and/or tags masking bacteriophages from the immune system of an object to be treated, such as peptides of human cells to mask the bacteriophage from the human immune system. Markers suitable for use in affinity purification processes include glutathione-5-transferase (GST), protein A, ScFv and lectins. A marker may be also a dye or a fluorescent label, e.g. for use in detection assays. Other modifications of the bacteriophage may be made, e.g. for reducing bacteriophage antigenicity, including use of a PEG (polyethyleneglycol) conjugate or a polysialic acid conjugate. An “active agent” as understood herein enhance the lethality of the bacteriophage to the bacterial host. The label may be incorporated at the DNA level, may be attached chemically at the phage surface or may even self-assemble into the inventive multi-peptide structure, in particular synthetic bacteriophage, within the inventive method described herein.

Such a label, marker and/or tag may be added to any peptide of the inventive multi-peptide structure.

Another aspect of the invention relates to a method, in particular in vitro method, for providing multi-peptide structures, in particular synthetic bacteriophages, comprising the steps of
(a) providing an expression system, in particular a cell-free expression system,
(b) adding nucleic acids encoding homologous proteins, preferably of at least two different bacteriophages, in particular homologous phage tail fiber proteins and/or homologous phage capsid proteins, to the expression system,
(c) expressing the nucleic acids encoding the homologous proteins, preferably of the at least two different bacteriophages, in particular homologous phage tail fiber proteins and/or homologous phage capsid proteins, preferably of different bacteriophages,
(d) assembling of the expressed homologous proteins, preferably of the at least two bacteriophages, in particular homologous phage tail fiber proteins and/or homologous phage capsid proteins, preferably of different bacteriophages, to provide the assembled multi-peptide structures, in particular the synthetic bacteriophages, and optional
(e) isolating of the assembled multi-peptide structures, in particular the synthetic bacteriophages.

According to an especially preferred embodiment, the inventive method is an “all in vitro method”. According to the present invention, this means that neither for replication of nucleic acids, in particular synthetic bacteriophages, nor the expression of such nucleic acids as well as the assembly of the corresponding proteins any cell, virus or bacteriophage based system is necessary.

The nucleic acids encoding proteins of at least two different viruses according to step (b) may be added in a predetermined ratio to determine the composition of the assembled multi-protein structure, in particular synthetic bacteriophage, to be provided. Such nucleic acids may be DNA and/or RNA molecules, such as PCR products, plasmid vectors, native DNA and/or RNA molecules, chemically or biologically synthesized molecules, artificial chromosomes, in particular yeast artificial chromosomes and/or bacterial artificial chromosomes, etc. According to a preferred embodiment, the DNA and/or RNA molecules are essentially complete genomes of different bacteriophages.

The at least two different bacteriophages referred to in step (b) may be at least two different wild-type bacteriophages, a first wild-type bacteriophage and at least one mutated or engineered bacteriophage derived from said first wild-type bacteriophage or at least two mutated or engineered bacteriophages derived from the same wild-type bacteriophage. By varying the concentration of the added coding genetic information, it is possible to adjust the final concentration of the resulting multi-peptide structure for further use.

At least one of the protein encoding nucleic acids may be modified, in particular modified by non-naturally mutations such as deletion, insertion and/or substitution as described herein above.

At least one of the homologous proteins is preferably a capsid protein and/or tail fiber protein.

Of course, the multi-peptide structures, in particular bacteriophages, provided herein are preferably lysis-efficient, i.e. capable of lysing their target organism or host.

The multi-peptide structures, in particular bacteriophages are preferably provided in an isolated form. Corresponding techniques for isolating or purifying are known to the person skilled in the art.

The cell-free expression system is a preferably host independent system. The term “cell free” is understood by the person skilled in the art and refers to “substantially free of”.

A “cell-free expression system” as understood herein comprises a complete transcription and translation machinery for transcription and expression of a virus or bacteriophage nucleic acid, in particular a bacteriophage genome.

A preferred example for a cell free expression system is a cell lysate. Using such a cell lysate it is possible to synthesize several proteins or metabolites at the same time.

“Cell lysate” according to the present invention refers to a fluid comprising the components of cells from which it is derived after lysis. Lysis methods are known to the person skilled in the art and break down the membrane of a cell, for example, by viral, enzymatic, or osmotic mechanisms that compromise its integrity. Such cell lysate is essentially void of intact cells, i.e. cell-free. The terms “cell lysate” and “cell extract” can be used herein interchangeably.

After purification this lysate is essentially free, preferably free, of host DNA and makes an expression of the desired protein possible by the external addition of DNA, in particular a amplified bacteriophage genome as herein described. Thus, the used cell lysate is preferably free of nucleic acids, in particular DNA, and/or membranes from the cells of which it is derived. In a preferred embodiment, using standard techniques no host DNA can be detected in the purified lysate.

Cell lysates used according to the present invention may be derived from microorganisms, in particular bacteria such as pathogenic bacteria, E. coli, yeast, insects, mammals and/or plants, in particular such as wheat or rice, or may be even artificial. “Microorganism” refers to a bacterium or an archaeon. Preferably, the microorganism is a bacterium.

A number of cell-free expression systems is known to a person skilled in the art und may be applied according to the present invention. For example, the “PURE” system consists of several isolated proteins (Shimizu et al.) while untreated cell lysates (crude extracts) such as of E. coli include almost all intracellular proteins, even those that are not necessary for expression.

The use of E. coli lysate, in particular crude E. coli lysate, is a further preferred embodiment. A preferred example of crude E. coli lysate is E. coli S30 cell extract produced by a method based on the protocol described in E. Falgenhauer et al. (Falgenhauer, S. von Schönberg, C. Meng, A. Mückl, K. Vogele, Q. Emslander, C. Ludwig, F. C. Simmel, ChemBioChem 2021, 22, 2805).

External factors such as energy carriers, co-factors, amino acids, polymerases, transcription regulatory factors, chaperons, DNA stabilization agents (e.g. GamS and/or Chi6 DNA) may be added to enhance or otherwise improve the reaction. Such factors may be added as proteins and/or as nucleic acid encoding a respective protein.

According to a preferred embodiment, polyethylene glycol (PEG) may be added to the cell extract to increase molecular crowding. PEG binds water, creating a more concentrated environment. This may be advantageous because there is up to 25 to 30 times less protein concentration in the cell extract than in the cytosol of a bacterial cell.

If the cell-free lysate to be used is derived from a cell or microorganism which is different to the natural host of the virus or bacteriophage, at least one bacteriophage-host specific expression factor and/or a nucleic acid encoding the at least one bacteriophage-host specific expression factor, preferably a transcription factor, can be added, if necessary. “Bacteriophage specific host factor” or “bacteriophage-host specific expression factor” with regard to a given bacteriophage are well known to the person skilled in the art and/or can be easily determined (cf e.g. Role of host factors in bacteriophage φ29 DNA replication, Adv Virus Res 2012; 82:351-83 or US2022025957A1)

According to one embodiment of the invention, at least one nucleotide sequence encoding the at least one bacteriophage-host specific expression factor or any other protein preferably selected from the group comprising co-factors, chaperons, polymerases, transcription regulatory factors is cloned into the cell strain used to provide the cell lysate. The nucleotide sequence is expressed within the cell before lysis. Thus, after lysis the cell lysate already contains all nucleic acids and/or proteins necessary for the amplification of the virus or bacteriophage, in particular at least one host specific expression factor or any other protein selected from the group comprising co-factors, chaperons, polymerases, transcription regulatory factors and/or any mixture thereof.

Further modifications of the synthetic bacteriophage to be provided can be achieved by adding a modified protein to the cell-free expression system and/or by adding a nucleic acid encoding that modified protein. This protein itself can be of natural amino acid sequence or artificially modified for optimization.

The cell-free synthesis of proteins, i.e. outside living cells, has several advantages over cellular expression. This is all the more true if proteins are expressed that are toxic for the bacteria or non-natural amino acids shall be inserted into the proteins.

The production of bacteriophages in a cell-free lysate may also by carried out without any bacteriophage host bacteria, which makes it possible to produce them locally, e.g. in a laboratory without biosafety-level. At the same time the amount of toxic by-products that are produced by a method according to the present invention is reduced, since lysed pathogenic host bacteria are not present.

Another advantage is that if the in vitro method is based on a cell-free lysate only the genome of the added virus or bacteriophage is applied and thus, a pure solution of viruses or bacteriophage can be produced, without any pro-viruses or pro-phages. The cell-free expression in combination with the in vitro reproduction of nucleic acids and in particular the genome represents a broadly applicable method that accelerates the application of bacteriophages in research and development and biomedical applications.

During assembly step (d) the expressed proteins preferably self assemble to the inventive multi-peptide structure, in particular synthetic bacteriophage. Self-assembly can be supported and/or initiated by the addition of supporting factors, e.g. PEG, certain buffer systems e.g. standard phosphate buffered saline und in particular ions. Exemplary ions include, but are not limited to, Li⁺, Na⁺, K⁺ and Cs⁺ as well as divalent ions such as Ca²⁺ or Mg²⁺. In certain embodiments, the concentration of the monovalent and/or divalent ions is at least 5 mM, 10 mM, 20 mM or 50 mM in order to induce or substantially accelerate self-assembly. Self-assembled multi-peptide structures can be provided with high reproducibility and uniformity.

The inventive method enables the provision of a composition comprising different synthetic bacteriophages having a statistical combination of the functional sites described herein, such as different tail fiber assemblies. The combination of, for example, homologous tail fiber proteins during the assembly of the expressed proteins during step (d) at a functional site as described herein entails a combinatorial expansion of the possible binding sites of the provided composition comprising different synthetic bacteriophages due to the possibility of the different structure of the tail fiber. According to the invention, this combinatorial expansion is achieved in a simple manner by self-assembly at the protein level.

The concentration of multi-peptide structures, in particular bacteriophages, generated in vitro may be so high that the corresponding composition and/or formulation can directly be applied in a therapy with phages without a further concentration step. If needed, a further concentration step, in particular after purification, is of course also within the scope of the present invention.

A further aspect of the invention relates to a multi-peptide structure, in particular a synthetic bacteriophage, provided by the methods described herein. All embodiments described herein relate preferably to self-assembled multi-peptide structures, in particular synthetic bacteriophages.

Another aspect of the invention relates to a composition comprising the multi-peptide structure, in particular synthetic bacteriophage, as described herein.

A further aspect of the invention relates to a composition comprising two or more types of multi-peptide structures, in particular synthetic bacteriophages, as described herein, wherein the two or more types of the multi-peptide structure, in particular synthetic bacteriophage, have homologous tail fiber and/or capsid proteins or have different mutations in the tail fiber and/or capsid proteins. Such a composition comprises different synthetic bacteriophages which may have a statistical combination of the functional sites set up by the at least two homologous peptide, such as a statistical combination of tail fiber with regard to their protein composition as well as their positioning in the multi-peptide structure.

It is preferred that the two or more types of the multi-peptide structure, in particular synthetic bacteriophage, have different host ranges.

The inventive compositions may comprise carriers and/or vehicles as described below. According to preferred embodiments, the compositions are pharmaceutical compositions.

A further aspect of the invention relates to the multi-peptide structure, in particular synthetic bacteriophage, provided as described herein for use in medicine, i.e. as a medicament, chemistry, biotechnology, agriculture and/or food industry. Such uses are not limited to bacteria related fields and comprise, for example, the use as vaccine, for example against infections as well as tumors, and the use as a delivery vehicle for any kind of drug, in particular anti-cancer drugs, to a target cell, in particular a human target cell. A preferred embodiment relates to the use of a multi-peptide structure, in particular synthetic bacteriophage, provided as described herein for use as a medicament.

According to preferred embodiments, the multi-peptide structures, in particular synthetic bacteriophages, as well as compositions described herein are used to infect targets, in particular hosts, in a first infection cycle. Thereby it may be avoided that the multi-peptide structures, in particular synthetic bacteriophages, revert to their original non-engineered state after one round of infection.

The use in bacteria related fields is, however, preferred. Multi-peptide structures, in particular synthetic bacteriophages, may in particular be used as bacterial population control alternatives to antibiotics. They are much more specific than antibiotics and are typically harmless not only to the host organism but also to other beneficial bacteria, such as the gut microbiota, as they are very selective in the strains of bacteria, they are effective against. Thereby the chances of opportunistic infections are significantly reduced. Advantages include further reduced side-effects and reduced risk of the bacterium's developing resistance. Because phages replicate in vivo, in certain embodiments small effective doses can be used. These unique properties make them highly promising antimicrobials.

Accordingly, the multi-peptide structures, in particular synthetic bacteriophages, provided herein may be part of a pharmaceutical composition. Such a composition may comprise (i) at least one bacteriophage strain capable of producing a lytic infection and (ii) a pharmaceutically acceptable carrier and/or vehicle. Of course, such composition may comprise synthetic bacteriophage mixtures. A preferred embodiment relates to such a composition for use as a medicament.

The pharmaceutical compositions may be used in combination with antibiotics for the purpose of treating bacterial infections and/or to treat antibiotic resistant bacteria. In particular, bacteriophages tend to be more successful than antibiotics where there is a biofilm covered by a polysaccharide layer, which antibiotics typically cannot penetrate. According to a preferred embodiment, the composition may be freeze dried.

“Pharmaceutically acceptable carrier” relates to pharmaceutically-acceptable, fillers or diluents used to formulate pharmaceutical compositions for animal or human administration. The pharmaceutical compositions may further comprise pharmaceutically acceptable auxiliary agents, and optionally other therapeutic agents. In particular for oral administration it is preferred to add an antacid, i.e. a substance which neutralizes stomach acidity, thereby increasing the number of phages surviving passage through the stomach.

“Vehicle” as used herein refers to any compound or combination known to the person skilled in the art known to be useful in formulating a composition, in particular a pharmaceutical composition. Such a vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material.

In tablets, the active agent (i.e. multi-peptide structure, in particular bacteriophage) may be mixed with a vehicle having the necessary compression properties in suitable proportions and compacted in the shape and size desired.

Suitable solid vehicles include, for example calcium phosphate, magnesium stearate, talc, sugars, lactose, dextrin, starch, gelatin, cellulose, polyvinylpyrrolidine, low melting waxes and ion exchange resins.

In another embodiment, the pharmaceutical vehicle may be a gel and the composition may be in the form of a cream or the like.

The dose and regimen of administration of a pharmaceutical composition will necessarily be dependent upon the therapeutic effect to be achieved (e.g. treatment of IBD) and may vary with the particular bacteriophage strains in the composition, the route of administration, and the age and condition of the individual subject to whom the pharmaceutical composition is to be administered.

If the pharmaceutical composition is to be administered with one or more antibiotics, it may be simultaneously, separately or sequentially administered.

Pharmaceutical compositions and routes of administration include those suitable for or via oral (including buccal, sublingual and intraorbital), rectal, nasal, topical (including transdermal), ocular, vaginal, bronchial, pulmonary or parenteral (including subcutaneous, intramuscular, intravenous, intradermal, intraperitoneal, intrapleural, intravesicular and intrathecal) administration or administration via an implant. In particular a freeze-dried composition may be administered orally, preferably on form of a pill.

Agriculture comprises plant agriculture as well as animal agriculture, wherein bacteriophages can replace or complement antibiotics and/or pesticides.

Bacterial pathogens are associated with a couple of plant diseases which can also be treated with synthetic bacteriophages provided herein. Non limiting examples for bacterial plant diseases which might be treated are leaf blight on onion, potato scab or soft rot of potato, bacterial wilt on tobacco, citrus bacterial spot or citrus canker of citrus, fire blight on apple, black rot of cabbage, soft rot of calla lilies, bacterial wilt of tomato and bacterial spot of peach.

The use of the inventive multi-peptide structures, in particular synthetic bacteriophages, in animal agriculture is exemplified by the use against Salmonella, pathogenic E. coli, Clostridium, and Campylobacter for the poultry industry and against Salmonella and pathogenic E. coli for the pig industry.

Multi-peptide structures, in particular synthetic bacteriophages, provided herein may also be used to safen food products, and to forestall spoilage bacteria. Since 2006, the United States Food and Drug Administration (FDA) and United States Department of Agriculture (USDA) have approved several bacteriophage products. For example, LMP-102 (Intralytix) was approved for treating ready-to-eat (RTE) poultry and meat products. In that same year, the FDA approved LISTEX using bacteriophages on cheese to kill Listeria monocytogenes bacteria.

With regard to biotechnology related uses, the methods described herein as well as multi-peptide structures, in particular synthetic bacteriophages, provided herein can, for example, be used for phage display related application.

The invention further includes a kit comprising a pharmaceutical composition of the invention and instructions for the use of the composition for a use as hereinbefore described, optionally together with packaging material.

Further aspects relate to the use of multi-peptide structures, in particular synthetic bacteriophages, provided herein in methods for the prevention and or treatment of a bacterial infection as well as for avoiding bacterial growth.

The present invention is in particular described by the following items:

1. A multi-peptide structure comprising at least one heterogenous functional site wherein the at least one heterogenous functional site being composed of at least two homologous peptides, which differ by at least one amino acid.

2. The multi-peptide structure according to item 1, wherein the at least two homologous peptides are derived from different bacteriophages and/or are engineered in a different way.

3. The multi-peptide structure according to item 1 or 2, comprising a bacteriophage derived nucleic acid, in particular bacteriophage genome, encoding at least one homologous peptide less than comprised by the multi-peptide structure.

4. The multi-peptide structure of any of the preceding items, wherein the functional site is selected from head (capsid), tail, spike, sheath, tube, baseplate or fiber component of a bacteriophage, preferably a capsid or fiber component and even more preferably a fiber tail component.

5. The multi-peptide structure of any of the preceding items, wherein the at least two homologous peptides are selected from the group comprising receptor binding proteins, tail fiber proteins or tail fiber loops, and/or capsid proteins.

6. The multi-peptide structure of any of the preceding items,
wherein the multi-peptide structure comprises at least two homologous bacteriophage tail fiber proteins and/or at least two homologous bacteriophage capsid proteins, preferably derived from different bacteriophages.

7. The multi-peptide structure of any of the preceding items,
wherein at least one of the at least two homologous bacteriophage peptides is engineered.

8. The multi-peptide structure of any of the preceding items,
wherein the multi-peptide structure is capable of binding on the surface of a cell, in particular a bacterial cell.

9. The multi-peptide structure of any of the preceding items,
wherein the homologous tail fiber proteins are specific for different hosts.

10. The multi-peptide structure of any of the preceding items,
wherein the homologous tail fiber proteins are specific for the same host.

11. The multi-peptide structure of any of the preceding items,
wherein the homologous tail fiber proteins are specific for gram positive and/or gram negative bacteria, in particular gram positive and gram negative bacteria.

12. The multi-peptide structure of any of the preceding items,
wherein the multi-peptide structure is assembled, preferably self-assembled, and/or resembles a headless phage.

11. The multi-peptide structure of any of the preceding items,
wherein the different bacteriophages are selected from different wild-type bacteriophages.

13. The multi-peptide structure of any of the preceding items,
wherein the different bacteriophages, in particular wild-type bacteriophages, are selected from the group comprising Caudovirales (family of Myoviridae, Autographiviridae, Podoviridae, Ackermannviridae, and Siphoviridae), Leviviridae, Tectiviridae, Corticoviridae, Plasmaviridae, Sphaerolipoviridae, Inoviridae, Microviridae, Picobirnaviridae and/or Cystoviridae.

15. The multi-peptide structure of any of the preceding items,
wherein at least one of the receptor binding proteins and/or capsid forming proteins comprises non-naturally generated mutations, e.g. resulting from deletions, insertions and/or substitutions within the corresponding encoding nucleic acid.

16. The multi-peptide structure of any of the preceding items,
wherein the multi-peptide structure is labelled.

17. Method, in particular in vitro method, for providing multi-peptide structures, in particular synthetic bacteriophages, comprising the steps of
(a) providing an expression system, in particular a cell-free expression system,
(b) adding nucleic acids encoding homologous bacteriophage proteins, preferably derived from different bacteriophages, in particular homologous bacteriophage tail fiber proteins and/or homologous bacteriophage capsid proteins, to the expression system,
(c) expressing the nucleic acids encoding the homologous proteins, in particular homologous phage tail fiber proteins and/or homologous phage capsid proteins,
(d) assembling of the expressed homologous proteins, in particular homologous phage tail fiber proteins and/or homologous phage capsid proteins, to provide the assembled multi-peptide structures, in particular the synthetic bacteriophages, and optional
(e) isolating of the assembled multi-peptide structures, in particular the synthetic bacteriophages.

18. The method of item 17,
wherein the expression system is a cell-free expression system.

19. The method of any of items 17 or 18,
wherein the cell-free expression system is host independent and preferably selected from cell lysates or artificial expression systems.

20. The method of any of items 17-19,
wherein the cell lysates are derived from microorganisms, in particular bacteria, yeast, insects, mammals, plants and/or are artificial.

21. The method of any of items 17-20,
comprising the addition of bacteriophage-host specific factors.

22. The method of any of items 17-21,
wherein in step (b) the nucleic acids encoding homologous proteins, preferably of at least two different bacteriophages, are added in a predetermined ratio to determine the composition of the assembled multi-peptide structure, in particular synthetic bacteriophage, to be provided.

23. The method of any of items 17-22,
wherein the nucleic acids encoding proteins of the at least two different bacteriophages are DNA and/or RNA molecules, such as PCR products, plasmid vectors, native DNA and/or RNA molecules, chemically or biologically synthesized molecules, artificial chromosomes, in particular yeast artificial chromosomes and/or bacterial artificial chromosomes, etc.

24. The method of any of items 17-23,
wherein the DNA and/or RNA molecules are essentially complete genomes of bacteriophages.

25. The method of any of items 17-24,
wherein the at least two different DNA and/or RNA molecules are one essentially complete genome of a bacteriophage, and at least one nucleic acid molecule encoding at least one selected homologous protein, in particular a tail fiber or capsid protein, derived from another bacteriophage.

26. The method of any of items 17-25,
wherein at least one of the homologous protein encoding nucleic acids is modified, in particular modified by non-naturally mutations such as deletion, insertion and/or substitution.

27. The method of any of items 17-26,
wherein at least one homologous protein derived from another bacteriophage, is a capsid protein and/or tail fiber protein.

28. The method of any of items 17-27,
wherein only one type of nucleic acid molecule, in particular an expression vector, encoding homologous proteins, preferably of at least two different bacteriophages, is added.

29. Multi-peptide structure, in particular a synthetic bacteriophage, provided by the method of any of items 17-29.

30. Composition comprising the multi-peptide structure, in particular synthetic bacteriophage, according to any of items 1-16 or 29.

31. Composition comprising two or more types of multi-peptide structure, in particular synthetic bacteriophages, according to any of items 1-16 or 29, wherein the two or more types of the multi-peptide structure, in particular synthetic bacteriophages, have homologous tail fiber and/or capsid proteins or have different mutations in the tail fiber and/or capsid proteins.

32. The composition of item 31, wherein the two or more types of the multi-peptide structure, in particular synthetic bacteriophage, have different host ranges.

33. An universal anti-microbial agent comprising the multi-peptide structure of any of items 1-16 or 29 or the composition according to any of items 28-30. The wordings “universal anti-microbial agent” and “anti-microbial agent” can be used interchangeably herein.

34. Multi-peptide structure, in particular a synthetic bacteriophage, according to any of items 1-16 or 29 or the composition according to any of items 28-30 for use in medicine, chemistry, biotechnology, agriculture and/or food industry.

35. The multi-peptide structure, in particular a synthetic bacteriophage, or composition according to item 34 for use in a method for the prevention and or treatment of a bacterial infection.

36. Use of a multi-peptide structure, in particular a synthetic bacteriophage, according to any of items 1-16 or 29 or composition according to any of items 30-32 for avoiding bacterial growth.

### FIGURES

FIG. 1: General schematic Figure of a T7 like bacteriophage with a icosahedral head, a tail and tail-fiber

FIG. 2: Illustration of the triangular lattice built from asymmetric building blocks to form an icosahedron-like structure. The possible contact points of the structure are marked with the letters A-E.

FIG. 3: Concentration (plaque forming units/ml (PFU/ml)) of different simultaneously expressed particles in cell extract plated on different hosts.

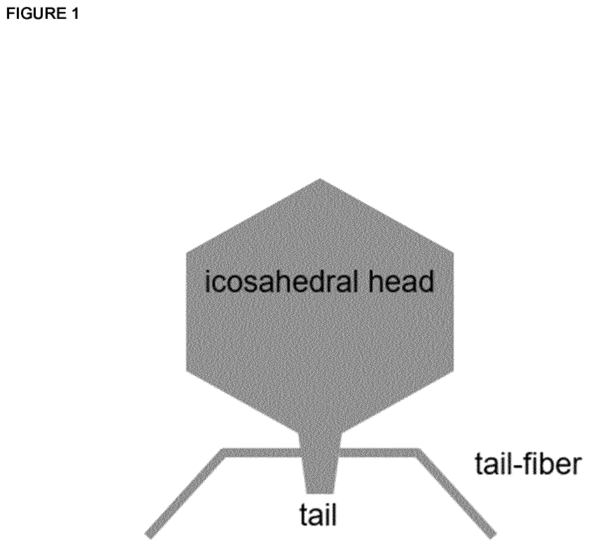

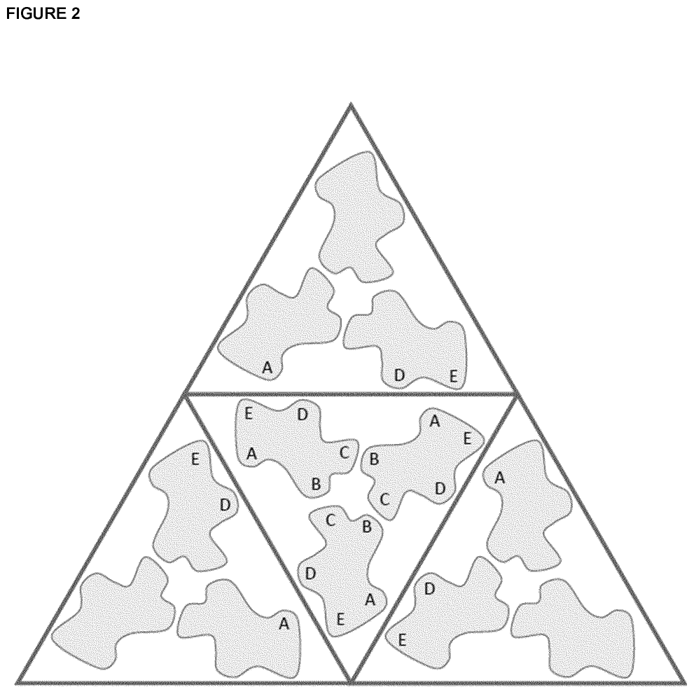

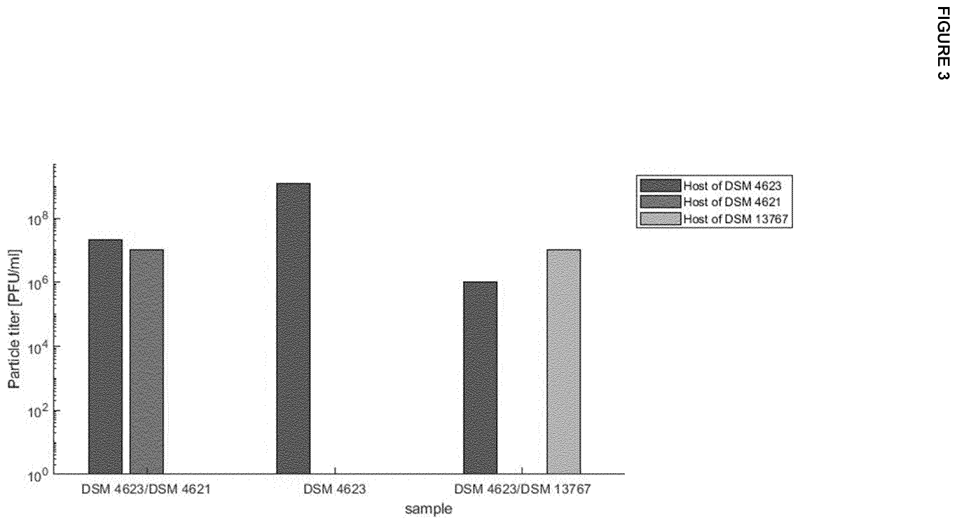

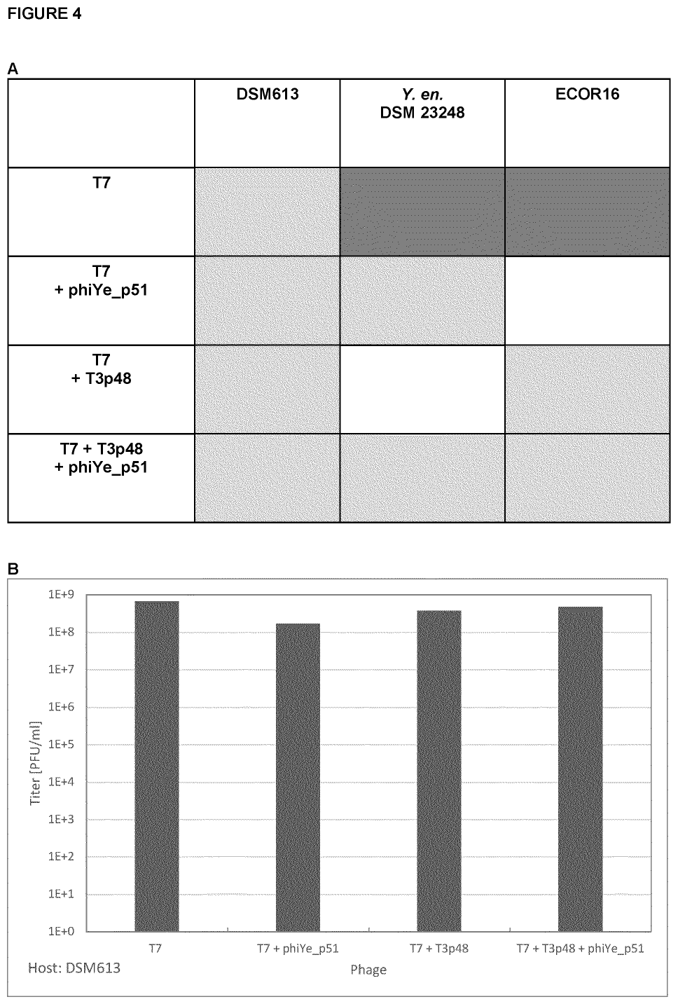

FIG. 4: Co-expression of Native T7 Phage with Tail-Fibers from different Phages.
(A) Spot assay analysis of the lysis capability of various phages.
The following combinations were tested: Cell-free expressed native T7 phage co-expressed with a plasmid encoding the phiYe_p51 tail fiber of the Yersinia phage phiYe03-12; Cell-free expressed native T7 phage co-expressed with a plasmid encoding the T3p48 tail-fiber protein; Cell-free expressed native T7 phage co-expressed with plasmids encoding both the T3p48 tail-fiber protein and the phiYe_p51 tail fiber of the Yersinia phage phiYeO3-12. A sample of the native T7 phage is also provided as a reference.
Light grey indicates samples where lysis was observed (visible lysis), while dark grey represents samples without lysis (no visible lysis). The respective hosts are: DSM613 for the T7 phage, DSM 613 and ECOR16 for the T3 phage, and Yersinia enterocolitica DSM 23248 for the phiYeO3-12 phage. Successful co-expression of T7 and phiYe_p51 suggests the system's potential to generate phages that can infect two different bacterial species and with the addition of the plasmid encoding the T3p48 tail-fiber protein also three different tail-fiber can be used.
(B) Titer results of the cell-free expressed phages on DSM613. Introducing alternate tail-fibers resulted in only a minor decrease in plating efficacy compared to the native T7 phage, also for the case with the three tail-fibers present in the reaction.

FIG. 5: Exploration of diverse tail fiber combinations on T7Δp52 phage. Here the T7Δp52 phage (T7 phage lacking the tail fiber gene p52) is used as chassis, with various non-T7 tail fibers together.
(A) Spot assay results for lysis capability of different tail fiber combinations: T7Δp52 phage with a plasmid encoding the T3p48 tail-fiber protein; T7Δp52 phage with a plasmid encoding the Bas65_p50 tail-fiber protein; T7Δp52 phage co-expressed with plasmids for both T3p48 and Bas65_p50 tail-fiber proteins.
Samples are marked in light grey if lysis (visible lysis) was observed, and in dark grey if no lysis (no visible lysis) was seen. The corresponding hosts are: DSM 613 and ECOR16 with the T3 phage (and its tail-fiber T3p48), DSM 613 and DSM 27469 with the Bas65 phage (and its tail-fiber Bas65_p50). Results demonstrate: T3p48 facilitates infection of DSM613 and ECOR16, Bas65_p50 enables infectivity towards E. coli DSM 27469 and DSM613, and combined tail fibers allow the phage to infect all three hosts.
(B) Titer results of cell-free expressed phages when grown on host DSM613+T7p52 to observe plaques. Different tail fiber co-expressions yield varying titers.

FIG. 6: Exploration of tail fiber combinations of native tail fibers and tail fibers with artificial point mutations on T7Δp52 Phage.
Using the T7Δp52 phage as a base, various tail fiber proteins—including native T7p52, its point mutations, and combinations thereof—were explored. Each point mutation represents an alanine-substitution situated in the C-terminal binding domain of T7p52. Plaque assays were performed on DSM613+T7p52 for each sample. The efficacy of plating (EOP) was then calculated using T7Δp52+wild-type T7p52 as a reference.
The bars represent: Dark grey bar, T7Δp52 co-expressed with a single tail-fiber mutant; Medium grey bar: T7Δp52 co-expressed with one tail-fiber mutant and the wild-type T7p52; Medium grey bar with ‘+’: T7Δp52 when co-expressed with the wild-type T7p52 and two different point mutation variants.
The expression of solely mutated T7p52 variants leads to a pronounced reduction in EOP, as shown by the dark grey bar. Introducing wild-type T7p52 with the mutated versions moderately restores phage infectivity, leading to a lesser reduction in EOP (medium grey). The tripartite co-expression, incorporating two point mutation variants alongside the wild-type T7p52, showcases combined phenotypic effects, represented by the medium grey bar with ‘+’.

FIG. 7: Exploration of Tail Fiber Combinations of artificial designed Tail-fiber. T7p52 (S543A) and native tail fibers of other phages on T7Δp52 Phage. Using T7Δp52 phage as a chassis, a set of tail fiber proteins, including T7p52 (S543A) and its combinations with native tail fibers from various phages, were studied.
(A) Spot assay analysis of the lysis capability of various phages. The following combinations were tested:
Cell-free expressed T7Δp52 phage without plasmid as a reference; Cell-free expressed T7Δp52 phage with a plasmid encoding T7p52 as a reference; Cell-free expressed T7Δp52 phage with a plasmid encoding T7p52 (S543A); Cell-free expressed T7Δp52 phage with a plasmid encoding T7p52 (S543A) with the native T7p52 on a plasmid; Cell-free expressed T7Δp52 phage with a plasmid encoding T7p52 (S543A) with the native T3p48 on a plasmid; Cell-free expressed T7Δp52 phage with a plasmid encoding T7p52 (S543A) with the native phiYe_p51 on a plasmid; Cell-free expressed T7Δp52 phage with a plasmid encoding T7p52 (S543A) with the native T3p48 and phiYe_p51 on a plasmid; Cell-free expressed T7Δp52 phage with a plasmid encoding T7p52 (S543A) with the native T7p52 and phiYe_p51 on a plasmid;
In samples marked in light grey, lysis was observed; samples in dark grey no lysis was observed.
(B) Titer of the cell-free expressed phages on DSM613+T7p52 to make plaques visible.
The point mutation T7p52 (S543A) shows a reduced EOP compared to the native tail fiber T7p52, the combination of T7p52 (S543A) and T7p52, shows an intermediate Phenotype. The addition of further (native) tail fibers show the same host range expansion as the combination of native tail fibers to T7p52 (wildtype), but with the reduced EOP conferred by T7p52 (S543A) Only the phage encoding for the tail-fiber targeting ECOR16 and and Y.en DSM 23248 were able to infect these bacteria.

FIG. 8: Expanding the host range of native T7 phage through co-expression with tail-fiber T7p52 variants featuring artificial tip modifications.
Utilizing the native T7 phage as a chassis, the co-expression with various modified versions of the T7p52 tail-fiber was explored. These modifications include loop exchanges and complete tip substitutions with regions from other phages.
The following combinations were tested:
(A) Cell-free expressed native T7 phage without plasmid as a reference; Cell-free expressed native T7 phage with a plasmid encoding T7p52 (Loop-T3p48), a T7p52 protein with exchange of 2 binding Loops with for the analogous region of T3p48; Cell-free expressed native T7 phage with a plasmid encoding T7p52 (Loop-phiYe_p51) a T7p52 protein with exchange of 2 binding Loops with for the analogous region of phiYe_p51; Cell-free expressed native T7 phage with a plasmid encoding T7p52 (Tip-phiYe_p51) a fusion protein of N-terminal part T7p52 [1-466] with C-terminal binding domain (137 AA) from phiYe_p51;
In light grey the samples with a visible lysis are seen and in dark grey the samples without lysis.
The respective hosts are: DSM613 for the T7 phage, DSM 613 and ECOR16 for the T3 phage, and Yersinia enterocolitica DSM 23248 for the phiYeO3-12 phage.
Successful co-expression of native T7 and T7p52 (Loop-T3p48) and T7p52 (Loop-phiYe_p51), suggests the system's potential to generate phages with a mixture of tail-fibers which are also modified in the loop region as well as a mixture of tail-fiber for a complete tip exchange, that can infect two different bacterial species.
(B) Titer Assessment:
Titers of the cell-free expressed phages were evaluated on DSM613. The incorporation of modified tail fibers resulted in an enhanced plating efficacy relative to the native T7 phage.

FIG. 9: Multi-specific phage to extend the host-range.
(A) Core phage: Escherichia virus T3 (schematic representation)
(B) Multi-specific phage: Escherichia virus T3 with different tail-fiber

FIG. 10: Modifying host range and specificity through tail-fiber co-expression.
(A) Extension of native T3 Phage Host Range with T7p52 Tail-Fiber co-expression:
Using the T3 phage as a base, co-expression with the T7p52 tail-fiber from the T7 phage was performed. A spot assay determined the phage concentration across two hosts: one specific to both T3 and T7 phages, and another specific only to T7. This demonstrated an expanded host range for the resulting phage, showcasing a novel phenotype.
(B) Influence of Tail-Fiber concentration on T7 phage specificity:
The native T7 phage was employed as a chassis, with varying concentrations of a plasmid encoding the T3p48 while co-expressed. A spot assay assessed the phage concentration on three different hosts. While both T7 and T3 phages can infect all three hosts, they do so at varying efficiencies. The inclusion of different concentrations of the T3p48-encoding plasmid resulted in distinctive phage plating efficiencies. Consequently, the effective concentration on different hosts varies based on the plasmid concentration.

FIG. 11: Table 1

### EXAMPLES

The process demonstrated below provides a simultaneous all in vitro method for the production of multi-peptide structure in a cell-free system.

For this purpose, several coding strands of genetic information (DNA/RNA) were simultaneously added to a cell-free reaction and incubated. This genetic information contains the appropriate structural information, such as the structural information of the asymmetric building blocks from which, for example, a capsid is formed. These are expressed simultaneously in this system, and the specificity of the structure of the proteins leads to a subsequent self-assembly. By varying the concentration of the added coding genetic information, it is also possible to adjust the final concentration of the resulting multi-peptide structure for further use.

The process demonstrated here provides a method for the production of a multi-peptide structure as described herein in a cell-free system.

For this purpose, several coding strands of genetic information (DNA/RNA) are simultaneously added to a cell-free reaction and incubated. This genetic information must contain the appropriate structural information, such as the structural information of the asymmetric building blocks from which, for example, a capsid is formed.

The genetic information is expressed simultaneously in the system. The specificity of the protein structure leads to a subsequent assembly.

By varying the concentration of the added coding genetic information, it is also possible to adjust the final concentration of the resulting multi-peptide structure for further use.

The first step involves the preparation of an E. coli cell lysate. Here, an E. coli based cell extract is used, as these have the highest expression capacity so far (Caschera et al. 2013) and its composition is well known.

The second step involves the provision of the genetic information (DNA/RNA), which must encode specific structural elements. This genetic information must be isolated or physically available e.g. in the form of PCR product, native DNA/RNA, chemically synthesized, as a plasmid or as a yeast artificial chromosome (list not exhaustive).

As an exemplifying embodiment, a genome from the species of teseptimaviruses with a T=7 symmetry is used (DSM 4623) and a similar genome from the same species (DSM No 4621), which are produced simultaneously in a single cell-free reaction. Teseptimavirus is a synonym for T7 phage group or T7-like virus.

The first step for bacteriophage assembly involved the preparation of an E. coli Rosetta™ (DE3) cell lysate.

For this, a cell culture after reaching a certain optical density was harvested and washed. Subsequently, the bacteria were lysed by adding lysozyme and further treated under ultrasound. The latter step improves lysis. The cell membrane, DNA and small metabolites were removed. The remaining solution contained the cell-free extract, which is capable of performing the expression. A buffer containing small metabolites supplementing expression was added.

The genomes of the two teseptima viruses were extracted with the aid of a genome extraction kit (Zymo Research).

These two prepared DNA strands were added to the cell-free system. In this system, the expression of the genes of one teseptimavirus and of the other took place simultaneously. After incorporating the DNA into the capsid, the expressed proteins assembled into fully functional teseptimaviruses, i.e. functional particles.

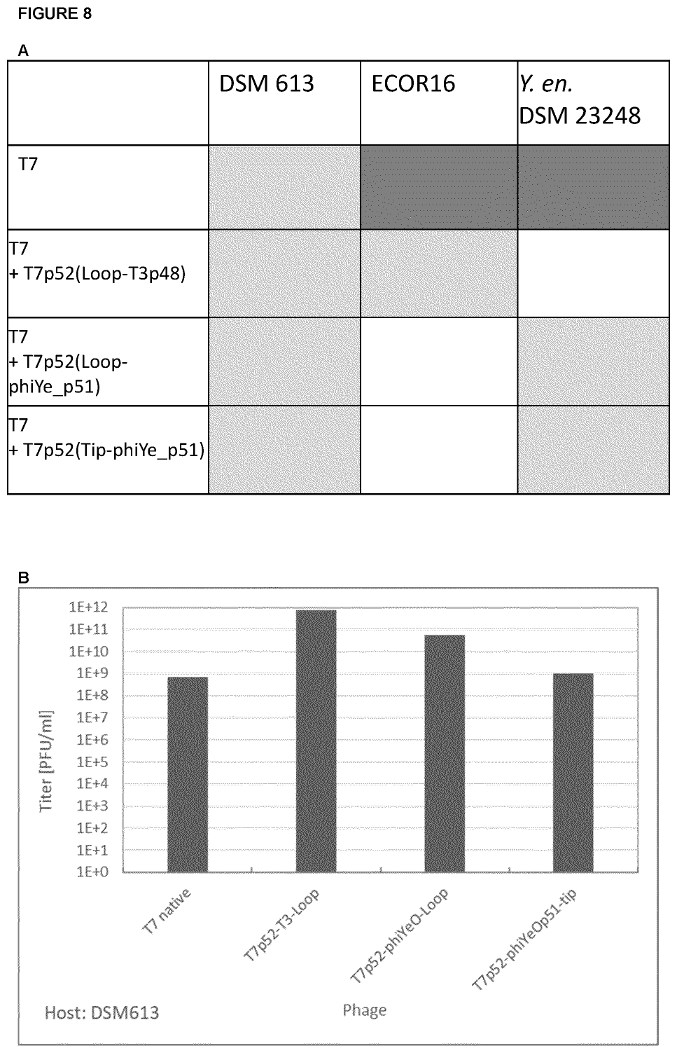

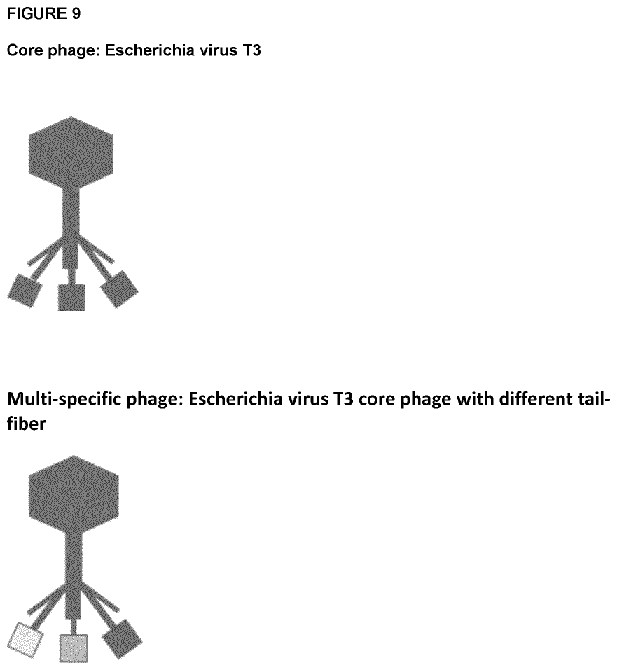

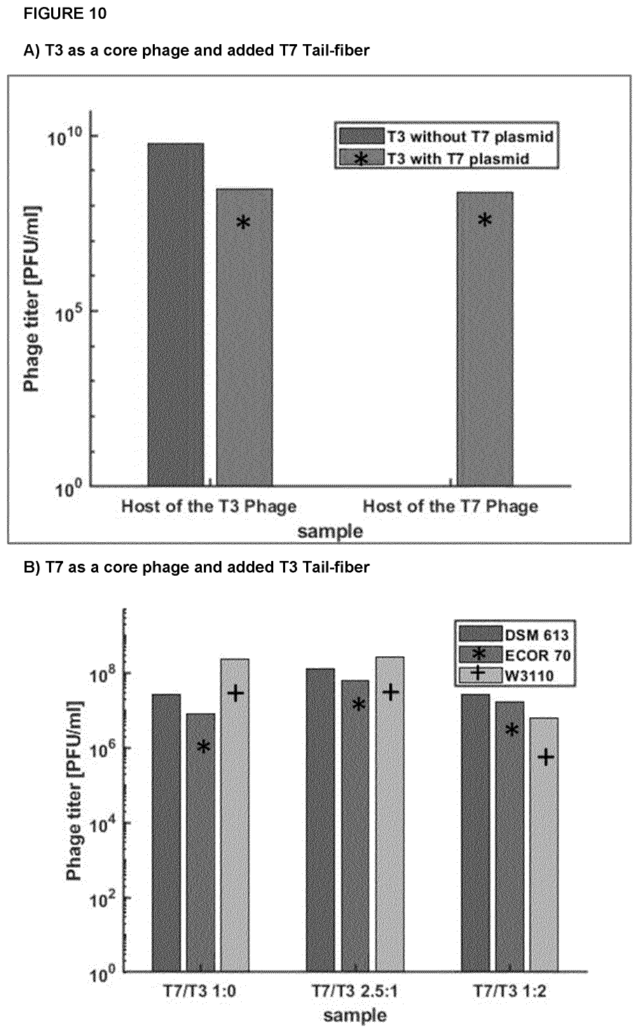

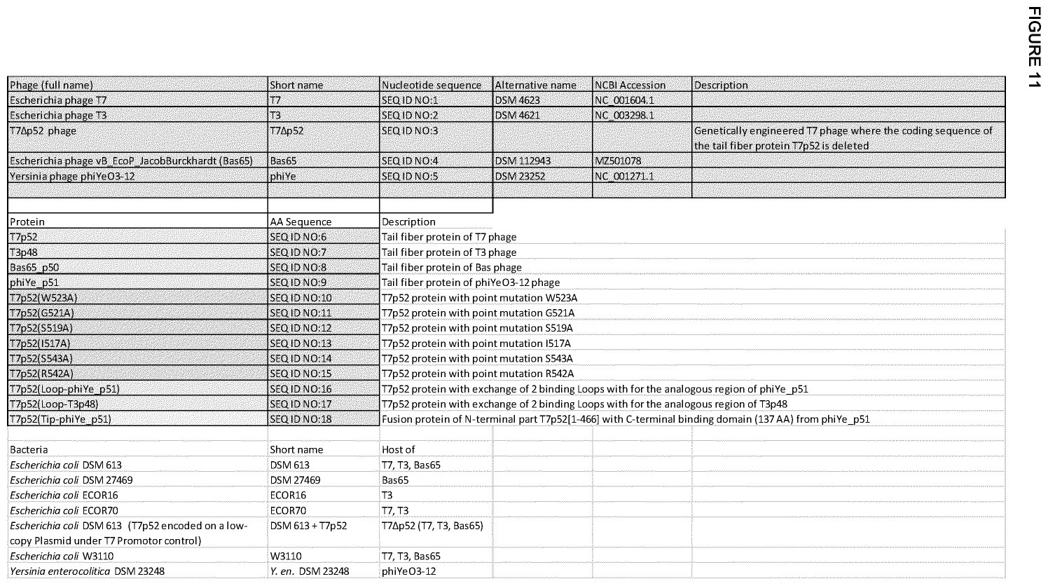

The presence of the functional particles was detected by a spotting assay on the respective hosts and the concentration determined. The expression of the proteins can also be optionally detected by mass spectrometry and the identity of the genetic information by sequencing (c.f. FIG. 3).

FIG. 3 shows the concentration (plaque forming units/ml (PFU/ml)) of different simultaneously expressed particles in cell extract plated on different hosts.

Blue represents plating on host DSM 613 which is the host for DSM 4623, red represents plating on host ECOR16 which is the host for DSM 4621, and yellow represents plating on host DSM 5695 which is the host for DSM 13767.

In sample one, the genome of DSM 4623 and DSM 4621 were simultaneously added to the cell extract and tested against the respective hosts, in the second sample only the genome of DSM 4623 was added to the cell extract and tested against the respective host, and in sample 3 the genome of DSM 4623 and DSM 13767 were added to the cell extract and tested against the respective host. In the last sample the genome of DSM 4623 and a plasmid encoding the gene Gp17 of DSM 4621 were added to the cell extract and tested against the respective hosts.

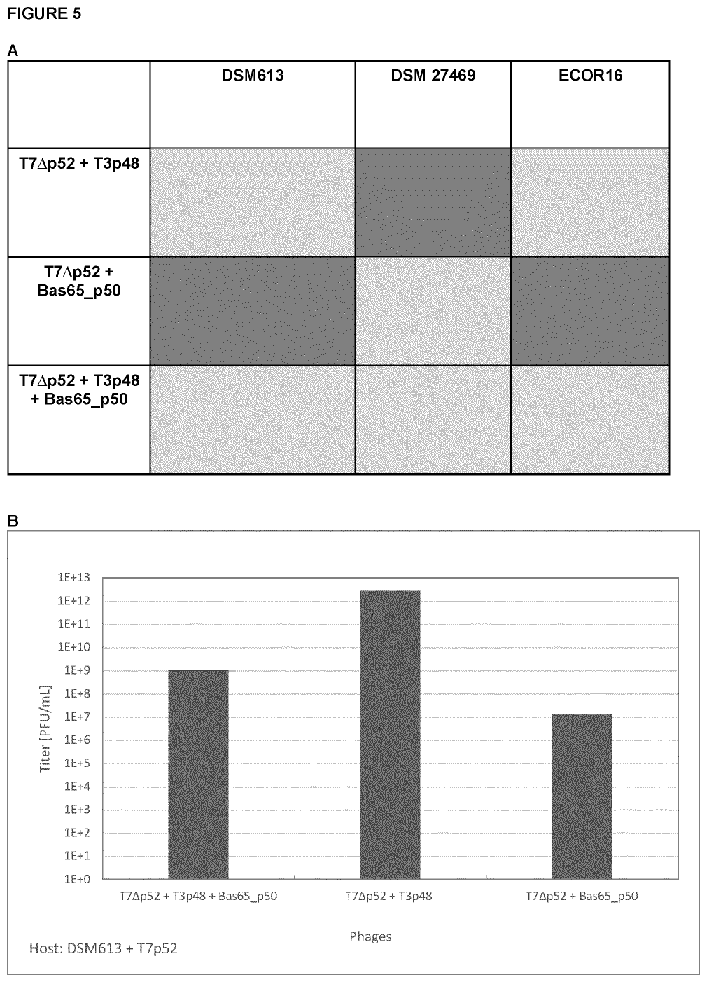

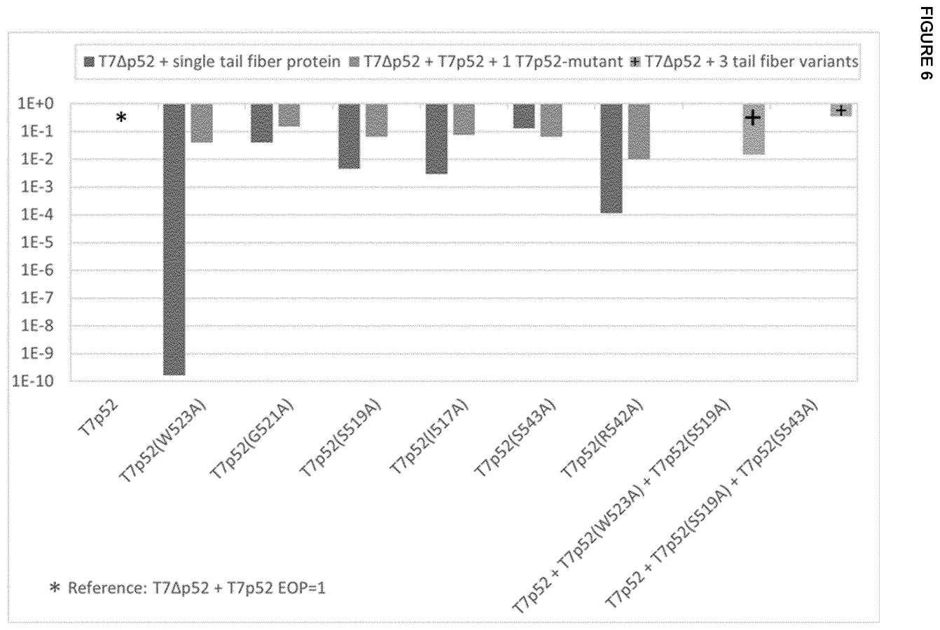

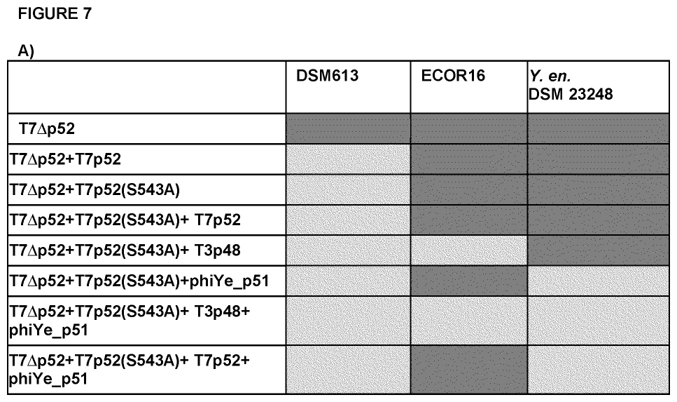

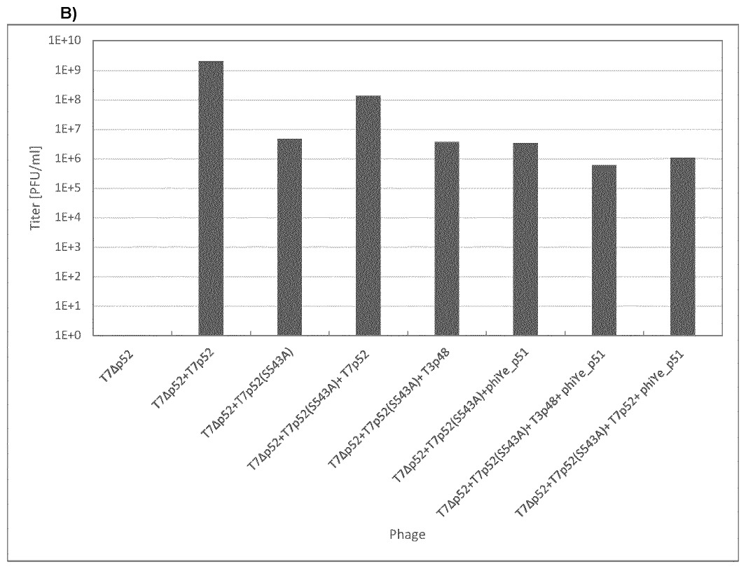

When the genome of DSM 4621 and DSM 4623 were present in the cell extract at the same time, plaques could be detected both when plating with the host for DSM 4623 and the host for DSM 4621 (FIG. 2).

A similar result was obtained when only part of the genome of DSM 4621 was added (plasmid encoding Gp17) and at the same time the whole genome of DSM 4621.

The genome of an emesvirus with a triangulation number of T=3 and the genome of DSM 4623 were also added together in the cell extract. Here, it was also demonstrated that more than 10⁶ PFU/ml particles were present against both the host of DSM 4623 and the host of DSM 13767.

This demonstrated the simultaneous production of particles, i.e. multi-peptide structures for different hosts for gene transfer with different as well as the same triangulation numbers T in a cell-free system.

FIGS. 4-8 show phages co-expressed with tail fibers from other phages, combinations of different natural tail fibers on a phage, coexpression of tail fiber proteins with point mutations as well as phages comprising loop modifications in the tip an/or complete tip exchange. Detailed description is given by the corresponding figure legends.

The following general notes apply.

EOP: Efficiency of plating→Describes the ratio of infection (i.e. the difference in apparent titer) of 2 (or more) phages on the same host OR the infection of one phage on different hosts; The titer is measured for both phages/hosts and the ratio is calculated, choosing one phage/host as reference (divisor)

Experimental setup: The cell-free expression system is used for in vitro expression and assembly of phages by supplying a phage genome as DNA template. In addition plasmids, encoding for additional tail fiber proteins can be added. This co-expression leads to the assembly of phages with more than one tail fiber protein (i.e. a heterologous tail fiber with altered functionality). These phages differ from the natural phage in their ability to infect different hosts, either by expanding the host range (being able to infect new hosts) or by changing the ratio of infection on different hosts (EOP).

Some experiments are performed using T7Δp52—a genetically engineered T7 phage where the entire coding sequence of the tail fiber protein T7p52 is deleted. This genome only assembles into functional (i.e. infectious) phages if a tail fiber protein is co-expressed. After the first infection round, a tail-fiber-less phage is produced, which cannot infect further bacteria. The T7Δp52 is propagated on a plasmid carrying host, which expresses the T7 tail fiber protein T7p52 from a plasmid. This host is denominated: DSM613+T7p52. This host can also be used for quantitative titering of T7Δp52 phages.

Table 1 lists and characterizes the used proteins by sequence as well as the used bacterial strains and phages.

### Multi-Specific Phage to Extend the Host-Range

Goal: Extension of the bacterial host-range of phages by utilizing the modularity of the self-assembly process of phages and the open nature of the cell-free system. By co-expression of several tail-fiber genes, together with a native phage genome in a cell-free reaction a multi-specific phage is produced (cf. FIG. 9).

Experimental design: As a proof of principle T3 phages as core phage are co-expressed with a T7 tail-fiber gene encoded on a plasmid. The concentration of the phages is measured by spot assay on a host bacteria susceptible for T3 phage, as well as on a host susceptible only to the T7 phage.

Further we also used the T7 phage as a core phage with the T3 tail-fiber co-expressed and measured the concentration of these on three different E. coli strains, with different ratios of plasmid to genome, to see alteration in the ratios of effective phage concentration. Here, effective phage concentration refers to the titer of a phage measured on a particular strain of bacteria. This can vary with the properties of the bacteria, like the number of surface attachment sites for the phages.

Results: Phage concentration of a core phage with co-expressed tail-fibers.

FIG. 10 a) shows the titers of the T3 phage as core phage with co-expressed T7 tail-fibers encoded on a plasmid on two different bacteria. A host bacteria of the T3 phage and a host-bacteria of the T7 phage, which is not susceptible to the T3 phage is used to determine the effective phage concentration. In case of the host bacterium of the T3 phage with and without the plasmid plaques are detected. In case of the host bacterium of the T7 phage only in the sample containing the plasmid for co-expression of T7 tail-fibers plaques were detected.

FIG. 10 b) shows the titers of the T7 phage as a core phage with the T3 tail-fiber encoded on a plasmid measured on three different bacteria. The ratio of the effective concentration of the phage on the different host bacteria is altered with the increase of the addition of plasmid. The native 17 phage shows a higher effective concentration on the host bacterium W3110 compared to DSM 613, but with the addition of high concentrations of plasmid encoding the T3 tail-fiber, a higher effective concentration on DSM613 is observed compared to W3110.

Discussion: The results show an extension of the host-range as well as an alteration of the effective phage concentration. When a host-bacterium is used, which is orthogonal to the core phage the host-range can be expanded, as shown with the T3 phage as a core phage and the co-expressed T7 tail-fiber. This also means that in this cell-free one-pot reaction the tail-fiber of both phages are present. While using the inverse system with T7 as a core phage and the T3 tail-fiber also the effective phage concentration on a given set of bacteria can be altered, showing the impact of the additional tail-fiber on the specificity of the phages.

1. A multi-peptide structure comprising at least one heterogenous functional site wherein the at least one heterogenous functional site is composed of at least two homologous peptides, which differ by at least one amino acid.

2. The multi-peptide structure according to claim 1, wherein the at least two homologous peptides are derived from different bacteriophages and/or are engineered in a different way.

3. The multi-peptide structure according to claim 1, comprising a bacteriophage derived nucleic acid, in particular bacteriophage genome, encoding at least one homologous peptide less than comprised by the multi-peptide structure.

4. The multi-peptide structure of claim 1, wherein the functional site is selected from head (capsid), tail, spike, sheath, tube, baseplate or fiber component of a bacteriophage, preferably a capsid or fiber component and even more preferably a fiber tail component.

5. The multi-peptide structure of claim 1, wherein the at least two homologous peptides are selected from the group comprising receptor binding proteins, tail fiber proteins or tail fiber loops, and/or capsid proteins.

6. The multi-peptide structure of claim 1,
wherein the multi-peptide structure comprises at least two homologous bacteriophage tail fiber proteins and/or at least two homologous bacteriophage capsid proteins, preferably derived from different bacteriophages.

7. Method, in particular in vitro method, for providing multi-peptide structures, in particular synthetic bacteriophages, comprising the steps of
(a) providing an expression system, in particular a cell-free expression system,
(b) adding nucleic acids encoding homologous bacteriophage proteins, preferably derived from different bacteriophages, in particular homologous bacteriophage tail fiber proteins and/or homologous bacteriophage capsid proteins, to the expression system,
(c) expressing the nucleic acids encoding the homologous proteins, in particular homologous phage tail fiber proteins and/or homologous phage capsid proteins,
(d) assembling of the expressed homologous proteins, in particular homologous phage tail fiber proteins and/or homologous phage capsid proteins, to provide the assembled multi-peptide structures, in particular the synthetic bacteriophages, and optional
(e) isolating of the assembled multi-peptide structures, in particular the synthetic bacteriophages.

8. The method of claim 7,
wherein the expression system is a cell-free expression system, wherein the cell-free expression system is preferably host independent and preferably selected from cell lysates or artificial expression systems.

9. The method of claim 7,
comprising the addition of bacteriophage specific host factors.

10. The method of claim 7,
wherein at least one of the protein encoding nucleic acids is modified, in particular modified by non-natural mutations such as deletion, insertion and/or substitution.

11. Composition comprising the multi-peptide structure, in particular synthetic bacteriophage, according to claim 1.

12. Composition comprising two or more types of multi-peptide structure, in particular synthetic bacteriophage, according to claim 1, wherein the two or more types of the multi-peptide structure, in particular synthetic bacteriophage, have homologous tail fiber and/or capsid proteins or have different mutations in the tail fiber and/or capsid proteins.

13. An universal anti-microbial agent comprising the multi-peptide structure of claim 1.

14. Multi-peptide structure, in particular a synthetic bacteriophage, according to claim 1 for use in medicine, chemistry, biotechnology, agriculture and/or food industry.

15. Use of a multi-peptide structure, in particular a synthetic bacteriophage, according to claim 1 for avoiding bacterial growth.


SEQUENCE LISTING (18 sequences)

SEQ ID NO: 1 - DNA, 32767 bp, unidentified
  source 1..32767: mol_type = genomic DNA; note = genome from the species of teseptimaviruses (DSM 4623); Escherichia phage T7
tcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatgg

SEQ ID NO: 2 - DNA, 32767 bp, unidentified
  source 1..32767: mol_type = genomic DNA; note = genome from the species of teseptimaviruses (DSM 4621); Escherichia phage T3
tctcatagttcaagaacccaaagtaccccccccatagcccctcttaaagccacccaaagctggccctacccctagggtttaaccttcggttaaccttgggtctttcctcaagtggcctataggatagcctaaagtgatgcctaaagtcaatacctaaagtgatggctgcctattggagacttaaagagtgtcctagtgtaccagtatgatagtacatctctatgtgtccctatctgttagccccttaaagtaaaccctaaagccaccacctaaagagcgacgctatcttaaagacaaccagaccgatagacccactgttaacctgtagtgctgccctgtagttgtctataatatccaacgttgtctatttaccctcactaaagggaataaggtggatacttaaagagggaataaagatttaataaaagattagaattaagtgttgactttaagttacctttaaggctattattcatctcgtcaacgggacaacggccttaaagccactccctgagataccggagtcaaccggataagtagacagcctgataagtcatacgaaaaacaggcattgacaacgcaaggtaacaagtagtaagatgtaccacataacgttaaccaccgcttcggtgtcttctaggtgacttaagcgcaccacggcacataaagttaaacaaagtggttgacaacatgaagtaagcacggtacgatgtaccatatgaaacgacagtgaggcgggacactgaaagccgctagggtctaacgaaaccagaccgacacgctctttaacaatctggataatagctcttgagtgtaccatataagcgaataactcaaggtcgcactgaaagcgtggcctttatgatattcacttaacgaactaacatgaggtaacaccaaatgattttcactaaagagcctgcgaacgtcttctatgtactggtttccgctttccgttctaacctctgcgatgaggtgaatatgagcagacaccgccacatggtaagcactttacgtgccgcaccgggtctttatggctccgttgagtcaaccgatttgaccgggtgctatcgtgaggcaatctcaagcgcaccaactgaggaaaaaactgttcgtgtacgctgcaaggacaaagcgcaggcactcaatgttgcacgcctagcttgtaatgagtgggagcaagattgcgtactggtatacaaatcacagactcacacggctggtctggtgtacgctaaaggtatcgacgggtataaggctgaacgtctgccgggtagtttccaagaggttcctaaaggcgcaccgctgcaaggctgcttcactattgatgagttcggtcgccgctggcaagtacaataagtgttaaactcaaggtcatgcacgatgcgtggcctttatgattaacattctctccatatgaggtattgaaatgctcacagcactaattgcactggtctatctgtgcatcgctatgtactttatgcgagacttcaagcgcggtctggtagtccataaggcaacactgagttactttaagtggggattcatcccacggtttacggttcgtgaacgcaatggacgctttaaggccaataaggtaggagtattctacattgctacgcactaactcaaagcacgtaaagaccgcactgtatgcgatggcgtatggtgcatcgaaacgcaaggtcaaacgcatcttaacgagacaccgcaagatgaccgcacgacaggctgcaagcgctgtcaaatgggctgaatttactctttactcttacagataacaggacactgaacgatgactttcaccgaagcacaagccgcactcgaagctatccgcaacctaccgattgagcgcctgaacgaacgccagactatgctggtggctcttgcggctgccatcgtgaacgctgagacccaagatgggcaattgacgcaggcagcaggaaacacggcgctagaacgcatggactactggcagaccctagcggcgcgcatgagggatgttggtttcatcaagctaggtaatggtcactttagcgcagcgtatcagcacgagatgttgccgggtaaggtcatcaaggtaggctttaagaaagaggattcaggcgcagcctatgcagcattctgccgtatgaatcaagggcgcgaaggtatccctacggtgtacgacattcagcgtcatgcaggctgttacacggtggttcttgactatctgaacccgcttaacgattggtgctatggtgagagtgacaaggtgcgtcaccacttcaaagccgcacgggaaatcattgaggaggattgctttgatgctgccgaagagtacccaataagtgacgggttcattgagacctgcaaggatatccgtaagttcttcttagggatcgcatcgttcgattgtcacagcggtaacatgatgaaagaccaaatgggccgcttgattatcacagacccggtatcgttcagcgcagacgacaagttaaagccgggtgagtttcactgtgacccggatgaactaatagccgagattgaggcgctaagggcacaggaagccattgagcgctgtgtcaatcgtaaggcacgccgtgaccctaaaggagcctttcaggtagcgcgtaaagcacgcgtcaagtggcgcagaaaggccgctaagcgtgagaagtgcaacgctaaggtactcgctaggatgcgcatggacgccaatcagaatcgcagggatgagcctagggcgcgtatggtgtggggcgataagcactggcgcaacgcttggatgcaccacaacaacctaaactttgcagaactggagcgccgcgctgctgcggtaatgatgctgcatgatgaggtgcgcattcagtggggcaaaccgttacacatagacgcataccttgacaagcgtctacaaggctgatagagtcttttcttacaggtcatcatgaggtggcctgaataggaacgatttattcacaatgaggtaagcaatgaacatcatcgaaaacatcgaaaagaatgacttctcagaaatcgaactggctgctatcccgttcaacacactggctgaccactacggaagcgccttggctaaagagcagttggctttagaacatgagtcttatgagctaggcgagcgccgcttcctcaagatgcttgagcgtcaagcgaaagctggtgagattgcagacaacgcagccgctaagccgttactcgctacgcttctccctaagttaaccacacgtatcgtcgagtggctcgaagagtacgcatcgaagaaaggccgcaagcctagcgcatacgcaccgctccagttactcaagccggaggcctccgcgtttatcaccctgaaagttatccttgcgtcactaaccagtacgaacatgacaaccattcaggccgctgctggtatgctggggaaagccattgaggacgaggcacgatttgggcgcatccgtgacctagaagcgaagcacttcaagaagcacgttgaggaacagcttaacaagcgccacgggcaagtctacaagaaagcatttatgcaggtggtcgaggccgatatgattggtcgaggtctgcttggtggcgaggcgtggtctagctgggataaagaaaccacgatgcacgtagggattcgcctgattgaaatgctgattgaatccacgggtctggtggaattacagcgccacaacgcaggtaacgcaggctctgaccatgaggcactgcaactggcccaagagtacgtggacgtattagcgaagcgtgcaggcgctctggcgggtatctctccgatgttccagccgtgtgtcgtaccgccgaaaccttgggtagcaatcacagggggcggctattgggctaacggtcgcagacctttggcactcgttcgcactcactctaagaagggcttgatgcgctacgaagacgtttacatgccagaagtctacaaggctgtgaacctcgcgcaaaacaccgcatggaaaatcaacaagaaagttcttgctgttgtcaatgagattgttaactggaagaattgcccggtagcagacattccatcgctggagcgccaagagttaccgcctaagcctgacgacattgacaccaacgaggcagcgctcaaggagtggaagaaagccgctgctggtatctatcgcttggacaaggcacgagtgtctcgccgtatcagcttagagttcatgctggagcaggccaacaagttcgcaagtaagaaagcaatctggttcccttacaacatggactggcgcggtcgtgtgtacgctgtgccgatgttcaacccgcaaggcaacgacatgacgaaaggtctgctgacccttgctaaaggcaagccaatcggtgaggaaggtttctactggctgaaaatccacggtgcgaactgtgcgggtgttgataaggttccattcccggagcgcatcgcgttcattgagaagcacgtagacgacattctggcttgcgctaaagacccaatcaataacacttggtgggctgagcaggattcaccgttctgtttcctcgcgttttgcttcgagtatgcaggcgttacgcaccacggtctgagctacaattgctctctgccgctggcgttcgacgggtcttgctctggtatccagcacttctccgcgatgctccgcgatgaggtaggcggtcgtgcggttaacctgctgccaagcgaaaccgtgcaggacatttacggcatcgttgcacagaaagtaaacgagattctcaaacaggatgcaatcaacggcacgcctaacgagatgattaccgtgaccgacaaggacaccggggaaatctcagagaagctcaaacttggaacctcaacgctggcgcaacagtggctggcatatggtgtaacccgtagcgtaactaaacgttcggtcatgacgctggcttacggttccaaggagttcggctttcgtcaacaggtattggatgacaccattcagcctgcaattgacagcggtaagggcttgatgttcacccaaccgaaccaagcggctggctatatggctaagctgatttgggatgcggtaagcgtgaccgtagttgcagcggttgaggcgatgaactggctcaaatctgccgctaagctgctggctgctgaggtcaaggacaagaagaccaaggagattctgcgccaccgttgcgcggttcactggactacgccggacggcttcccggtctggcaggaataccgcaagccactccagaagcgtctcgatatgattttcttagggcaattccgtctgcaaccgacgattaataccctcaaggattcaggcattgacgcacacaagcaggagtctggcatcgctcctaactttgttcactcacaggacggtagccacctccgcatgacagtcgtttatgctcacgagaagtatggcattgagtcctttgcgctcatccatgacagctttgggactatcccggcagacgctggtaagctctttaaggctgtgcgtgaaacgatggttatcacctatgagaacaacgatgtgctggcagacttctactctcagtttgccgaccagctacacgagacccaactggacaagatgcctccgcttccgaagaaaggaaacctgaacctgcaagacattctcaagtctgactttgcctttgcataacaagcacttagcattaaccctcactaacgggagactacttaaggtctcccactttaagacactttaggtactaagagattaaatttatgattaacattaagactttctttaagaacatctttaagttaaaccgtttgacttccattaagttctacgcttggatgcccggtagtgatgacctccgcaagactgaatttaagttaggcttagggccgtgcggtaaggtggtcacgaagttggaatgttactcaacgtcctccgggatggtcatcttccagaccactgaggacaacgaaatgaaatccttctactacccgaaaggttcaacgtgtggtcgtatcgaacggacatacagttaaccctcactaacgggagagttaaacttaaggtcatcaccaacggtggcctttgtgattaactttcaatacacatcaacatgaggtaagatactatgcgtaccaactttgagaaatttaccaagcgcgattcagtggtcaacgagcagggcgaacagtggcaagagcgccgtgaccgcatgaagaaacgccacaagcaacagcgcggtaactcacagaaacgggagtggaactgatgatgatgggacgtatttatagcggcaacctgaacgattacaaagatgcggtagcgcgtctacaggaagaccatgatgtgaccgtgaagatggagtcattcagctacgaaaacccagcgaagatgtgcaggtcatgcggtgaggttctcagtgtgttcacacgctccgggcatctggtggcatccagaaccttcgagcatagcgacagcgatgtacaaatcaacgcgcagactgcatggctccgtaaggttcacagcgaattgaaacactggaagtaataaccctcactaacaggagaatccttaaggtcactttaacgagtggccttcgtgattcgaactgattaactttcaaacaatgaggaacaaccgtatgaacatcttcaataccaacccattcaaagctgtgtcattcgtagagtctgccgttaagaaagcgctggagacctccggctatcttatagcggattgtaaatatgatggcgtgcgcggtaacattgtggttgataacgtggctgaggctgcgtggctgtcccgtgtgtccaagtttatcccggcgctggaacacctgaacggtttcgataagcgctggcaacaactgctgaacgatgaccgatgcatcttcccggatggcttcatgctggatggcgaactgatggtcaaaggtgtagacttcaacaccgggtctggcctgctgcgtaccaagtgggtgaagcgtgacaacatggggttccaccttacgaacgtcccaactaagctgaccccgaaaggccgcgaagttatcgacggtaagtttgagttccaccttgacccgaagcgcctgagcgtgcgactctacgctgtcatgccgattcacattgcggagtctggcgaggactacgatgtacagaacctgctgatgccgtatcatgtggaagccatgcgctctcttctggttgaatacttcccggaaatcgagtggcttatcgctgagacctacgaggtctacgatatggattcgctgactgaactgtacgaagagaagcgtgccgaaggtcacgagggtctcattgtgaaagacccgcaaggcatctacaagcgaggcaagaagtctggctggtggaagctcaagcctgagtgtgaagctgacggtatcattcagggtgtcaattggggaaccgaagggttagccaacgagggtaaagtgataggctttagtgtgcttcttgagactggtcgtttagtagacgccaacaacatctctcgcgcattgatggacgagttcacatccaacgttaaagcccacggtgaagacttctacaatggttgggcctgtcaggtcaactacatggaagcgaccccggacggctccctgcgacaccctagcttcgagaagttccgaggaactgaggacaaccctcaagagaaaatgtaaccaactcactggctcaccttcacgggtgggcctttcttcgttccgggcattaaccctcactaacaggagacacacaccatgtggcttatcctattcgctatcgtcgcaacgctgggattaatggttgccgacgacaacatttggcctgattgttaaggagacaacatcatgcgtttacacttcaacacatcaaatggtatcttttcggttcgccgtgaagaccgctctacagcagtggcctctgagcgcaacgccaagctgccgctgattggttcggtcgtcccattgtcaccacgtgttcacctactgatcactcgtggagagttcattaaggcgatgaacaaagagcgcccacatctggaagccgtggtcacttactggcctcgcatccgtctgttcgttaagttgattaaggaggtgctgtaatgttacaacatcattggaacaaaccagacttagaggctcgcttcccggttaactctgttgtgcgatactccggcggtggcctcaagcgcatcttagggatgagcggagtggtcactggtcactcacacacgggtctagttaaggttcgcttcggtactcaatacgctgaggttcttccgaacaacctgattccgctgcctaaagctgacgttaagactccgaacgtggaagcaccgaagacagaagtcaagtctgacgtaacccaccctaaccactacatgctgttcgacaacgtggaagccattgaggttatcgcccggtcaatgacagtcgaggcgttccgtgggtactgcttagggaacatcctcaagtatcgcctgcgtgctggcaagaagtccgaactggcaacgatggataaagacctcaagaaagctgcattctatcaggaactctttgacaaacaccgggggctgtgttatgacgcttcgtgaatgctgcgattggtgtgccgctaagtggaaccaagctattgaggatggtgacaaggcggctgctgacgcttaccaacaactgtacgcgctgtgggagcggcgctttaaggaggcttaacgtggagcaacgtgaaattaaataccttctgacgatggaaggtaacaccgagtcctttgaggttccggtattcgcccggtctctcgaagaggcaactctacaggctgaacaatatgaggacgctgggttcgtagtgactcgcatccgtccagaagtgaaagcctaattaccctcactaaagggaacaacccaacctatcaaccaaaggagaaacaatatggctggtttcaagaagaaagtttacacctccggtctcggtactgctgaaccttatgcttacctgagcaagccggactatggtaacgaagagcgtggcttcggcaaccctcgtggtgtctacaaggtagacctgactctttccaacaaagacccgcgctgtcaggcaatggtcgatgaaatcgtgaagactcacgaagaggcttatgctgctgccgttgaagagttcgaggctaatccgccacaggtacagcgcggtaagaaaccacttacaccttacgagggcgacatgccgttcttcgataatggtgacggtacgactaccttcaagttcaaatgctatgcgtctttccaagacaagaagaccaaagagaccaagcacatcaatctggtcgtggttgatagcaaaggtaagaagattcaggaagtgccgattatcggtggcggctccaagctgaaagtgaaatactcactggtgccctacaagtggaacaccgctgtgggagcaagcgtcaagctgcaactggagtccgtgatgctggtcgaactggctaccttcggcggtggcggtgaagatgagtgggccgatgaagtcgaagatggtggttacactgcgagtgaatcccgtcagtcccgcgatgagcaggagtggcaggaagacgagcacgaagaaaccccggatgatgacgaggacttctaatggctggcgcatacgctgcgaggtgtacgcaaggtcgggctttccgctccggcctagaagataaagtctcaaagcagcttgagagtaagggcattaagttcgactatgaactttggcgaatcccttacgtcatccctgagagtgaccacttatacactccagacttcctgctacctaacggtatcttcattgagaccaaagggctgtgggattccgatgaccgcaagaaacaccttttgattcgtgagcaacaccctgaactggacattcgcttggtcttctcaagcagccgctcaaagctctacaagggtagcccaacaagctacggtgagtggtgcgagaagcatggcattctgtttgctgacaagctgataccagtggctggcgtcaaagaacccaagaaggaggttccgttcgataaatttaagactaagaaaggagtaaagaaaaatggctaaagttcaattcaaaccacgagcaaccacggaggcaatctttgtgcattgctctgcaaccaaaccgagccagaacgttggtgttcgtgagattcgtcagtggcataaagagcaaggctggctggacgtagggtatcacttcatcatcaagcgcgatggtactgtggaagcaggccgtgatgaactggctgtaggttcccacgctaagggctacaatcacaactctattggtgtctgccttgtaggtggtatcgacgataaaggtaagtttgacgctaactttacgccagcccaaatgcaatctcttcgctcactgctggtcacactgctggcgaagtacgaagggtctgttctccgtgctcaccacgatgttgcacctaaagcctgcccgtccttcgacttgaagcgctggtgggagaagaacgaactggttacatctgaccgagggtagacctatgaggatgatttgcattcgttgtgacaagcctttgaagagtcgcgtaccatcacgtcagtgtcagtgcaaagacccgatcaaggctgaggaacacaccgagaagtaattaacactcactaaagggagacttaacgtttccctttgttctcgcaactgattaaggaatgtacaatggaacgagaagacgatagtatctttctgtttcatgctccgtgtgaaaactgtgggtcttctgatggtaactctgtgtactctgacgggcatgaatggtgcttcgtgtgtgaacatcgagtacccgcaaacgaagagcgtgaagcaaagttatcaacgagaagacgtacaggaggcagcaagcctatgagctatgacgtatggaatttcggtgacagcaacggtcgatactctgacctgactgcccgtggcatctcgaaggagacgtgccaaaaggctggctactggctggcgaaagtggacaaccgaatgtatcaggttgccgactacagagaccagaacggctccatcgtgtcacagaaggtgcgcgataaggacaagaactttaagaccaccggaagccacaagagtgatgcgttgttcttgaagcacctctggtctggtggtaagaaaattgtggtcactgagggtgaaattgatgcgctcactgtgatggaactccaagactgtaagtaccctgtcgtatcattgggtcacggtgcctctgctgctaagaagacatgcgccgctaactatgaatactttgaccagttcgagcagattatcttgatgttcgacatggatgacgcaggccgtaaggcagtcgaagaggccgcacaggttctcccggctggcaaggttcgtgttgccgtgttaccgtgtaaggacgccaacgagtgtcacatcatgggtgaagacaaagcaatcttggagcaggtctggaacgcaaacccttgggtgcctgacggtgttgtctctgcgttgtcactcaaagaccgcgttaaggaagctatgacctccgaggacgccgtaggtttattattcgatggttgtcaaggtctgaacgatagaaccttgggtgctcgtggtggtgaagtcgttatggtaacttccggttccggtatgggtaagtcaactttcgtccgtcaacaggcgctggcttggggtaaacggatgggtaaacgtgtcggcttagcgatgctggaggaatccgttgaggacaccattcaggatatgatgggcctgaacaataaggttcgtctacgtcagtctgatgaagtcaagaaggctatcgcggaggatggacgtttcgatgaatggtatgatgaactattcggagacgacacgttccacctttatgactcctttgcggaggccgaagctgacagactgttagcgaagctggcctacatgcgaactggactagggtgcgatgttatagtgctcgaccacatctcaatagtcgtatctgcctctgaggaatcagatgagcgtaagatgattgaccgcctcatgacgaagctcaaagggttcgctaagtcaactggcgttgtgctcgtagtgatttgtcacttgaagaacccggagaaggggaaagcgcatgaagaaggacgcgctgtgtctattactgacctacgtggtagcggtgccttgcgtcagttatctgatactatcatcgccttggagcgtaaccagcaaggtgatatgcctaacttggtgttggttcgcctgcttaagtgtcgtttcactggcgacactggaattgctggatacatggaatacaacagagaaaccgggtggcttgaaccgtctagctacactggcggagaaggagaaggagatactggctggactgaacaagacggacagtcagacttctaaaggatgcccgtgtgggcattccgattgcgatggaatcatgtaccactaattaaccctcactaacgggaacaacctcaaaccataggagacacatcatgtttaaattcattaacactttaggtaagctggtagtcaaactgtacttcatcgaagccaagaagctggacaagaaagccaaagctgattcacagcgtgccattgagttggcgaaacagtcccgtgagaaatccgatgcggctgttagtggcgtccataagtcggcagcgattgcagccaaagcgcagtccatgagcaaattctttgagtaataggagcacaacatgagtaaatcaatcaagcacgcaaacaccatccgtctgccggacactgctgaccagttcgctcgtcgggtacacattaatgtgcgtggcgagaaggtcacaatggtctaccgctggaaagaccacaagtctccgaaggcacacactcagcgtatgaccttggacgacaagcaggtaggccgcctgatgggtgccctgactatggctgctgacaatgtggttggggataaccgcgagcgtctggtagagttcggcgctggtatgcaagagattatcgagaagtgataaactcaaggtcgcccaagggtggcctttatgattatcatttagcacgaaaccaaaggagggcattatgctcgtaagtgacattgaggccaacaacctcttagagaaagtcaccaagtttcactgtggcgtcatctatgactaccgtgatggtgagtatcatagttatcgtccgggcgacttcggggcgtatctggatgcgctggaagccgaggtgaagcgaggtgggcttattgttttccataacggtcacaagtatgacgttcctgcattgaccaaactggcaaagctgcaattgaaccgagagttccaccttcctcgtgagaactgtatcgacacccttgtgctgtcacgcttgattcactctaacctaaaggacaccgatatgggtcttctgcgttccggtaaacttccggggaaacgctttgggtctcacgctctggaggcgtggggttatcgcttaggcgagatgaagggtgaatacaaggatgactttaagcgtatgcttgaagagcaaggcgaagaatacgttgacggaatggagtggtggaacttcaatgaagagatgatggactataacgttcaggacgttgtggtcactaaggctctccttgagaagctactctctgacaaacattacttccctcctgagattgactttacggacgtaggttacactacgttctggtcagaatcccttgaggccgttgatgttgaacatcgcgctgcatggctactcgctaagcaagagcgcaatggattcccgtttgacacgaaagcaatcgaagagttgtacgtagagttagctgctagacgctctgagttgctacgtaacttgaccgaaacgtttggctcgtggtatcaacctaaaggtggaacagaaatgttctgccatccacgaacaggtaagccactacctaaataccctcgcattaagatacctaaagttggtggtatctttaagaagcctaagaacaaggcacagcgagaaggccgtgagccttgcgaactggatacccgcgagtacgttgctggcgctccttatactccagtggagcacgtagtgtttaacccttcgtctcgtgaccacatccagaagaaactccaagaggctgggtgggtcccaactaagttcaccgataagggtgctcctgtggtggacgatgaggtactcgaaggagtacgcgtagatgaccctgagaagcaagcggctatcgacctcattaaagagtacttgatgattcagaagcgaatcggacagtctgctgagggagacaaggcatggcttcgttacgttgctgaggacggtaagattcatggttctgttaaccctaatggagcagttacgggtcgtgcgacccatgcgttccccaaccttgcgcaaattccgggtgtacgttctccttatggagagcagtgtcgcgctgcttttggcgctgagcaccatttggatgggataactggtaagccttgggttcaggcaggtatcgacgcatccggccttgagttgcgctgcttggcccacttcatggctcgctttgataacggtgagtatgctcacgagattcttaatggtgacatccacactaagaaccagatggctgctgaattacctacccgtgataacgctaagacgttcatctatgggttcctctatggtgctggtgatgagaagattgggcagattgttggtgctggtaaagagcgcggtaaggaactcaagaagaaattccttgagaacacacctgcgattgcagcactacgtgagtctatacaacagacgcttgtcgaatcctctcagtgggtagctggtgagcagcaagtcaagtggaaacgccgctggattaaaggtctggatggtcgtaaggtacacgttcgtagtcctcacgctgccctgaataccctactgcaatccgctggtgctctcatctgcaaactgtggattatcaagaccgaagagatgctcgtagagaaaggcttgaagcacggctgggatggggactttgcgtacatggcatggatacacgacgaaattcaggtagcttgtcgtaccgaagaaatcgctaagactgtcatagaggtcgcacaagaagcgatgcgttgggttggagagcattggaacttccgttgcttactcgacaccgagggtaagatgggagccaactggaaagaatgtcactaatggaggtattatgggaggcgttgcaacattgcggtttcacaagatgcctaccggatgtatcgtgtgcgtttcccataagagaaaccaagatggatacttccggtatagcatagggtcttctcgtaaaggtgagaagaaacactttatgtttcatcgatgggtgtgggaacaacataaaggtccgataccagatggttacgagatagaccacatatgtcttaatagaggttgttgtaacgtagagcacctacagtgtataccaaagcgagaccacattattaagacaaacaaagagcgtaaacttgtcaagaaataaaggagaaattattatggctatcactaagaaatttaaagtgtcctttgatgttacccttaagctggacagcgagaccgaagaactgatgcgagaaaagattctggacttagcgcatcgtgctggtgctggtgaggagattactcctatggaccgagagttgctcgttcaggccctaacacgcggtccagacggtgctgctgcatttgctgtacgccaaggtatccgtaatgctgtcaaggagatgttcgaagagactagtgataaatcactgttaaaactctctcctgcaactgtacgggaggtgttctgatgtctgactacctgaaagtgctacaagcaatcaaaggttgccctaagactttccagtccaactatgtacggaacaatgcgagcctcgtagcggaggccgcttcccgtggtcacatctcgtgcctgaccacaagtggacgtaacggtggcgcttgggaaattactgcttctggtactcgctttttgaaacgaatgggaggttgtgtctaatgtctcgtgaccttgtgactattccgcgcgatgtgtggaacgatatgcaaggctacatcgactctcttgaacgtgagaacgatagccttaagaatcaactgatggaagctgacgaatacgtggctgagctagaggagaaacttaatggcacttcttgaccttaaacaattctacgagttacgtgaaggctgtgacgacaaaggtatccttgtgatggatggagactggctggtcttccaagcgatgagtgctgctgagtttgatgcctcatgggaggaagagatttggcaccgctgctgcgaccacgctaaggcccgtcagattcttgaggattccattaagtcctacgagacccgcaagaaggcttgggtaggtgctccgattgtccttgcgttcaccgatagtgttaactggcgtaaagaactggttgacccgaactacaaggctaaccgcaaggcaaccaagaaacctgtaggttactttgagttccttgaggctctctttgagcgtgaagagttctattgcatccgtgagcctatgcttgaaggtgatgacgttatgggagttatcgcttccaacccgtctgcattcggtgctcgtaaggctgtaatcatctcgtgcgataaggactttaagaccatccctaactgtgacttcctgtggtgtaccactggtaacatcctgactcagaccaaagagaccgctgattggtggcatctcttccagaccatcaagggtgatatgaccgatggctactccgggattcccggctggggcgatacggcagaaggtttcctgaatgacccgttcattgtggagcctgtagagtccgtgttgaagtctggtaagaacaaggggcagacagtaaccaagtgggtgaagcgtgctcctgatgcgactgagactctgtgggactgcattaaatccattggtgcaaaggctgggatgaccgagcaggaaatcatcaagcaaggccagatggctcgcattcttcgcttcgaggaatacaattacattgacaaggagatctacttatggacgccgcgaagctgattaccgtcgctgtgctggtaggtatgctggcgcgtggtctctggatgttcgctctcatagtccgagacgtgaagcgagactcagagattaagtgataaactcaaggctctctacaattaaccctcactaaagggaagagggagcctttatgattattactttaagacttaaccctaagaggtgaaattatgttacaaccaattaatcatgttttaactcatcccgatgatattcctagtatgcctcgtgcagctaaagagtatctacaggtgcgcttcaatcaggcttatgtgatggagtccggggagtaccgtgcgctccgcgctgctggatactctgagtctttcattgcgggtgtgatgcatggtctgtaccttgcctctcgtacacttgatgagattgaggtgcgtaaagagcaactgagacaggagtaattatatgtgctggaaaccgaaggtgaaagtccctaagatggacactaaccagattcgcgctgccgaaccagcacctttgactgaaccaccgaaatctgtggtatggggaggcgatgacgatgaggacaccagcgtgtcttcctctgaggtgccaaccacaccgagcagcggtaagagcagtctaaaggtaaagctggacgacagcgcagccaagaacaagagtaaatcctccattcgctccaaggcgtttgggtaattgtgacataacataggggacacattatgggattcttcaagaaaatcaagaaagctgttaagaaagtagttaaggaagttggtaaaccgatcgagaaggctggtaaggaagtcggtaaagccgttggtggggcgctgggtgctggtaagcaggagattatccaacaagaggctccggcaccagtagtagctgcaccaccaccagcacaggttgtagatgtacctgaacaggataaggctgagggtgaggatgaggcgcaaaccgagagcgcacgtaagaaggcccgcgctggtggtaagaaggcacttagcgtagctcgcagttccggcggtggaatcaatatctgaggaggactgaatggctgattcaaaacgtacaggattgggcgaagacggtgctaaagctacctatgaccgcctaacaaacgaccgtagagcctatgagactcgtgcggagaactgtgcgcaatacaccattccgtccttgttcccgaaggagtccgataacgaatctaccgactacacgactccgtggcaggctgtaggtgcgcggggtctcaacaatctagcctctaagttaatgcttgcgttattcccgatgcagtcgtggatgaagctgaccattagcgaatatgaggcgaagcagcttgttggagaccctgatggactcgctaaggtggacgaaggtctgtcaatggttgagcgcataatcatgaactatatcgaatccaacagttaccgcgtaacactctttgagtgcctcaagcagttgatcgtggctggtaacgccctgctttacttaccggaaccagaaggtagctacaatccgatgaagctgtaccgattgtcttcttatgttgtccaaagagacgcatacggcaatgtgttacagattgtcactcgtgaccagatagcctttggtgctctcccggaagacgttaggtctgcggtagagaaatctggtggtgagaagaagatggacgaaatggtcgatgtgtacacccatgtgtatctcgatgaagagtccggcgattacctcaagtacgaggaagtagaggacgttgagattgatggttccgatgccacctatccgactgacgcgatgccctacattccggttcgcatggttcgcattgatggcgagtcttacggtcgctcctactgtgaagaatacttaggtgacttaaggtcgcttgagaatctccaagaggctatcgttaagatgagtatgattagcgcgaaggtcattggtctggtgaacccggctggcattacgcagccccgtagattaaccaaagctcagactggtgacttcgttccaggccgtcgagaagatattgacttcctgcaactggagaagcaagctgactttaccgtagcgaaagctgtgagtgaccagatagaagcacgcttatcgtatgcctttatgttgaactctgcggtacagcgcacaggcgaacgtgtgaccgccgaagagattcgatacgttgcgtcagaactggaagatacgcttggtggcgtctactcgattctgtctcaagaattgcaattgcctctggtacgtgtgctcttgaagcaactccaagcaacctcgcagattcctgagctaccgaaagaagccgttgagcctactatcagtacaggtctggaagcaattggtcgtggtcaagacctcgataagctggagcgttgcatctcagcgtgggcggctcttgcccctatgcagggagacccggacattaatcttgctgtcattaagctacgcattgctaacgctataggtattgatacttctggtatcctactgacggatgaacagaagcaagcccttatgatgcaggatgcggcacaaacaggcgtcgagaatgctgcggctgctggtggtgctggtgttggtgctttggctacctcaagtccagaagccatgcaaggtgctgctgccaaggctggcctcaacgccacctaattaccctcactaaagggagacctcatctttgaaatgagcgatgactaaaggttggagtcctttggtttccctttatctttaataacttaggagatttaataatggctgaatctaatgcagacgtttatgcgtccttcggtgtgaacaacgcggtaatgaccggaagcacacctactgaacacgaacagaatatgctgagtctcgacgttgctgcccgtgatggcgatgatgcaatcgtgcttagcgatgaaccgacttcccataacgatgacccctatgcggcaggtgtagacccgttcgctgatggtgaagatgatgagggccgcattcaggttcgtatcagtgaagatggcaatgaagccgggttcgacactgatggcgataactctgaggtggagaccgaaggtgaggacgttgagtttgaaccgctgggtgacaccccagaagaactaagccaagtgactgagcaattaggtcagcacgaagaaggctttcaggcgatggtcgagcaggcagttgagcgtggactgagcgcagactctgtgagtcgaatctacgaagagtatgaagccgatggcatctccgagaaatcctatgctgaactagaagctgctggctatagtcgtgcctttgtggactcatacatctcaggtcaggaagctctggtagaccagtacgtcaatcaggtagttgcctttgctggtggtcaggagcgctttagtgcaatccatactcacctcgaagcgacaaaccctgctgctgctgagtcccttgagtctgccatgatgaaccgagacttggcgaccgtcaaagcgattatcaatctggctggtgagagctacacgaagaaattcggtaagcctgccaaccgtagtgttaccaagcgtgctactccggttaaacccgtagctcgtcagaaagagggctttacgaatcaggctgagatgattaaagctatgagcgacccgcgctatcgtagcgattctgcctaccgccaaatggtagaacagaaggttatcgactctagtttctaattaaccctcactaaagggagagaccatagatgcctacaatggttgaatcacctgagcacagaactttgtggtcactcccataggtgaaacattgagaaccaactcgattcaagtagtaaccaaacttttctttaaattaacataaggagattcaacatggctaacattcaaggcggacagcaaattggtactaatcagggtaagggtcagtccgcagcggacaaattggcgctgttcctgaaagtgttcggcggtgaagtcctgacggctttcgctcgcacctccgtgaccatgcctcgtcacatgctgcgctctattgcttctggtaagtccgcacagttccctgtgattggtcgcaccaaagctgcttacctgaaaccgggtgagaacctcgatgacaaacgtaaagatatcaaacacaccgagaaggtaatccacattgatggcctgctgactgcggatgtgctgatttacgacattgaggacgcgatgaaccactatgacgttcgcgctgagtacaccgcccagttgggtgaatctctggcgatggcggctgacggtgctgtactggcagaactggctggtcttgttaatctgccggacggctctaacgagaacattgagggtctcggtaagccaaccgtactgactctggttaagcctaccactggcagcctgactgacccggttgagttgggtaaagcgattattgctcagttgactatcgctcgtgcatccctgaccaagaactacgttccggctgctgatcgcaccttctacaccactcctgacaactactctgcgattctggctgctctgatgccgaacgcagcaaactatcaggcactgctcgaccctgagcgcggtactatccgtaacgtgatgggcttcgaggtggttgaggttccgcacctgaccgctggtggtgcaggcgatacccgtgaggatgccccggctgaccagaagcacgctttcccggctacttccagcactaccgttaaggttgctctggataacgttgtgggcctgttccagcaccgctctgcggttggtacggtcaaactgaaagacttggctctggagcgtgctcgtcgtgcgaactatcaggctgaccagattatcgctaaatatgcgatgggtcacggcggtctgcgtccagaagctgctggcgctatcgtgctcccaaaggtgtcggagtaattcccgaccccactggagttacgttaagtcaaaagaccatgacgcttgttgagggtgcatcccgtgccctcacaggaacagtccagccttccgatgcgaatcagtcgttaagctggtcgtcttctgcggaagatgtggctgtatgggaagctggaaaggttgtagccaaaggtgtgggaacggcggacattacagctactacctccaatggcctgatcgcttcctgtaaagtgattgttaacgccgcaacgtcttaaaacacgtagtgcccaattagggcatcatgtggcgtgaagcactctcaagccgcgaagtggcacatacgtctgactatggggactaccaagagctacgactatcatgtgggcaaccaccctgaaagctcgttgtgattgggataacaatctactaatatgcaaaccccttgggttccctctttgggagtctgaggggttttttgctttaaccctcactaacaggaggtaacatcatgcgctcttatgagatgaacattgagaccgcagaagagctatcagccgtcaacgacattctggcttccatcggtgagccaccagtatcgacccttgagggtgatgcaaacgctgatgttgcaaatgctcgacgtgtactcaacaagattaaccgacagattcagtcacgaggatggacattcaacatcgaagagggcgtgactcttctaccagatgcattctctggcatgataccgtttagctctgactatctgtcagtaatggctacaagcggtcagacccaatatgtcaaccgtggtggctatctctatgaccgctctgcgaagactgaccgatttccttctggtgttcaggtcaacctgattcgtctgagagagttcgatgagatgcctgagtgcttccgaaactatatcgttaccaaggcttcccgccagtttaacaaccgcttctttggtgcgccggaggtagacggagtgttgcaggaagaggaacaggaagcatggagtgcgtgcttcgagtacgagctagactacggcaactataacatgctggatggtgacgcattcacctctggtctacttaaccgctaatcataaggaggctctatggctctcattagtcaatcaatcaagaacctcaagggtggtatcagtcaacaaccagatattctcaggttcactgaacagggtagcgtacagattaatggctggtcttctgagtcagagggtatccagaagcgcccaccgatgattcaccttaagacccttggcactgctgggtatgtgggtgcgcaaccttacgttcacctcatcaaccgggatgagttcgagcagtatttcgttgtgttcactggtgaagatattaaggtattcgacctcgatggtaaggagtaccaagtccgtggagaccgctcatatgttcgtacagcaaacccacgggaagaccttcggatggtaacggtggctgattatacctttgtgactaaccgcaaggtggttgtgcagagtaacgaccaatctgtcaaccttccgggatttaaagaccaaggcgatgcgctaattaacgttcgaggtggacaatatgggcgtagactctcaatcgagttcaacggggctgagcgtgctgctgtgcaactaccagacggttcacaaccagcacacgtcaatgaggttgacggtcaggccatcgctgagaagctggcggctcagttgcggaataacctagggaaccccaacaatgaccaagacccaaataagtggcggttcaatgttggccccggattcatccatatccttgcgccaaataacgataacgtttggggactacagactaaggatggttacgcagaccagctaatcaaccctgtaacccattacactcagtcgttccagaagctgcctattaacgcgccagatgggtacattgtgaagattgtaggtgacacctccaagactgccgaccagtattacgttaggttcgacctcaaccgtaaggtgtgggtagaaacaatcggctggaacacaaggactcacctgcactaccacacgatgccttgggcgctggtacgcgcttctgacgggaactttgacttcaagtaccttgagtggggtgctcgtactgttggcgatgatacaactaacccatacccttcctttaccggacagacaatcaacgatattttcttctttcgtaaccgtttaggattccttagcggggagaacatcatcttgtctcgcacctcgaaatacttcaacttcttcccggcctccgtgtcaaactactccgacgatgacccaatcgacgttgcggttagccatgaccgggtgtcaaccttgaagtatgccgttccgttctccgaagagttgctcctgtggtctgaccaagcgcagttcgttctgacagcctctgatatcctttcgagtcgctccgtagggttgaaccttacgactcagttcgacgtgcaggacagggcacgcccacatggtgtagggcggaatgtatacttttcgtcacctcgcgcttccttcacatccattaaccgatactatgcggtacaggatgtaagctctgtgaagaacgcagaggacatgacggctcacgttccgaactacataccgaatggggtatttagtatctcaggaacaactgctgagaacttcgtggctatccttacgagcggtgcgccgaaccgagtgtatatctacaagttcctctacattgacgaggaaatcaggcagcagtcgtggtctcattgggactttggggacaacgttacggtgttcgcagcacaggtcataaactcaacaatgacagtattgatgagcaatgaacatgctgtgtggatggggcgcttacacttcacgaaggactccatagaccttccgggagaaccttataggttgtatatcgacgctaagagaaagtacaccatccctgctgggacgtacaacgatgatacctaccagacgtcaataagcctctcaacgatttacgggatgaacttcacgaagggtagggtttcggtggtctttcctgatgggaagattgtagagatagaccagccaatcaacgggtggagtagcgacccaatgctgagacttgatgggaaccaagagggacaagtagtctacattgggttcaacatcccattcacgtatacgttctcgaagttcctcatcaagaagaccgctgaggacggctcgacggctactgaggatatcggtcgcttacagcttcggcgtgcatgggtaaactatgaggactctggagcgttcatcatccgtgtgaacaacctgtcccgtgagttcatctacacaatggcgggtgcacgattggggtctgacaacctccgtgttggaggctcaaacattggtacagggcagtaccgcttcccggtggtaggcaatgctcagactaacacagtaaccatcgagtcagatgcgtcaaccccattgaacatcattgggtgcggatgggagggtaactacctgagacgttcttctggtatttaactcaaagtctccctgcggtgaattaaccctcactaaagggagacactaatagatacgaagggggggggggggggttaaagcattatgtatattacaaagtgtttacaaagccacgctgacagctttaagccgtccatagaggacattctggaagctgaggcattgggtgtcgaacctaaagtaattcctgatgagaacactgtggcgatgctaagcgatagcgctgtgctgtccattggtggaaatcagggagaccgagtatggttccttacctctaagtacgtcccgctgtttaccttgaaggagcgcttagagtttcgtagacttatcattgaatatcgtgatatgatgctgagccagtacgagtccatttggaatttcgtttgggtaggcaataagtcacacattcgtttcctaaagaccataggtgcggtattccataatgagtttaccgctgatggtcaatttcaattatttactatcagtaggaggtaactatgtgctggatggcagcgattcctattgctatggcgggtgcccaagctctaagtagccagaacagtgctgacaaggcgcgcgtggcacagacagaagctggacgccgacaggcaattgagatggtaaaagagatgaatatccaaaatgccaacgcctcattggaacaacgggacgcccttgaagctgcatcctctgagttgacttcacgtaacatgcagaaggtacaggctatgggaaccatccgtgcagcgattggtgagggtatgctcgaaggtgaatccatgaagcgcatcaagcgtatcgaagaaggcaactacattcgggaggcaaatagtgtcaccgagaattaccgccgagactacgcgagtatctttgcgcaacagttgggacgcactcagtccacagcaagtcaagtcgatgcaatgtacaagagcgaggccaaaggtaagtctggtctgatgcgtgtactagaccctctgtccattatgggtcaggaagctgcaagtcaatatgcggctggtggatttgacaagaaaggtggcaaccaagcagcacctatcagtgccgccaaaggaactaagaccgggaggtaataatggctagtaaactaaatagtgttttaggcaacatggcgactcccggtatggaacgactcaggggagtccgggggatggactacagggcagcaaccattcaggcagagcaacctagagcgagtcttctggactccattggtcgattcgctaaggctggtgccgatatgtatatcgctaaggaacaacgagcacgcgacctagctgacgaacgctctaacgagattatccgtaagctgacacctgagcaacgtcgagaggctctcaacaatgggacccttctgtatcaggatgacccatacgctatggaagcactgcgagtcaagactggtcgtaacgctgcgtaccttgtggacgacgacgttatgcagaaggtcaaagaaggtgtcttccgtactcgtgaggagatggaacagtatcgccatagtcgtcttcaagagggcgctaaggcatacgctgagcagttcggtattgaccctgaggacgttgattatcagcgtggtttcaatggggacattaccgagcgtaacatctcactgtatggcgcacacgataacttcttgagccagcaagctcagaagggtgccatcatgaacagccgagtggaactcaacggtgtccttcaagaccctgatatgcttcgtcgcccagactctgcggacttctttgagaagtacatcgacaacggtctggttaccggagcaatcccgtctgacgctcaggctacacagcttataagccaagcgctcagtgacgcttctagccgtgctggtggtgctgacttcctgatgcgagtcggtgacaagaaggtaacacttaatggagccactacgacttaccgagagttgatgggtgaagaacagtggaatgctctcatggtcacagcacaacgttctcagtttgagaatgacgctaagctgaacgagcagtaccgcttgaagattaactctgcgctgaaccaagaggaccctcgtactgcgtgggagatgcttcaaggtatcaaggctgaactcgataaggttcaacctgatgagcagatgacaccgcaacgtgagtggctaatctcggcacaggaacaagttcagaatcagatgaacgcatggacgaaggctcaagccaaagctctggatgactcaatgaagtctatgaacaaacttgacgtaatcgacaagcagttccagaagcgaatcaacggtgagtgggtctcaacggacttcaaggatatgccaacaaacgagaacactggtgagttcaaacatagtgatatggttaactacgccaataagaagctcgcggagattgaccgcatggacatcccagatagcgccaaggacatgatgaagttgaagtaccttcaagcggactctaaggacggggcattccgtacagcaatcggaactatggtgactgacgctggtcaagagtggtctgccgctgtgattaacggtaagttgccagaacgaaccccagctctggatgctctacgtagaatccgtaatgccgacccccagttgattgctgcgctatacccagaccaagctgagctattcctgacgatggacatgatggacaagcagggtattgaccctcaggttattcttgacgctgaccgactgactgccaagcgttccaaagagcaacgattcgaggacgataaagcattcgagtctgcattgaatagctctacggcccctgagattgcccgtatgccagcgtcacttcgtgaatctgcacgtaagatttatgactcagttaagtaccgctctgggaacgaaagcatggctatggagcagatgaccaagttccttaaggaatctacctacacgttcactggtgacgatgttgacggtgataccatcggtgtgattcctaagaacatgatgcaagtcaactctgacccgaaatcatgggagcaaggtcgcgatattctggaggaagcacgtaagggaatcattgcgagcaaccagtggataaccaacaagcaactgaccatgtattctcaaggtgactccatttacctcatggacaccactggtcaagtccgcgtccgttatgataaagagttactctcgaaggtctggagtgagaaccagaagaaactcgaagagaaggctcgtgagaaggctctggctgatgtgaacaagcgggcacctatcgttgccgcaacgaaggcccgtgaatctgctgctaaacgagtccgagagaaacgtaaacagactccgaagttcatctatggacgcaaggagtaactaaaggctacataaggaggccctaaatggataagtacgataagaacgtaccaagtgattatgatggtctgttccaaaaggaggctgatgccaacggggtctcttatgaccttttacgtaaagtcgcttggacagagtcacgatttgtacccacagctaaatctaagactggaccattaggcataatgcaattcaccaaggcaaccgctaatgcccttggtctgcgagttacagatggtccagacgacgaccgacttaaccctgagttagctatcaacgctgccgctaagcaccttgcaggtctggtaggtaaattcgacggtgatgaacttaaagctgcccttgcatacaaccaaggcgagggacgcttgggtaatccacaacttgaggcatactctaagggagacttcgcgtccatctctgaggaaggacgtaactatctgcgtaaccttctggatgtcgctaagtcaccgatggctggacagttggaaacttttggaggtatcaccccaaagggtaaaggcattccggctgacgttggactagctggaatcgaccacagtcagaaagtaacacaggaacttcctgagtccacaagttttgacgttaaaggtatcgaacaggaggctccggcgaaaccattcgccaaggacttttgggagacccacggagaaacacttgatgagtacaacagtcgttcaaccttcttcggattcaaaaatgctaccgaagctgaactctccaactcagtcgctgggatggctttccgtgctggtcgcctcgataatggttttgatgtgtttaaagacaccattacgccgactcgctggaactctcacatctggactccagaagagttagagaagattcgaactgaggttaagaaccctgcgtatatcaacgtggtaactggtggttctcctgagaatcttgacgacctcattaagttggctaacgagaactttgagaatgactcacgctctgccgaagctggactgggtgccaaactgagtgctggtattatcggtgctggtgtggacccactcagctatgttcctatggtcggtgtgactggtaagagctttaagttaatcaataaggctcttgtagttggtgccgaaagtgctgcgctgaacgttgcatccgaaggtctacgtacctccgtagctggtggtgacgctgactactcaggggctgctttaggtggctttgtgtttggtgctggtatgtccgcaatcagtgacgctatcgctgctggactgaaacgtagtaaacccgaaactgagttcgacaatgagttcatcggacctatgatgcgaatggaagcccgtgagaccgcacgcaacgcaaactctacggacctctctcgtatgaacactgataacatgaagtttgaaggtgagcataacggagtaccttatgaggaccttcctactgagaagggcgctgtggtactgcacgatggctccgttataagtgcaaacaacccaatcaaccctaagaccctcaaagagttctctgaggttgaccctgagaaggctgcgcgaggaatcaaacttgctgggttcaccgagataggcttgaagaccttggggtctgacgatgctgacatccgcagagtggctatcgacctcgttcgctctcctactggtatgcagtctggtgcctcagggaagttcggtgcaacagcttctgacatccatgagagacttcacggtaccgaccagcgtacttataatgacttgtacaaagcaatgtctgacgctatgaaagaccctgagttctctactggcggagctaagatgtcccgtgaagaaactcgctacactatctaccgtagagcggcactggctattgagcgtccagagttacagaaggcactcactccgtctgagagaatagttatggacactattaagcgtcactttgacaccaagcgtgaacttatggagaacccagcaatcttcggtaatacgaagtctgttagtattttccctgagagtcgccacaaagggacttacgttcctcacgtttacgaccgtcatgcaaaagcactgatgattcaacgctacggtgccgaaggtttacaggaaggtattgctcgttcgtggatgaacagctacgtctccagacctggggtcaaggtcagagtcgatgagatgcttaaggaattacacggggtgaaggaagtaacaccagagatggtagagaagtacgctatggataaggcttacggtatctcccactcagaccagttcaccaacagttccataatcgaagagaacattgagggcttagtaggtatagagaacaactcattccttgaggcacgtaacttgtttgattcggacctatccatcactatgccagacggacagcaattctcagtgaacgacctaagggacttcgatatgttccgcatcatgccagcgtatgaccgccgtatcaatggtgacatcgctatcatggggtctacagggaaaaccactaaggaacttaaggatgagattttggctctcaaagcgaaagctgagggagacggtaagaagactggcgaggttcatgctctaatggataccgttaagattcttactggtcgtgctcgacgtaatcaggacactgtgtgggaaaccacattacgttccatcaatgacctagggttcttcgctaagaacgcctacatgggtgctcagaacattacggagattgctgggatgattgtcactggtaacgtgcgtgctctcggtcacggtatcccaattctgcgtgacaccctctacaagtctaaaccagtttccgctaaggaactcaaggaactccatgcgtctctgttcggaaaggaggtggaccagttgattagacctcaacgtgctgatatagtgcagcgtctaagggaagcaactgatacagggcctgttgtggcgaacatcgtaggtaccttgaagtattccacacaggaactggctgctcgctctccgtggactaagctgctgaacggaaccactaactaccttctggatgctgcgcgtcaaggtatgcttggggacgttattagtgccaccctaacaggcaagactacccgctgggagaaagaaggcttccttcgtggtgcctccgtaactcctgagcagatggctggcatcaagtctctcatcaaggaacatatggtacgtggtgaggacgggaagtttacagttaaggacaagcaagcgttctctatggacccacgggctatggacttatggcgactggctgacaaggtagctgatgaggcaatgctgcgtccgcataaggtgtccttacaggattcccatgcgttcggagcattaggtaagatggttatgcagtttaagtctttcactatcaagtcccttaactctaagttcctgcgaaccttctatgatggatacaagaacaaccgagcgattgacgctgcgctgagcattatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggcgctcctttggctatggctgacctagttggcggtgtcttagggttcgaatcttccaagatggctcgctcaacgattctgcctaaggacaccatgaaggaacgtgacccaaacaaaccgtacacctctagagaggtaatgggagctatgggttcgaaccttctggagcagatgccttccgctggctttgtggctaacgtaggggctaccttaatgaatgctgctggtgtggttaactcacctaacaaagcaaccgagcaggacttcatgactggattgatgaactctaccaaagagttagtgcctaacgaccctcttactcaacagcttgtggttaagatttatgaggcgaacggtgttaacctgagggagcgtaagaaataatacgactc

SEQ ID NO: 3 - DNA, 32767 bp, synthetic construct; identical to SEQ ID NO: 1
  source 1..32767: mol_type = other DNA; note = Genetically engineered T7 phage where the coding sequence of the tail fiber protein T7p52 is deleted

SEQ ID NO: 4 - DNA, 32767 bp, unidentified
  source 1..32767: mol_type = genomic DNA; note = DSM 112943; Escherichia phage vB_EcoP_JacobBurckhardt (Bas65)
atgttaagcctagactttaacaacgaagtcataaaggcggctccgattgcgggtactgcggttactgatgtggctacccgctacttcttcggattgtcgttaaacgaatggttctatgttgctgctatcgcctacacagtagttcagattggtgctaaggtagtcgataaagtgattgattggaagaaagccaataaggagtagcacatggataaagataagagtctcattacgttcctagagatgttggacactgcgatggctcagcgaatgctggcagacctctcgaatgaagagaagcgtagtcctcaactctacaacgctattaacaaactgttagaccgccacaagttccaaatcggtaagctgcaacctgacgtccacattctaggtggcctcgctggtgctcttgaggagtacaaagagaaagtcggtgacagtggacttacggacgacgacttgtacacgttacagtgacatactcaaggtcctacatattggggcctttatggatgtcattgtctacacgagatgctcctaccgtgaaatctgaaagttaacgggaggcattatgctagaatttttacgtaagctaatcccttgggttctcgctgggatgctattcggattaggatggcatctagggtcatactcaatggacgctaaatggaaacaggaggtacacaatgagtacgttaagagagttgaggctgcgaagagcactcaaagagcaatcgatgcggtatcggctaagtatcaagaagaccttgccgcgctggaagggagcactgataggattatttctgatttgcgtagcgacaataagcggttgcgcgtcagaatcaaaactaccggaacctccgatggtcagtgtggattcgagcctgatggtcgagccgaacttgacgaccgagatgctaagcgtattctcgcagtgacccagaagggtgacgcatggattcgtgcgttacaggatactattcgtgaactgcaacgtaagcaggaaatcaagtaaggaggcaacgtgtctactcaagccaatcgtaatgcgctcgtagtggcgcaactgaaaggagacttcgtggcgttcctgttcgtcttatggaaggcgttaaaccttccggtgcccactaagtgtcagattgacatggctaaggtgctggcgaatggagacaacaagaagttcatcctacaggctttccgtggtatcggtaagtcgttcatcacatgtgcgttcgttgtgtggtccttatggagagaccctcagttgaagatacttatcgtatcagcctctaaggaacgtgcagacgctaactccatctttattaagaacatcattgacctgctgccgttcctcgctgagttaaagccaagacccggacagcgtgactctgtaatcagctttgacgttggcccagctaagcctgaccactctcctagtgtgaaatcagtaggtatcactggtcagttaactggtagccgtgctgacattatcattgcggatgacgttgagattccgtctaacagtgcgactatgggtgcccgtgagaagctatggactctcgttcaggagttcgctgcgttacttaaaccgctgccttcctctcgtgttatctaccttggaacacctcagaccgagatgactctctataaggaacttgaggataaccgtgggtacacaaccattatctggcccgctctgtacccaaggacgcgcgaagagaacctctattactcacaacgtcttgctcctatgttacgcgctgagtacgatgagaaccctgaggtactcgctgggactccgacagaccctgtacgctttgaccgtgatgacctgcgtgagcgtgagctggaatacggtaaggctggctttacgctacagttcatgcttaacccgaacctgagtgatgtcgagaagtaccctctgaggctccgtgacgctatcgtagcggccttagacttagagaaggcaccgatgcattaccagtggcttccgaaccgtcagaacatcattgaggaccttcctaacgttggccttaagggtgatgacctgcatacctaccatgattgctccaacaactcaggtcagtaccaacagaagattctggtcattgacccaagtggtcgcggtaaggacgaaacaggttacgctgtgctgtacacattgaacggttacatctaccttatggaagctggaggtttccgtgatggttactccgataagacccttgagttgctcgctaagaaagccaagcaatggggagtccagacggtcgtctacgagagtaacttcggtgacggtatgtttggtaaggtattcagtcctatccttcttaaacaccacaactgtgcaatggaagagattcgtgctcgtggtatgaaggagatgcgtatttgcgatacccttgagccagtcatgcagactcaccgccttatcattcgtgatgaggtcattagggccgactaccagtccgctcgtgatgtagacggtaagcatgacgttaagtattcactgttctaccagatgacccgtatcactcgtgagaaaggtgctctggctcatgatgaccgattggatgcccttgcgttaggtattgagtatctccgtgagtccatgcagttggattccgttaaggtcgaaggtgaagtgcttgctgacttccttgaggaacacatgatgcgtcctacagtgtctgctaccaacatcattgagatgtctgtaggaggagttgatgtgtactctgaggacgatgagggttacggtacgtctttcattgagtggtgatttatgcattaggactgcatagggattcattattgccaaggaaggcactttttgtacgattcctttagaatcagtaagttagaccctcaaaattaatacgactcactatagggagaggagggacgaaaggttactatatagatactgaatgaatacttatagagtgcataaagtatgcatagtagtgtacctaaagtgacctctaagaatggtgattatattgtattaatatcaccttaacttaaagaccaacataaagggaggaggagactcatgttccgcttattgttgaacctactgcggcatagagtcacctaccgatttcttatggtactttgtgctgcccttgggtacgcatctcttactggagacctcagttcactggagtctaccgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatccgtaaggtctatacctaaagataggccatcctatcggtgtcacctaaagagggtctcagagagggcctgaggagtccctagagggtcctttaaaatataccataaaaatctgagtgactatctcacagtgtacggacctaaagttccccccccataggggcccctaaagcccagccaatcaccttgagccaaccttcggttgaccttgatggtccctaagggttggggttggtcccttggtgttaccttgagtgtctctctgtgtccctatctgttacagtctcctaaagtatcctcctaaagtcacctcctaaagtcattcctaccgtctacagcctaaagactacacttaaagacccattcagtcaatccctatcttaaagataatcctaaagaccatcctaaagaccagcctaaagaccatcataaagcctattcctaaagatgccttgttgttatctataaagagataatcttcatccattgtctttaatacgactcactatagagagagactacttaaagaggccttaaagatggattaatttaaagatttattaaaaagagtattgacttaaagtctaacctataggatacttacagccatcgagagggacacggcgaataaccatccaatcgataccggggtcaaccggataagtagacagcctgataagtcgtatgaaaaacaggtattgacaacatgaagtaacaagtagtaagatatacagcgtcaaccactgacacggtgtcttctaggtgacttaagcgcaccacggcacataaagtgaaacaaaacggttgacaacatgaagtaaacacggtacgatgtaccacacgaaatgactgaacagtgagtcaccacactgaaaggtgatgcggtctaacgaaacctgaccaaagacgctctttaacaatctggtaaatagctcttgagtgcatgactagcggataactcaagggtatcgcaaggtgccctttatgatattcactaacaaccacacatgaggtaacacaagatggctatgtctaacatgacttatcacaacgttttcgatcacgcttacgaaatgctgaaagaaaatatccgttatgatgacatccgtgacgctgatgacctgcacgacgcagtgcacatggcagccgataacgccgttccgcactattacagtgacattttcagtgtaatggcaagcgagggcattgaccttgagttcgaggattctggtctgatgccggacaccaaggacgtaattcgcatcctgcaagctcgcatctatgagcaactcacaatcgacctctgggaagatgcagaagacttactgaacgagtatcttgaggaagtcgaggaagatgaagactacgaagaggatgaagagtaatggctaacaacaccacgcaatacggtctgaccgctcaaactgtactgttttatagtgacatggtgcgctgtggctttaactggtcgctcgcaatggcacaccttaaggaactgtacgagaacaacaaggcaatagctttggaatctgctgagtgatagactcaaggtcgctcaatacgagtggcctttatgattatcattctatgagggttaaacgcatgacgactgaaatctttatggcaaccgttgcagtattcgtagcgatgggatggcgtagcaccgcgttggagcgctcaaagttccttaagcaacgtgatgcactactcgagaagttggacgctgaggggcgcggtatggaagctctggaggcaataaagcactatcgctaattatcatcaccatcggaatcatcaaaggggcactacgcaaatgatgaagcactacgttatgccaatccacacgtccaacggggcaaccgtatgtacacctgacgggttcgcaatgaaacaacgaatcgaacgccttaagcgtgaactacgcatcaaccgcaagattaacaaaatctaatcacatttaattacttggagaatacataaagtgaacatcaccgacatcatgaacgctatcgacgcaatcaaagcactgccaatctgtgaacttgacaaacgtcaaggtatgcttatcgacttgctggtagagatggtcaatagcgagacatgtgatggagagattaccgaactgaatcatgcacttgagtatcaagactggtggactaccttgaagtgtctcacgactgacgcaggtttcaagatgctcggcaatggtcacttctcggctgcttatagtcacccgctgctgcccaacagagtgattaaggtgggctttaagaaagaggactcaggcgctgcgtataccgcattctgccgcatgtatcagggccgtgcaggtatccctaacgtctatgatgtacagcgccacgctggatgttatacggtggtactcgatgcactgaatgattgcgagcgtttcaacaacgatgaacattataaatacgctgagattgcaagtgacatcattgactgcaaatcagatgcgcacgatgggttaactggattggatggcgagttcgtcgagacttgcaagttaatccgcaagttctttgagggcatcgcttcgttcgacatgcacagcgggaacatcatgttcgacgataatgatgtaccatacattaccgacccggtatcattctcacagaagaaagacggtggctcgttcagcattgaccctgaggaactcatcaaggaagtagaggaagtagcacgccagaaggaaattgaccgcgctaaggaacgcaaagcacgccatgtggggcgcttagaggcacgcagaatgaaacgccgtaaccgtaaggcacgtaaagcacaccaagcgaagcacgaaagaatgattgctcgggggagttgggttgaacgtcaagcacgacgtaaccatgagatagctgtagacgtactaggaaggaccaataacgctatgctctgggtcaatatgttagctacggactttaaggcacttgaagaacgaatcgcgatgaaatggcgcaacgctgaccggatggctatcgctaacggtcttccgctcaacatcgataagcaactttacgcaatgctaatgggctgatagtcttatcttacaggtcatctgcgggtggcctgaatagatacgatttactaaccgaaagaggcactaaatgaacacgattaacatcgctaagaacgacttctctgacatcgaactggctgctatcccgttcaatactctggctgaccactatggtgagcgtttagctcgcgaacagttggcccttgagcatgagtcttacgaaatgggtgaagcacgcttccgcaagatgtttgagcgtcaacttaaagctggtgaggttgcggataacgctgccgctaagcctctcattactacactactccctaagatgattgcacgcattaacgattggtttgaggaagtgaaagctaagcgcggcaaacgcccgacagccttccagttcctgcaagaaatcaagccggaagctgtagcgtacatcaccattaagaccactctggcttgcctcactagcgtagacaatacaaccgttcaggctgtagcgagtgcaatcggtcgagccattgaggatgaggcgcgcttcggtcgtatccgtgaccttgaagcgaagcacttcaagaaaaacgttgaggaacagctcaacaagcgcgtggggcacgtctacaagaaagcattcatgcaagttgtcgaggctgacatgctctctaagggtctactaggtggcgaggcgtggtcttcgtggcacaaagaggactccattcacgttggggtgcgctgcattgagatgctcattgagtcaactggaatggttagcttacaccgccaaaacgctggcgtagtcggtcaggactctgagactatcgaactcgcaccggaatacgctgaggctatcgcaacccgtgcgggtgcacttgctggtatctctccgatgttccaaccttgcgtagttcctcctaagccgtggactggcattacaggtggtggctattgggctaacggtcgtcgtcctctggcgctggtgcgtacccacagcaagaaagcactgatgcgctacgaagatgtttacatgcctgaggtatacaaggcgattaacattgcacaaaacaccgcatggaaaatcaacaagaaagtcctagcggtcgccaacgtgataaccaaatggaagcactgccctgtagaggatataccagcgattgaacgtgaagaactcccgatgaaaccggaagacatcgacacgaatccggaggctctcaccgcgtggaaacgtgccgctgccgctgtgtaccgtaaggacaaggctcgaaagtctcgccgtatcagtcttgagttcatgcttgagcaagccaacaagttcgctaaccataaggccatctggttcccttacaacatggactggcgcggtcgcgtttacgctgtgtcaatgttcaacccgcaaggtaacgatatgaccaagggcctgctgacgctggcgaaaggtaaaccaatcggtaaggaaggttactactggctgaaaatccacggtgcaaactgtgctggtgtcgataaggttccgttccctgagcgcatcaagttcattgaggaaaaccacgagaacatcctagcgtgcgctaagtctccactggagaacacttggtgggctgagcaagattctccgttctgcttccttgcgttctgctttgagtacgctggggtacagcaccacggcttaagctacaactgctctcttccgcttgcgttcgatgggtcttgctctggcattcagcacttctccgcgatgctccgtgatgaggtaggtggtcgtgcggttaacttgctgcctagtgaaaccgttcaggacatctacgggattgtcgctaagaaagtcaacgagattctacaagcagatgcaatcaacgggaccgacaacgaagtggttaccgtgaccgatgagaacactggcgaaatctctgagaaagtcaagctgggcaccaaggcgctggctggtcaatggcttgcttacggtgttactcgcagcgtgactaagcgttcagtcatgacgctggcttacgggtccaaagagttcggctttcgtcaacaagtgctggaagataccattcagccagctatcgattccggcaagggtctgatgttcactcaaccgaatcaggctgctggatacatggctaaactgatttgggaagctgtgagcgtgacggtggtagccgctgtggaggcaatgaactggcttaagtctgccgctaagctgctggctgctgaggtcaaagataggaagactggagagattctgcgcaagcgttgcgctgtacattgggtgactcctgattccttcccagtttggcaagaatacaagaagccaattcagactcgcttgaacctgatgttcctcggtcagttccgcttacagccgaccattaacaccaacaaagatagcgagattgacgcacacaagcaggagtccggtatagcacccaattatatacacagtatggacgcaaaccacctgcgacaaaccgtggtgtgggctcatgagaagtatggcatcgagtccttcgcactgattcacgactcctttgggacaattcctgctgatgctgccaacctgttcaaagctgtgcgcgaaactatggttgacacctatgagtcctgtgatgtactggctgacttctatgaccagttcgctgaccagttgcatgagtctcaattggataaaatgccagaacttcctgctaaaggtaacttgaacctccaagacatccttaagtcagacttcgcgtttgcgtaacgtcaaattaatacgactcactatatagggacaaactcaaggtcacttaatgagtggcctttatgattgaccttcttccggttaatacgactcactataggggaaccttaaggtttaactttaagacccttaagtgttaattaaagatttaaattaaagaattactaagagagaggactttaagtatgcgtaacttcgaaaagatgaccaaacgttctaaccgtaacgctcatgacttcgaggcaactaaaggtcgcaagttgaataagactaagcgtgaccgttcccataagcgtagctgggagggtcaataagatgggacgtttatacagtgggaacctgaatgacttcaaggctgcttgcaacaggctctaccagttagactttgctgttatttgtgatgaatggtacgatgcttacaaacgccttgcgtgtatgcgtctacgtgtagaggacagggctggaaatcttattgataccaatacgttctgtcaccacgatgaagacgtcttgtacaatacgtgtactgattggctgaaccggatgcacgaccagttgaaggactggaagtaatacgactcagtatagggacaatgcttaaggtcgctctctgagtggccttagtcattcaaccaaaacatggagataaacattatgatgaacattaagaccaacccgtttaaagcaacgtctttcgtagagtctgccattaagaaggctcttgataacgctgggtatcttatcgccgacattaagtacgatggtgtacgtgggaacatctgcgtagacgatactgctaacagttgctggctctctcgtgtatctaaaacgattccggcactggagcacttaaacggattcgatgttcgctggaagcgtctgctgaacgatgaccgttgcttctacaaagatggctttatgctcgatggggaactcatggtcaagggagtagactttaacactgggtccggcctactgcgtaccaagtggctaaacaagaagaactttgagttcgacgttggcggtgtgccaccacttaaggggtccaaggttgagttcgagttggacaccaaacgtctgagtgtccgactgtacgctatcctcccgcttcacatcgtggagtctggggaagaccatgatgtcatgacgttactcatgcaggaacacgttaagaacatgctgcctctgctacaggaatacttccctgaaatcgaatggcaagcggctgagtcttacgaggtttacgacatggtggaactacagcaactgtacgagaagaagcgagcggaaggccatgagggtctcattgtgaaagacccgatgtgcatctacaagcgcggtaagaagtccggctggttcaagctcaagccagagaacgaggctgatggtatcatccaaggtgtgaattggggtacagaaggtctagccaacgaaggtaaggtaatagggttcacggtgcttcttgagacagggcgtattgtagacgctaacaatatctctcgcgccttaatggacgaatttaccgccaacgttaaggagcacggggaggactactataacggatggtcttgccaagtaaattatatggaggaaacaaaagatggaagccttagacacccttccttcacctgcttccgaggggttgagggaaatcgttgcgaaaagatttaaggttaacagggaggctcctagcggcctctctcgcgttagcgggaaaccagtcggctacaagaagtctagcggatactggaatatccacgtttccgttaacaacaagtctcaccatctacttgcgcatagggttatatgggaactcacttacggagagataccctctggaatgcagatagaccacattaatagagacaggtctgataatcgcatagagaaccttagactcgttactatgacagagaatatgcacaataagggagactacgtgaccaacactaccggtcataaatacgtcatgtatcgtaaagataggaatgcgtacatattcagaataaagcgatttggcaaactacatacttcataccacaagactttggatgaggctgtggaggccaagcggttgttccttaatcagtaaccacactggctcaccttcgggtgggcctttctgcgtttaatacgactcactaaaggaggtacacaccatgatgtacttaatgccattactcattgtcatcataggatgccttgcgctccactacagcgatgacgatatgccagatggtcacgcttgacaacacccgctaaggagacaccctatgatgtacttaatgccattactgatgctattcttggtgtcattactcattctcatccctgtatatgctgtactacgtgatgatgacgatgatgacacacaagatgaccatgcttaatacgactcactaaaggagacactatatgtttcgacttcattacaacaaaagcattaagaatttcacggttcgccgtgctgaccgttcaatcgtatgtgcgagcgaacgccgagctaaattacctcttattggtaacacagttcctctggcaccgagcgtccacatcattatcacccgtggtgactttgagaaagcaatagacaagaaacgtccggttcttagtgtggcagtgacccgcttcccgttcgtccgtctgttactcaaacgtatcaaggaggtgttctgatgggatgcatagcgtgcttagagaaagacgaccgttatccacatacctgtaataaaggagctaacgatatgaccgaacgtgaacaagagatgatcattaagttgatagacaataacgaaggtcgcccagatgatttgaatggctgcggacttctctgctctaatgtcccttgtcacctctgccccgcaaataacgatcaaaagataaccctaggtgaaatccgagcgatggacccacgtaagcctaaactggagaaacctgaggtaactactacagatgaccagccttccgctaagtcaatcgaaggtgtcactaagccttcccactacatgctgtttgacgacattgaggctatcgaagtgattgctcgttcaatgactcgtgagcagttcaaaggttattgctttgggaatatcttaaagtacagactacgtgctggtaagaagtccgaactagcttacttagagaaagacatggcgaaagctgggttctacggtgaactgtacgagaaacataaggataagtgctatgatgcttaagccttccgaatggtgccacaagatgtggactgaatgtatagaacgtggtgacgaggaatctgctggtcactacatggaactttacaaccaatggctatcgagaggttactgatatgtcaaacgtaaatacaggttcacttagcgtggacaataagaagttttgggctaccgtagagtcctctgagcattcctttgaggttccaatctacgcggagaccctagacgaagctgtggagttagccgagtggcaatacgttccggctggctttgaggttactcgtgtgcgaccttgtgtagcaccgaagtaatacgactcactattagggaagactccctttgagaaaccaaatgaaacctaaaggagattaacattatggctaagaagattttcacctctgcgctgggtactgccgaaccttacgcttacatcgctaagccggactacggtaacgaagagcgtggctttgggaaccctcgcggtgtctacaaagttgacctgactattcccaacaaagacccgcgctgccagcgtatggtcgatgaaatcgtgaagtgtcacgaagaggcttatgctgctgccgttgaggaatatgaagcgaacccgcctgctgtagctcgtggtaagaaaccgctgaaaccatacgaaggcgacatgccgttcttcgataacggtgacggtacgactacctttaagttcaaatgctacgcgtctttccaagacaagaagaccaaagagaccaagcacatcaatctggttgtggttgactccaaaggtaagaagatggaagacgttccgattatcggcggaggctctaagctgaaagtcaaatactctctggttccgtacaagtggaacactgctgtaggtgcgagcgttaagctgcaactggaatccgtgatgctggtcgaactggctacctttggtggtggtgaagacgattgggctgacgaagttgaagagaacggctatgttgcctctggttctgccaaagcgagcaaaccacgcgacgaagaaagctgggacgaagacgacgaaggctcttacgaagaagactcagacggagatttctaagtggctggcgcatacgctgcgcgtggtatccgaaaggtcggggcattccgttccggcctagaagataaggtctctaagcagctagagagtaaaggtatacagtttgattacgagcagtggaagattccctacgtagtccctgcgagtaatcatacctattgcccagattttctactgcccaatggtatcttcatcgagaccaaagggctatgggaaagtgatgaccgcaagaagcatctcctgattcgcgagcagatgcctgagctggatatacgacttgtgttctcaagctcccgcacaaaactctacaaagggtctccaacgtcctatgcagaattttgtgaaaagcacggcatcaagtttgctgataagctgattccagttgagtggctcaaagagcctaaacgggaggtgccattcgataagctcaaacagtcaaaaggagctaagaagtgattcatccaaaactcttgatagagccgaccgattcctgtattgaatggacaggcgcgttccacaagaacgggtacggagtaatctctttgaatcgcaaagtggctgccgagctaggaatacctagggtgcaatttgtacacagggcaagctacatgcagcataaaggaactataggtgatggacttgttgttcgtcacttgtgtcacaacagaaagtgtttcaacccagctcacttggaagtcggcactcagtacgataacttcgaagatagtgttaaggttgggcgcaacaaacgtaaactgcttgatgaggacgttaaatcaattagagagtccacaatgagtaaccgcaagttagccaagctatacggtgttagctctacaacgatcttccacatcaagcacaacaacaagtggaggaatttaaatggcacgagtacaattcaaacagcgtgaaagcaccgatgcaatcttcgttcattgctcagcaacaaaacctaatatgaattggggtgtacgtgagattcgccagtcgcataaagagcgtggattcctagatgtgggataccactttatcatcaagcgtgatggtactgtagagaccggtcgagacgagatggctgtaggctctcacgctaagggctacaatcacaactctattggtgtctgccttgtaggtggcatcgacgataaaggtaagtttgacgctaactttacgccagcccaaatgcaatccctccgctcgctgcttgtcacacttctggctaagtacgaaggcgctgtgcttcgcgcccatcacgatgtggcacctaaggcttgcccttcgttcgaccttaagcgttggtgggaaactaacgaactggtcacttctgaccgtggataattaattgaactcactaaagggagaccacaacggtttccctttgttcgcattgttgattaaggaataaccatatggaattagaacaagatagtattttcctatatcatgctccttgcgagaactgtgggtcttctgatggtaactctgtgtattctgacggtcatgaatactgcttcgtatgtgagcaccgagttccggcctcagacgaacgtaaggaaaagttatcatcaagaagacgtatagtcggtggaggtaaaccaatgtcatacaacgtgtggaacttcggggaatccaatggacgttactctgcgctaactgctaggggaatctcaaaagagacttgccaaaaagctggatattggataggtaaggttgacggtgtgatgtaccaagtggctgactatcgtgaccagaacggcaacattgtgagccagaaggttcgcgataaagataagaacttcaagacaactgggagtcacaagagcgatgccctgttcgggaagcacctgtggaacggtggtaagaagattgtcgtaaccgaaggtgaaatcgacatgcttaccgtgatggaacttcaagactgcaagtatcctgtagtgtcgttgggtcacggtgcctctgctgctaagaagacatgcgctgccaactacgaatactttgaccagttcgaacagattatcttgatgttcgatatggacgaagctggacgcaaggcagtcgaagaggccgcacaggttctacctgctggtaaggtacgagtggcagttcttccgtgtaaggatgccaatgagtgtcatcttaatggtcatgaccgtgaaatcatggagcaagtgtggaacgctggcccttggattcctgatggtgtggtatcggctctttcgttacgagaacgaatccgtgaacacctgtcgtcagaggaatcagtgggtctacttttcagcggctgcacaggtatcaacgataagacattaggtgcccgtggtggtgaagtcattatggtcacttccggttccggtatgggtaagtcaacgttcgtccgtcaacaagctctccagtggggcacagcgatgggtaagaaggtaggcttagcgatgcttgaggagtcagttgaggagaccgctgaggaccttataggtctacacaatcgtgtccgactgagacaatccgactcactaaagagagagattattgagaacggtaagttcgaccaatggttcgatgaactattcggtaacgatacgttccatctatatgactcattcgccgaggctgagactgatagactgctagctaagctggcctacatgcgctcaggcttaggctgtgacgtaatcattctagaccacatctcaatcgtcgtgtcagcttctggtgaatccgatgagcgtaagatgattgacaaccttatgaccaagctcaaagggttcgctaagtcaactggggtggtgctggtcgtaatctgtcaccttaagaacccggacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggttacatggaatacaacaaggaaaccggatggcttgaaccctcaagttactcaggggaagaagagtcacactcagagtcaacagactggtccaacgacactgacttctaacaggattcttgatgactttccagccgactacgagaagtttcgctggagagtcccgttttaatacgactcactaaaggagacacccatgttcaaactgattaagaaattaggccaactgctggttcgtatgtacaacgtggaagcaaaacgtctgaacgatgaggctcgtaaagaggccacacagtcacgcgctcaggcaattcgctccaacgaactggctgatagtgcatccactaaagttacagaagctgcccgtgtagccaaccaagctcaacagctttccaaattctttgagtaatcaaacaggagaaaccattatgtctaacgtagctgaaactatccgtctatccgacaccgctgaccagtggaaccgtcgagtccacatcaacgtgcgcaacggtaaggcgactatggtttaccgctggaaggactctaagtcctctaagaatcacactcaacgtatgacgttgaccgatgagcaagcactgcgtctggtaaatgcgcttaccaaagctgccgtgacagcaatccatgaagctggtcgcgtcaatgaagctatggctatcctagacaagattgatagctaaagagtggtatactcaaggtcgcccaagtggtggccttcatgaatactattcgactcactataggagatattatcatgcgtgaccctaaagttatccaagcagaaatcgctaaactggaagctgaacttgaggacgttaagtaccatgaggccaagactcgctccgctgttcacattttgaagaacttaggttggacttggacaagacagactggctggaagaagccggaagtttacaagttgagccacaaggtgtttgataaggacactatgacccacatcaaggctggtgattgggttaaggttgacatgggagttgttggtggatacggctatgttcgctcagtgagtggcaaatacgcacaagtatcgtacatcactggtgtcactccacgaggtgcaatcgtagccgataagaccaacatgatccacacaggattcttgactgttgtctcgtatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagctacaattgaaccgagagttccacattcctcgtgagaactgtatcgacacccttgtgttgtcacgcttgattcattccaacctcaaggacaccgatatgggtcttctgcgttccggcaagttgcccggaaaacgctttgggtctcacgctctggaggcgtggggttatcgcttaggcgagatgaagggtgaatacaaagatgactttaagcgtatgcttgaagagcaaggtgaagaatacgttgacggaatggagtggtggaacttcaacgaagagatgatggcctataacgttcaggacgttgtggtgactaaggctctacttgagaagttaatctctgacaaacactacttccccgaccaaggtatcgactggactgacgtaggtaacacattctggcgtgagtctgtagaggccgtcgagctagagcataaagctgcgtggctgctcgctaagcaggaacgtaacggctttccattcaataccaaagctatcgaagagttgtacgtagagttggctgcccgtcgctctgagttgctccgtaagctgaccgaaacgttcggctcatggtatcaacctaaaggtggcactgagatgttctgccatccacggacaggtaagccactacctaaataccctcgcattaagacacctaaagttggtggcatctttaagaagcctaagaacaaggcacagcgtgaaggccgtgagccttgcgaactggatacccgcgagtatgttgctggtgcaccttacactccagtagagcacgtggtgtttaacccttcgtctcgtgaccacattcagaagaaactccaagaggctggatgggtcccgaccaagtacaccgataagggtgctcctgtggtggacgatgaggtactcgaaggagtgcgtgtagatgaccctgagaagcaggctgcaatcgacctcattaaagagtacctgatgattcagaagcgaatcggacagtctgctgagggagacaaggcatggctccgttacgttgccgaggacggtaagattcatggttctgttaatcccaacggcgctgttactggtcgtgctactcatgctttccctaacctagcacagatacccggtgtccgttctccttacggggagcagtgtcgcgctgcttttggcgctgagcaccatttggatgggctaactggtaagccttgggttcaggcaggtatcgacgcatccggccttgagttgcgctgcttggcccactttatggctcgctttgataacggtgagtacgctcacgagattcttaatggcgacatccatactaagaaccagatggctgctgaattgcctacccgtgataacgctaagacgttcatctatgggttcctctatggtgctggtgatgagaagattgggcagattgttggtgctggtaaagagcgcggtaaggaactcaagaagaaattccttgagaacacccccgcgattgcagcactccgcgagtctatccaacagacacttgtcgagtcctctcagtgggtagcaggtgaacagcaagtcaagtggaaacgccgctggattaaaggtctggatggtcgtaaggtacacgtccgtagtccgcatgctgccttgaataccctactgcaatctgctggtgctctcatctgcaaactgtggattatcaagaccgaagagatgctcgtagagaaaggcttgaagcatggctgggatggggactttgcgtacatggcatgggtacacgatgaaatccaagtaggttgccgtaccaaagagattgctcaggtggttattgataccgcacaagaggcaatgcgctgggttggagaccactggaacttccggtgtcttctggataccgaaggtaagatgggtcctaactgggccgtatgtcactgattctataggagaaattattatggctatgaccaagaaatttaaagtatcattcgacgttaccgctgtaatcactagcgaaggtgaacgaaacatacaagactgcgtcatgcgagttgccaaggcagtagccgctggtgaacaagttgacgactttaagaaagagttgcttgtccaagcgttgacgaatggtcctgagggcctcgctacgttcctgattaagcatggacttcgctcattcgttaaggatagctacaatgacctgagcgagtctgagcgtaaactgattcgactctctcctgcaactgtacgggaggtgttctgatgtctgactatctgaaagtgctgcaagcaatcaaaggttgccctaagactttccagtccaactatgtacggaacaatgcgagcctcgtagctgaggccgcttcccgtggtcacatctcgtgtctgaccacaagtggacgtaacggtggcgcttgggaaattaccgcttctggtactcgatttctgaaacgaatgggaggttgtgtctaatgtctcgtgaccttgttactattccccgcgatgtgtggaacgatatgcagggctacatcgactctctggaacgtgagaacgatagccttaagaatcaactaatggaagctgacgaatacgtagcggaactagaggagaaacttaatggcacttcttgaccttaaacagttctacgagttacgggaaggctgtaacgacaaaggtatccttgtgatggatggcgactggctggtcttccaagcgatgagtgctgctgagtttgatgcctcatgggaggaagagatttggcaccgctgctgtgaccacgctaaggcccgtcagattcttgaggattccatcaagtcctacgagacccgcaagaaggcttgggtaggtgctccgattgtccttgcgttcaccgatagcgttaactggcgtaaagaactggttgacccgaactacaaggctaaccgtaaggccaccaagaaacctgtaggttactttgagttccttgaggccctctttgagcgcgaagagttctattgcatccgtgagcctatgcttgaaggcgatgacgttatgggagttattgcttccaatccgtctgcattcggtgcgcgtaaggctgtaatcatctcgtgtgataaggactttaagaccatccctaactgtgacttcctgtggtgtactactggtaacatcctgactcagacagaagagtccgctgactggtggcacctcttccagacaatcaagggtgacatcaccgatggttactcagggattgccggatggggtgatactgctgaggacttcttgaataacccgttcattaccgagcctaaaacgtctgtgcttaagtctgggaagaacaaaggccaagaggttactaaatgggttaaacgtgaccctgagccacatgagacgctttgggactgcattaagtccattggtgctaaggctggtatgaccgaagaggatgttatcaaacagggccagatggctcgcatcctacggttcaacgagtacaactttattgacaaggagatttacctgtggagaccgtagcgtatatcggcattggtctttgtggtctcggagtgtgcctcattgcgtggggcatttgggacctagccagaataatcaagttgatacacgacactaagtgataaactcaaggtccctaaattaatacgactcactatagggagataggggcctttatgattattactttaagatttaaccctaagaggaatctttatcatgttaacacctattaaccaattacttaagaaccctaacgatattccagatgtacctcgtgcaaccgctgagtatctacaggttcgattcaactacgcgtacctcgaagcgtctggtcatataggacttatgcgtgctaatggttgtagtgaagcccacatcttgggtttcattcagggcctacagtatgcctctaacgtcattgacgagattgagttacgcaaggaacaactaatagatgatggggaggattgaccttatgtgtttctcaccgaaagttaaagttccgaagatggataccaatcagattcgagccgttgagccagcgccactgacccaagaagtgtctggcgtagagttcggtgggtcttctgatgagacggataccgaaggcaccgaagtgtctggacgcaaagacctaaaggtcgaacgcgatgattccgtagcgaagtctaaagccagtggtactggttccgcccgtatgaaatcttcaatccgtaagtccgcatttggaggcaagaagtgatgtctgagttcacatgtgtggaagctgagagtcgcttccgtgcaatccggtggactgtagagcaccttgggttgcctaaaggattcgaaggacactttgtgggctacagcctctacgtagatgaagtaatggacatgtccggttgtcgtgaagagtacattctggactctaccggaaaacatgtagcgtacttcgcgtggtgcgtaagctgtgatattcaccacaagggagacattctggatataacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagtcaagaaggccgtgaagaaagtcactaagtccgttaagaaagtcgttaaggaagtggctcgtccggttaaacagattgctggtggtctggctggtggcgctggtgaagcacaggtagttgaagttccacaagctgccgcaactcctgctgcacagattgttgacgtacctgagaaagaggtttccactgaggacgaagcacagaccgaaagcggacgtaagaaagctcgcgctggtggtaagaagtccttgagtgtagcccgtagctccggtggcggcatcaatatttaatcaggaggtgacacatggctgagaagcgtaaaggtctagccgaggaaggtgcaaagtcggtatatgaacgattgaagaacgaccgcgctccgtatgagactcgtgctcaaaactgcgctcagtacaccattccgtccctcttccctaaggattccgacaactcatccacagactacacaactccgtggcaagctgtgggtgctcgcggtctcaataacttggcctctaagctcatgctggctctcttcccgatgcaaacgtggatgaaactgaccatctctgagtatgaggctaaacagttactgaatgaccccgatggactcgctaaggtcgatgagggtctctcgatggtagagcgtatcatcatgaactacattgagtctaacagttaccgagtgactctctttgaggctctcaaacagttagtcgtagctggtaacgtcctgctgtacctaccggaaccggaagggtcaaactataatcccatgaagctgtaccgattgtcttcttatgtggtccaacgagacgcatttggcaacgttctgcaaatggtgactcgtgaccagatagctttcggtgctctccctgaggacatccgtaaggctgtagaaggcaaaggtggagagaagaaagctgatgagacaatcgatgtgtacactcacatctatctggacgaagactctggtgagtatgtccgatatgaagaggtagagggcatggaggttcaaggctccgatgggacttaccctaaagaggcttgcccatacatcccgattcggatggttcgactggacggagaatcctacggtcgttcgtacattgaggagtacttaggtgacttacggtcccttgagaacctccaagaggctatcgtcaagatgtcaatgattagctctaaggttatcggcttagtgaatcctgctggtataacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgctgttcagcgcacaggtgaacgtgtgaccgccgaagagattcggtatgtcgcttctgaacttgaagatactttaggtggtgtctactctatcctttctcaagaactacaattgcctctggtacgagtgctcttgaagcaactacaagccacgcaacagattcctgagttacctaaggaagccgtagagccaaccattagtacaggtctggaagcaatcggtcgtggtcaagaccttgataagctggagcgttgtgtcactgcgtgggctgctcttgctcctatgcgtgatgaccctgacatcaaccttgcaatgattaagctgcgtattgccaacgctatcggcattgacacttctggcattctactcaccgaagaacagaagcaacagaagatggtccaacagtctacgcaaatgggtatggataacggtgcttctgcgctggctcaaggcatggctgcacaggctacagcctcacctgaggctatggttgctgccgctgattccgtaggtctacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgaacagaacatgctggctcttgatgttgctgcccgtgatggcgatgatgcaatcgagttagcgtccgacgaagtggaaacagaacgtgacctgtatgacaactctgacccattcggtcaagaggacgatgagggccgcattcaggttcgtattggtgatggctctgagccgaccgatgtggacactggagaagaaggctttgagggcaccgaaggttccgaagaatttaccccactgggcgagactccagaagaactggtagctgcatctgagcaacttggcgagcacgaagagggcttccaagagatgattaacattgctgccgagcggggcatgagcgtcgagaccattgaggccatccagcgtgagtacgaggagaacgaagagttgtccgctgagtcctacgctaagctgtctgaaattggctacacgaaggctttcattgactcgtatatccgtggtcaagaagctctggtggagcagtacgtaaacagtgtcattgagtacgctggtggtcgtgaacgttttgacgcactgtataatcaccttgagacgcacaaccctgaggctgcacagtcgctggacaatgcgttgaccaatcgtgacttagcgacagttaaagccatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagaccctgaaattaatacgactcactatagggagaccacaacggtttccctctagaaattattctttgtttaactttcttaagaaggagacatacatatggctaacatgactggtggacagcaaatgggtactaaccaaggtaaaggcgtaagtactggttctgccgataaactggcgttgttcttgaaggtatttggcggtgaagttctgactgcattcgctcgtacctctgtgaccacttctcgccacatggtccgctccatctccagcggcaaatctgctcagttccctgttctgggtcgcactcaggcagcgtatctggctccgggtgagaacctcgacgataaacgtaaggacatcaaacacaccgagaaggtaatcaccattgatggtctcctgacggctgacgttctgatttacgatattgaggacgcgatgaaccactatgacgttcgctctgagtacacctctcagttaggtgaatctctggcgatggctgcggacggtgctgttctggctgagattgctggtctgtgtaacttgacgagcacccataacgagaacattgagggcttaggttccgcaactgtaatttcaaccactcagaacaaggcagcacttaccgaccaagttgcgctgggtaaggagattattgcagctctgacgaaggctcgtgcggctctgactaagaactacgttcctactgctgaccgtgtgttctactgcgacccagatagctactctgcgattctggcagcactgatgccgaacgctgcaaactacgctgctctgattgaccctgagaagggttctatccgaaacgttatgggctttgaggttgtagaagttcctcacctcactgctggtggcgctggtactaagcgtgatgggacaaatgaaggtcagaagcactctttcccagctgaatacgcgagtggtaccgctgaggtagctaaggacaacgttatcggcctgttcatgcatcgttctgcggttggtactgtgaagctgcgtgacttggcgctggaacgcgctcgtcgagctaacttccaagctgaccaaatcatcgcgaagtacgcaatgggtcacggtggcctgcgcccagaagctgctggtgctgtggttttcgcacaagcgtaatgcaagaagtgacctcaacggtcgctgctagtaccgaagaggtaagcattactgccgaagagagtctaacgccagcacagaaggctgcacgcactcgcgctgctaacaaagcccgaaaggaagctgagttggctgctgccactgctgagcaataactagcatacccccttggggcctctaaacgggtcttgaggggttttttgcttaaaggaggaactatatgcgctcatacgatatgaacgttgagactgccgctgagttatccgctgtgaacgacattctggcatctatcggtgaacctccggtatcaacgctggaaggtgacgctaacgcagatgtagcgaacgctcggcgtattctcaacaagattaaccgtcagattcagtctcgtggctggacgttcaacattgaggaaggcataacgctattacctgatgtttactccaacctgattgtatacagtgacgattatctgtccctaatggctacttccggtcaatcaatctacgttaaccgaggtggctatgtgtatgaccgaacgagtcaatcagaccgctttgactctggtattactgtgaacattatccgtctccgagactacgatgagatgcctgagtgtttccgttactggattgtcactaaggcttcccgtcagttcaacaaccgattctttggggcaccggaagtagagggtgtacttcaagaagaggaagatgaggctcgacgtctctgcatggagtacgaagtggactacggtgggtacaatatgctggatggtgatgcgttcacttctggtctactgactcgctaataatcaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctctggttttccttaagacactgggaggtagtggcgacttaggacaagcaccttacattcacctgattaaccgtgatgagaacgaacagtattacgctgtgttcactggtacgggaatccgtgtgtttgaccttgccggtaacgagaagcaagttaggtatcctaacggttccaactacatccagaccgctcagccgcgtaacgacctgcgaatggtcactgtagcagactatacgttcatcgttaaccgtaacgttgttgtacagaaggacccactttatatcaacttaccgaattataaccctaagcaggacgctctgataaacgttcgcggtggtcagtatggtcgtaccttacagattatgattaacggtggtacacaagcctcctatcagattcctgatggttcacagccagcacacgttaataacacggacgctcagtggttagcagaagagttggctcgccagtgtagagcaaatgctccgggatggaccttcaatgttgggcagggttttatccacatcgtagcaccttctggtcaacagattgattccctgatgactaaggatggctatgctgaccaattgattaaccctgttacccactacgctcagtcgttctctaagttgccacctaatgcccctaacggctacatggtgaaaatcgtaggggacgcctctaagtctgccgaccagtattacgttaggtatgacgctgagcggaaagtttgggctgagactttaggttggaacactgaggaccaagttctatggaacaccatgccacacgctcttgtgcgagcctctgatggtaatttcgacttcaagtggcttgagtggtctcctaagtcttgtggtgacgttgatactaacccttggccttcttttgttggttcaagtatcaacgatgtgttcttcttccgtaaccgtttaggtttccttagtggtgagaatatcatccttagtagaacagctaagtattttaacttctacccagcgtcaatcgcaaacttgagtgatgatgacccaatcgacgtagctgtaagtaccaaccgaatcgcaatccttaagtatgccgttccgttctccgaagagttactcatctggtcagacgaagcgcaatttgtcttgacggcatctggtacgcttactgcacgttccgttgagttgaacctaacgactcagtttgacgtacaggaccgagcgagaccttttgggattgggcgtaatgtttactttgctagtccgaggtccagcttcacgtccatccacaggtactacgctgtgcaggatgtaagctccgttaagaacgctgaggacattacagcacacgttcctaactacatccctaatggtgtgttcagtatctgcggtagcggtacggaaaacttctgttcggtattatctaacggggacccaagtaaaatcttcatgtacaaattcctgtatctgaacgaagagttaaggcaacagtcgtggtctcattgggactttggggaaaacgtacaggttctagcgtgtcagagtatcggctcagatatgtatgttattcttcgcaatgagttcaatacgttcctagctagaatctctttcactaagaacgccattgacttacgaggcgaaccttaccgtgcctttatggacatgaagattcgctacacgattccgagtggaacatacaacgatgacacgttcactacctctatccatattccaaccatttatggtgcaaacttcgggagggacaaaatcaccgtattggagcctgatggtaagataaccgtatgtgagcaaccaactggtgggtggaacagtgacccttggctgagactcagcggtaacttagagggacgtgaagtgttcattgggttcaacatccacttcgtctatgagttctctaagttcctcatcaagcaaactgccgacgatgggtctacctccacggaagacattgggcgattacagttacgccgagcgtgggtgaactatgaggattctggagcgttcgacatttatgttgagaaccaatcgtccaactggaagtacacaatggctggtgcccgcttaggctctaacagtctgagggcaggtcggctgaacttagggaccggacaataccgattccctgtggtgggcaacgctaagttcaataccgtatacatcgtgtcagatgcgactacccctctgaacatcattgggtgtggctgggaaggtaactacttacggagaagttccggtatttaattaaatgttctccctgtggtggctcgaaattaatacgactcactatagggagaacaatacgactacgggagggttttcttatgatgactataagacctactaaaagtaaagactttgaggtattcactccggctcaccatgacattcttgaagctaaggctgctggtattgagccgagtttccctgatgcttccgagtgtgtcacgttgagcctctatggattccctctagctatcggtggtaactacggggaccagtgctggttcgtaacgagcgaccaagtgtggagacttagtggaaaagctaagcgagagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcaattattcacaataacgaaaggaggataaccatatgtgttgggcagccgcaattcctatcgctatagctggcgctcaggctatcagtggtcagaacgctcaggcaaaaatgattgccgctcagaccgctgctggtcgtcgtcaagctatggaaatcatgaggcagacgaacatccagaatgctgacctatcgttgcaagctcgaagtaaacttgaggaagcgtcctctgagttgacctcacagaacatgcagaaggtccaagctatggggtcaatccgaacggctatcggagagagtatgctcgaaggtgcctcaatggaccgcattaagcgagtcacagaaggacagttcattcgggaggccaatatggtaactgagaactatcgccgtgactaccaagcaatcttcgcacagcaacttggtggcactcaaagtgctgcaagccagattgacgaaatctacaagagcgagcagaaacagaagagtaagctacagatggttctggacccgctggctgtcatggggtcttccgctgcgagtgcttacgcatccggtgcgttcgactctaagtccaaaactaaggcacctattgttgccgctaaaggaaccaagacggggaggtaatgagctatgagtaaaattgaatctgcccttcaagcggcacaacctggactctctcggttacgtggtggggctggcggtatgggctatcgtgcagcaaccactcaagctgaacagccaaagtccagcctattggacaccattggtcggttcgctaaggctggtgccgatatgtataccgctaaggaacaacgagcgcgtgacctagctgatgaacgctctaacgagattatccgtaagttgacccctgagcaacgccgagaggctctcaacaacgggacccttctgtatcaggatgacccatacgctatggaagcactacgagtaaagactggtcgtaacgcagcgtaccttgtggacgatgacgttatgcagaaggtcaaagagggtgtcttccgtactcgcgaagagatggaagagtatcgtcacagtcgtcttcaagagggcgctaaggcatacgctgagcagttcggtattgaccctgaggacgttgattatcagcgtggtttcaacggtgacattaccgagcgtaacatctcactgtatggcgcacacgataacttcttgagccagcaagctcagaagggtgccatcatgaacagccgagtggaactcaacggtgtccttcaagaccctgatatgctgcgtcgtccagactctgcggacttctttgagaagtacatcgacaacggtctggttactggagcaatcccgtctgacgctcaggctacacagcttataagccaagcgttcagtgacgcttctagccgtgctggtggtgctgacttcctgatgcgagttggtgacaagaaggtaacactcaacggagccactacgacttaccgagagttgatgggtgaggaacagtggaatgctctaatggtgacagcacaacgctctcagtttgagactgacgctaagctgaacgagcagtatcgcttgaagattaactctgcgctgaaccaagaggaccctcgtactgcgtgggagatgcttcaaggtatcaaggctgaactagataaggtccaacctgatgagcagatgacaccacaacgtgagtggctaatctccgcacaggaacaagttcagaaccagatgaacgcatggacaaaagctcaggccaaggctctggacgactccatgaagtcaatgaacaaacttgacgtaatcgacaagcaattccagaagcgaatcaacggagagtgggtctcaacggactttaaggatatgccagtcaacgagaacactggtgagtttaagcatagcgatatggtaaactacgccaataagaaactcgctgagattgaccgtatggacattccagatgctgccaaggacgccatgaagttgaagtaccttcaagcggactctaaggacggtgcattccgtacagcaatcgggactatggtaacagatgctggtcaagagtggtctgccgctgtgattaacggtaagttaccggacaataccccagctttggatgctctacgcagaatccgcaatgctgaccctcagttgattgccgctctgtatcctgaccaagctgagttattcctgacgatggacatgatggacaagcagggtctcgaccctcagcttcttcttgacgctgaccgactgactgttaagcggtccaaagagcaacgctttgaggacgataaagcattcgagtatgcactgaatgcatctaaggctcctgagattgcccgtatgccagcgtcactgcgcgaatctgcacgtaagatttacgactcagttaagtatcgctcagggaacgaaagtatggctatggagcagatgaccaagttccttcaggaatctacctacacgttcactggtgatgatgttgatggtgataccattggtgtgattcctaagaatatgatgcaggttaactctgacccgaaatcatgggagcaaggtcgggatattctggaggaagcgcgtaagggaatc

SEQ ID NO: 5 - DNA, 32767 bp, unidentified
  source 1..32767: mol_type = genomic DNA; note = DSM 23252
tctcatagttcaagaacccaaagtaccccccatagccctcttaaagccaccacctaaagccagccctacccctctggtttaaccttcggttaaccttgggtatctcctcaagtggcctataggatagcctaaagtggttcctaaagtcaatacctaaagtgatggatggcttaaagaggccttaaagagtgtcctagtgtaccagttcgatagtacatccctatgtgtccctatctgttagccacccttaaagtaaaaccctaaagccaccacctaaagccaccacctaaagagtggagctatcttaaagacaaccagaccgatagacccgctgttaacctgtagtgatgccttgtagttatctgtagtcatttcattaaccctcactaaagggataaggtggatacttaaagaggaataagggatttaataaaagattaaaattaagtgttgactttaagttacctttaaggctattattcatctcgtcaacgggacaacggccttaaagccactccctgagataccggaggttaaccggataagtagacagcctgataagtcatacgaaaaacaggcattaaccgaaagtagtacaacaagttatcttctagtcaaacaaaagcgttgacacggtaacgagaaagcagtaggatgtatcgcagtagggtcgctatcggctcccacggtagcagttcgggaaggcactacagcctaacaagtggttgaccaccacgagacattaagcgcccattagtgggaaccacgcggagtcaaacggtcgcccactgaggggctacagctcacgaatcaggttgacaagctgataataacgcagtaagatgtaccacattgaaacgacagtgaggcgggacactgaaagccgctagggtctaacgaaaccagaccgacacgctctttaacaatctggataatagctcttgagtgtaccgtataagcgaataactcaaggtcgcactgaaagcgtggcctttatgatattcacttaacgaactaacatgaggtaacaccaaatgattttcactaaagagcctgctaacgtcttctatgtactggtgtctgctttccgttccaacctcgatgatgaggtgaacatgagtcgccatcgccacatggtaagcactttacgtgccgcaccgggtctttatggctccgttgaatcaaccgatttgaccgggtgctatcgtgaggcaatcgcaagcgcaccgactgaggaaaaaacggttcgtgtacgctgcaaggacaaggcgcaggcactcaatgttgcacgcttagcttgcaatgagtgggagcaagattgcgtactggtatacaaatcacagactcacacggctggtctggtgtacgctaaaggaatcgacggttacaaggctgaacgtctgccgggtagcttccaagaagtgcctaaaggcgcaccgcttcaaggttgcttcacgattgatgagttcggtcgccgctggcaagtacagtaagtgttaaactcaaggtcgctccatgcgagtggcctttatgattatcacttataggactaacaccatgagtaaacttctggcaacatctaaaatagaaggccaatgcactgtaacactgcgtgagtattaccacgggtcaatgggaagcacctacgttgtgcgatacggtaaacaggtcacacattgggtaaacccgattttagcacaagaggattatcaatcttgtgtgttacaccaaacaacatgcgcagggtggaatgactaatgactatgatgacgatatttgtactcactgtgtacactgcgattgtggtgtatttcctgagagactttaagaacggtctggtagtccataaagcaacactgagctactttaagtggggattcatcccacggtttacggttcgtgaacgcaacggacggtttaaggccaacaaggtaggagtattctacattgctacgcactaactcaaagcacgtaaagaccgcactgtatgcgatggcgtatggtgcatcgaaacgcaaggtcaaacgcatcttaacgagacaccgcaagatgaccgcacgacaggctgcaagcgctgtcaaatgggctgaatttattctttactcttcttacagataacaggacactgaacgatgactttcaccgaagcacaagccacactcgaagctatccgcaacctgccgattgaccgcttggatgaacgccagcccatgctggtggctcttgctgctgccattgtgaacgctgagacccaagatgggcaactgacgcaggacgcaggaaacaaggcgctcgaacatatggactactggcagacgctggcggagcgcatgagggatgttggtttcatcaagttaggaaatggtcactttagcgcagcatatgagcacgcattgttaccgggcaaggttatcaaggtaggctttaagaaagaggactcaggcgctgcatacgctgcgttctgtcgaatgaatcaaggccgggaaggtatccctacggtgtacgatattcagcgccatgctggttgttacacggtggttcttgactatctgaacccgcttaacgattggtgctacggtgagagtgacaaggtgcgtcaccacttcaaagccgcacgggaaatcattgaggaggattgctttgatgctgccgaagagtacccaataagtgatgggttcattgagacctgtaaggatatccgcaagttcttcttaggcatcgcatcgttcgattgtcacagcgggaacatgatgaaagaccagatgggacgcctgattatcacagacccggtatcattcagcgcagacgacaagttaaagccgggtgagttccactgtgacccggatgaactcatcgctgagcttgaggcgctgagagcgcaggaagcgattgaccgctgtaaggcacgtaaggcacgccgtgaccctaaaggagactttcaggcagcacgtaaagcccgtgccaaatggcgcagaaaggcctctaagcgtgataagcgcaacgctaaggtactcgctaagatgcgcatggatgccaacgagcaacgcagggccgagcctagggcgcgtatgttgtggggtgataagcactggcgcaacgcttggatgcagcacatgaatgttgacttcgcacggctcgaactgttagcagccgcacagatggcacagatagaccacaaggctatcgcatggaacgctccgctgtgtgtcgataagtaccttgacaggagactgcaaggctgatagagtcttttcttacaggtcatcatgtggtggcctgaataggaacgatttattcacaatgaggtaagcaatgaacatcatcgaaaacatcgaaaagaatgacttctccgaaatcgaactggctgctatcccgttcaacacactggctgaccactacggcagtgcattggctcgtgaacaactggctttagagcatgagtcttatgagctaggcgagcgccgcttcctcaagatgcttgagcgtcaagcgaaagctggtgagattgcagacaacgcagccgctaagccattactcgctacgctccttcctaagctaaccacacgcatcgttgagtggctagaagagtacgccacgaagaaaggccgcaagcctgtagcctatgcaccactccaatcactcaagccggaggcgtccgcgtttatcacactcaaggtcatccttgcgtcactgaccagcacgaacatgacaaccattcaggccgctgctggtatgcttgggaaggccattgaggatgaggcacgtttcgggcgcatccgtgacctcgaagcgaagcacttcaagaagcacgttgaggaacagcttaacaagcgccacgggcaagtctacaagaaagcctttatgcaggtggttgaggcagatatgattggtcgcgggctgctgggaggtgaggcgtggtctagctgggacaaggaaaccacgatgcacgtagggattcgcctgattgaaatgctgattgaatccacgggtctggtggaattacagcgccacaatgcaggtaacgcaggctctgaccatgaggcactgcaactggcccaagagtacgtggacgtattagcgaagcgtgcaggcgctctggcgggtatctctccgatgttccagccgtgtgtcgtcccgccgaaaccttgggtagcaatcaccgggggcggctattgggctaacggtcgcagacctttggcactcgttcgcactcactctaagaagggactgatgcgctatgaggatgtttacatgccagaagtttacaaggcggtcaacatcgcacagaacaccgcatggaaaatcaacaagaaagttcttgctgttgtcaatgagattgttaactggaagaattgcccggtcgcagacattccatcgctggagcgccaagagttaccgcctaagccggacgacattgacaccaacgaggcagcactcaaggagtggaagaaagccgccgctggtatataccgcttggacaaggcacgagtgtctcgacgtatcagcttagagttcatgctggagcaggccaacaagttcgcaagcaagaaagcaatctggttcccttacaacatggactggcgcggtcgtgtgtacgctgtgccgatgttcaacccgcaaggcaacgacatgacgaaaggtctgctgaccctcgctaaaggcaagccaatcggtgaggaagggttctactggctgaaaatccacggtgcgaactgtgcgggtgtcgataaggttcctttccctgagcgcatcgcgttcattgagaagcacgtagacgacattctggcttgcgctaaagacccaatcaataacacttggtgggccgagcaggattcacctttctgtttcctagcgttctgcttcgagtatgcaggcgttgcgcaccacggtctgagctacaattgctccctgccgctggcgttcgacgggtcttgctctggtatccagcacttctccgcgatgctacgcgatgaggtaggcggtcgtgcggttaacctgctgccaagcgaaaccgtgcaggacatttacggcatcgttgcgcagaaagtaaacgagattctcaaacaggatgcaatcaacggcacacctaacgagatgattaccgtgaccgataaggacaccggggaaatctctgagaagctcaagctgggaacctcaacgctggcgcaacagtggctggcatatggtgtaacccgtagcgtaactaaacgttcggtcatgacgctggcttacggttccaaggagttcggctttcgtcaacaggtactggacgacaccattcagcctgcgattgacagcggtaagggcttgatgttcacccaacctaaccaagcggctggctatatggctaagctgatttgggacgcggtgagcgtgaccgtagtcgcagcggttgaggcgatgaactggctcaagtctgccgctaagctgctggctgctgaggtcaaggacaagaagaccaaggagattctgcgccaccgttgcgcggttcactggactacaccggacggcttcccggtctggcaggaataccgcaagccactacagaagcgtctcgatatgattttcttagggcaattccgtctgcaaccgacgattaataccctcaaggattcaggcattgacgcacacaagcaggagtctggcatcgcgcccaactttgttcactcacaggatggtagccacctccgcatgacagtcgtttatgctcacgagaattatggcattgagtcctttgcgctcatccatgacagctttgggactatcccggcagacgctggtaagctctttaaggctgtgcgtgaaacgatggttatcacctatgagaacaacgatgtgctggcagacttctatgaccagtttgcagaccagctacacgagacccaactggacaagatgcctcctcttccgaagaaagggaacctgaacctgcaagacattctcaagtctgactttgcctttgcataacaagcacttagcattaaccctcactaacgggagactacttaaggtctcccactttaagacactttaggtactaagagattaaatttatgattaacattaagactttctttaagaacatctttaagttaaaccgcttgacttccgttaagttctacgcttggatgcccggtagcgatgacctccgcaagactgaatttaagttaggcttagggccgtgcggtaaggtggtcacgaagttggaatgttactcaacgtcctccgggatggttatcttccagaccactgaggacaacgaaacgaaatccttctactacccgaaaggttcaacgtgtggtcgtatcgaacggacatacagttaaccctcactaacgggagagttaaacttaaggtcatcaccgacggtggcctttgtgattaactttcaatacacatcaacatgaggtaagatactatgcgtaccaactttgagaaatttaccaagcgcgattcagtggtcaacgagcatggcgaacagtggcaagagcgccgtgaccgcatgaagaaacgccacaagcaacagcgcggtaactcacagaaacgggagtggaactaataatgatgggacgtatctatagcggcaacctgaacgattacaaagatgcggtagcgcgtctacaggaagaccatgacgtgaccgtgaaggtggagtcattcagctatgaaaacccagcgaagatgtgcaggtcatccggtgaggttctccgtgtgttcacacgctccgggcatccggtggcatccagaaccttcgagcacagcgacagtgatgtacaaatcaacgcgcagactgcatggctccgtaaggttcacagcgatttgaaacactggaagtaataaccctcactaacaggagaatccttaaggtcacttaacatgagtggcctttgtgattcgaactgattaactttaaaccaatgaggaacaaccgtatgaacatcttcaagaccaacccattcaaagctgtgtcattcgtagagtctgccgttaagaaagcgctggagacctccggctacctgattgcggacattaagtatgatggcgtgcgtggtaacattgtggttgacaacgtggctgaggccgcatggctgtcccgtgtgtccaagttcatcccggcgctggaacatctgaacggtttcgataagcgctggcaacaactgatgaatgatgaccgctgcattttccctgatggcttcatgcttgatggtgaacttatggtaaagggcgtagacttcaacacagggtctggcctactgcgaaccaagtgggtgaagcgtgacaacatggagttccacatcacgaacgtcccgacgaagctgacccagaaaggccgcgaagttatcgacggtaactttgagtttcaccttgaccctaagcgcctgatcgtgcgcctgtatgcggtcatgccgattcacattgcggagtctggcgaggactacgatgtgcagaacctgctgatgccatatcatgtggaagccatgcgctctcttctggttgaatacttcccggaaatcgagtggcttatcgctgagacctacgaggtctacgatatggattcactgagtgaactatacgaggccaagcgtgcagaaggtcacgagggtctcattgtgaaagaccctcaaggcatctataagcgaggaaagaagtctggctggtggaaatgcaagcctaatgacgaagccgatggtattattcagggtgtcaattggggtacgccgggtcttgcaaacgaaggtaaagttatcgggttctctgtgcttcttgagacgggtcgtctggttgacgccaacaacatctctcgcgcattgatggatgagtttaccgcgaatgttaaagaacatggcgaggattactacaacgggtgggcctgccagattggatatatggaggaaacacctgatggctcattgcgacacccctcattcacctgcttccgaggaactgaggacaatccacaagagaaagtataagttagaccgggaggctccttcgggagtcgtttccgcctctggtaaacctttagggtataagcggaaagatggctactggattctctcttatggtggcaaacaaatgctggcacatcgtgtggtctggatgttgacccacggtgaaatcccggaaggtatggtcattgaccacatcaacaggaaccctagtgataaccgtatcgaaaaccttcggtgtgtcactcaagctgttaacttatcgaatgttgacgtccggtctcattgtttaagtggcgagaagtatattgcccttgacaagagaaccggacgttttgccgttcgtatcaggcgcaaatcccacggaacctatggaacccttgaggaagctatagccgtgcgagatgctgccattaaccctcactaacaggagacacacaccatgtggattatcctattcgctatcgtcgcaacgctgggactaatggttgctgacgacaacatttggcctgattgttaaggagacaacatcatgcgtttacacttcaacacatcaaatggtatcttttcggttcgccgtgaagaccgctctacagcagtggcctctgagcgcaacgccaagctgccgctgattggttcggtcgtcccattgtcgccgcgtgttcacctactgatcactcgtggagagttcattaaggcgatgaacaaagagcgcccacatctggaagccgtggtcacttactggcctcgcatccgcctgttcgttaagtggattaaggaggtgctgtaatgttacaggaatattatgatatgcgtagccgcttcccggttaactctgccgttcgttattcaggagcgaaccctttgttcaagggtctgaccggaacagtgcagggctactctcacactggtcgagtcaaggttcgcttcggtgttcgtgaggctgaggtacatccggccatactaatcccgctaccgaaagtcggcccgaacgtggaagcaccgaagacagaagtcaagtctgacgtaacccaccctaaccactacatgctgttcgacaacgtggaagccattgaggttatcgcccggtcaatgaccgtcgaggcgttccgtgggtactgcttagggaacatcctaaaataccggctgcgtgctggtaagaaatccgaactggcaacgatggagaaagaccttaagaaagcggctttctatcaggaactctttgacaaacaccgggggctgtgttatgacgcttcgtgaatgctgcgattggtgtgccgctaagtggaaccaagctattgaggctggtgacgaggccgcagccaacgcctaccaacaattgtacgcgctgtgggagcggcgctttaaggaggccaataaatgagccagtgtaacaacgggtgccagtacgcgaaggacgtagggatggagaaccactcctgtgcaaatcgctgcatgtactcagaacagaaataccttctgacgatagaaggcaacaccgagtcctttgaggttctggtattcgcccggtctctcgaagaggcaacgctacaggctgaacattatgaggacgctgggttcgtagtgactcgcatccgtccagaagtgaaagcctaattaccctcactaaagggaacaacccaatcattaaccacaaaggagaaacactatggctggtttcaagaagaaaatctacacctctggtcttggcactgctgagccttatgcttacctgagtaagccggattatggcaacgaagagcgtggcttcggcaacccacgtggtgtctataaggtagacctgactctttccaataaagacccgcgctgtcaggcgatggtcgatgaaatcgtgaagactcacgaagaggcttatgctgctgccgtggaagagttcgaagctaacccgccgcaggtacagcgtggtaagaaaccactgaaaccttacgagggcgacatgccgttcttcgataacggtgacggcacgactaccttcaagttcaaatgctatgcgtctttccaagataagaagaccaaagagaccaaacacatcaatctggtcgtggtcgatagcaaaggtaagaagattcaggaagtgcctattatcggtggcggctccaagctgaaagtgaaatactctctggtgccttacaagtggaacaccgctgtgggcgcaagcgtcaagctgcaactggagtccgtgatgctggtcgaactggctaccttcggtggtggcggtgaagatgagtgggccgatgaagtagaagatggtggttacactgcgagtgaatcccgtcagtcccgcgatgagcaggagtggcaggaagacgagcacgaagaaaccccggatgaggaagaggacttctaatggctggcgcatacgctgcgcgaggtgtacgcaaggtcggggctttccgctccggcctagaagataaagtctcaaagcagcttgagagtaagggaattaagttcgactatgaactttggcgtatcccttacgtcatccctgcgagtgaccacttatacactccagacttcctgctacctaatggcatcttcattgagaccaaggggctgtgggattccgatgaccgcaagaaacaccttttgattcgtgagcaacaccctgaactggatattcgcttggtcttctcaagcagccgctcaaagctctacaaaggtagcccaacaagctacgccgagtggtgtgagaagcatggcattctgtttgctgacaagctaattccggtggagtggctcaaagaacccaagaaggaggttccgttcgataaatttaagactaagaaaggagtaaagaaaaatggctaaagttcaattcaaaccacgagcaaccacggaggcaatctttgtgcattgctcagcaaccaagccaagccagaacattggcgttcgtgagattcgtcagtggcacaaagagcagggctggttagacgtaggatatcacttcatcatcaagcgtgatggcactgtggaagcaggccgcgatgaactggctgtaggttcccacgtgaaaggttacaaccacaactccgtaggcgtatgcctcgtgggtgggattgatgataaaggcaagttcgacgccaactttacacctgcgcaaatgcaagcgctgcgtagtctgctggtcacgctgctggcgaagtatgagggttcaatccttcgtgctcaccatgacgttgcacccaaagcctgcccgtccttcgacttgaagcgctggtgggagaagaacgaactggttacatctgaccgagggtgataccatgaggatgatttgtattcgctgtgataagccgttgaagagtcgtgtaccgtcacgtcagtgtcagtgcaaagacccaatcaaggcggaggaacacaccgaggaataattaacactcactaaagggagacttaacggtttccctttgttcgcactattgattaaggattaaccacatggaacaagaaaacgacagtatattcctttaccatatcccgtgcgagaactgtgggtcttctgatgctaactcattgttctccgatggtcacacgtactgttacgcctgtgagaattggacgccgggggatgaacagaaagctgagcagttatcaacgagaagacgtacaggaggcagcaagcctatgagctatgacgtatggaatttcggtgacagcaacggtcgatactctgacctgactgcccgtggcatctcgaaggagacatgccaaaaggctggctactggctggcgaaagtggacaaccgaatgtatcaggttgccgactaccgagaccagaacggctccatcgtgtcacagaaggtgcgcgataaggacaagaactttaagaccaccggaagccacaagagcgatgcgttgttcttgaagcacctctggtctggcggtaagaaaattgtggtcactgagggcgaaattgatgcgctcaccgtgatggaactccaagactgtaagtacccggtcgtatcattgggtcacggtgcctctgctgctaagaagacatgcgccgctaactatgaatactttgaccagttcgagcagattatcttgatgttcgacatggatgacgcaggccgtaaggcagtcgaagaggccgcacaggttctcccggctggcaaggttcgtgttgctgtgttaccatacaaggacgccaatgagtgtcacatcatgggtgaagacaaagcaatcttggagcaggtctggaacgcgaacccttgggtgcctgatggtgttgtctctgcgttgtcactcaaagaccgcgttaaggaagctatgacctccgaggacgctgtaggtttattattcgatggctgtcaaggcctgaacgatagaaccttgggtgcccgtggaggcgaagtcatcatggtcacttccggttccggtatgggtaagtcaacgttcgttcgtcaacaggcgctggcttggggcaagcgaatgggtaaacgtgttggcctagcgatgctggaggaatccgttgaggataccattcaggacatgatgggcttgaacaataaggttcgtctgcgtcagtctgatgaagtcaagaaggctatcgcagaggacggacgtttcgatgaatggtatgatgaactatttggagacgatacgttccacctttatgactcctttgcggaggccgaagctgacagactattagcgaagctggcctacatgcgaacaggcttagggtgtgatgttatagtgctcgaccacatctcaatcgtcgtgtctgcctctgaggaatcggatgagcgtaagatgattgaccgcctcatgactaagctaaaagggttcgctaagtcaactggtgttgtgctcgtagtgatttgtcacttgaagaacccggagaagggaaaagcacatgaagaaggacgcgctgtttctattactgacctacgcggttctggtgccctgcgccagctttctgatactatcatcgccttggagcgtaaccagcaaggtgatatgccacatctggttcttgtgcgtttgcttaagtgtcgtttcactggcgatactggaattgctgggtacatggagtacaacagagaaacaggatggcttgaaccgtctagctatactggcgaagaaggagaaggagatactggctggactgaacaagacggacagtcagacttctaaaggatgcccgtgtgggcattccgattgcgatggaatcatgtaccactaattaaccctcactaacgggaacaacctcaaaccataggagacacaccatgtttaaattcattaacgctttaggcaagctggtagttaagctgtacttcatcgaagccaagaagctggacaagaaggccaaagctgattcacagcgagccattgagttggcgaaacagtcccgcgagaaatccgatgcggctgttagtggcatccacaagtcggcagcgattgcagccaaagcacagtccatgagcaaattctttgagtaaggagcgcacactatgagcaagtcaatcactcacgcaaacaccatccgtctgccagacaccgctgaccagttctcccgccgagtacacatcaatgtgcgcggcgagaaggtcacgatggtctaccgctggaaagaccacaagtctccgaaagcacacactcaacgaatgaccttggatgacaagcaggtcggtcgcttgatgggtgccctgactatggctgctgacaaggtggtcggtgataaccgtgagcgtctggtagagttcggcgctggtatgcaagagattatcgagaagtgataaactcaaggtcgcccaagggtggcctttatgattatcatttagcacgaaaccaaaggagggcattatgctcgtaagtgacattgaggccaacaacctcttagagaaagtcaccaagtttcactgtggcgttatctatgactaccgtgatggtgagtatcatagttatcgtccgggcgacttcggggcgtatctggatgcgctggaagccgaggtgaagcgaggtgggcttattgttttccataacggtcacaagtatgacgttccggctttagagaaactcgcaaagctgcaactcaatcgtgacttcaagttaccccgtgagaactgtattgatacattggtactctcgcgtttacttcatgcgaaccttaaagacacagacatggggcttcttcgttccgggaaactacccggtcgtcgcttcgggtctcacgctcttgaggcatggggttatcgcttgggcgagatgaaaggtgaatacaaggacgacttcaaggctatgctggaagagcagggcgaagaatatgttgacggtgctgagtgggtttcgttcaacgagccgatgatggcgtataacgttcaagacgttgtggtcactgtcaagctcttagagaaattccttacagacctccactacttccctgcgggtatggacttcacgaagtacgatgcggatttattctggcgtgaagctggcgagtctgtggatattgaacatcgtgcagcgtggctgttagcgaagcaggagcgtaacggcttcccgttcaacacacaggccattgaggaactttatgtagaactggcagctaaacgcgcagaacttctgcgaaagttaaccgaaacgtttggctcgtggtatcaaccgaaaggtggtaaggaactcttcaagcacccacggacaggcaaaccacttccgagttatcctcgcgttgtctatccgaaacaaggtggcgtgtacaagaagcctcgtaataaggcacagcgcgaaggtcttgaaccttgcgacttggacacgagggattacatggagggtgctccgtatactccggtcgagttcgttacgttcaagccaagtagccgtgaccacattcagaagaaactacaggaggccggatgggttcctgagaagttcaccgagaagggtgcgcctgtggtggacgatgaggtcttggagcatgtacgagtggatgaccctgagaagcaggcgtgcattgacttgattaaagagtacctgatgatacagaagcgtatcggtcaggcggcagaaggtgacaaagcgtggttacgttatgttcaagatgatggtaagattcatggagccgttaaccctaacggtgcagttactggtcgtgctactcatagtttcccaaaccttgcacaagtacccggcgttcgttctccttacggggagcagtgtcgtagcgcttttggtgctgaacatcatttggatgggattactggcaagccgtgggtgcaagcggggatagacgccagtggcttagagttgcgctgtctggcacacttcatggctcgtttcgataacggagagtatgcgaatgagattctcaacggtgacattcacaccaagaaccagaacgctgcggaactcccaacgcgagacaacgccaagaccttcatttacgggttcctttacggagcaggagatgagaagattggtcagattgttggggcagggaaggagcgcgggaaggaactcaagaagaaattcttagagaacaccccggcgattgcagcgctacgagaagctatccaacagtctctcgttaattcttccgcttggattggtggtgagcagaaagtccagtggaaacgccgttggattaaaggtctggatggtcgcaaggttcacgtcaggtctccacacgcagcgctcaacacattgttgcagtctgctggtgctctgatttgtaagctgtggattatcaagaccgaagagatgctcattgagaaaggcttgaagcatggctgggatggtgactttgcgcagatgggatggattcatgatgaagtccagattgcgtgtcgcaccgaagaggtagccaaggtcgtcgtagaggtagctcaagaggctatgcgatgggttggtgaacattggaacttccggtgtcgcttggacacagaaggtaagattggccctaactgggcagtttgtcactaaggaggatttatgggaggcgtggctacactaaggttccacaagatgccgaccgggtgtgtagtctgcgtttcacataagcgcaatcacgatggctacttccggtacagtatagggtcgtccagaaagggcgagaagaaatgctttatgtttcaccgctgggtttgggaacgacaccgtgggcctataccagaagggtacgagatagaccacttgtgtctgaaccgtgggtgctgcaacgtagaacacttgcagtgcatcccgaagcgtgagcacataatcaaaaccaacaaagagcgcaagctcgtaaaggagaaacattatggcaatgaccaaacactttaaagtaaccttcgacgttaccgctgtgattgacagtgaatccgagaagaatctgaacgagttgctggtagacttagctaagaaggccggggctggcgagaagctgaatccgttcaaacaagaactgcttattcaaggtctgacacacgggccggaaggtgctgctgcgttctgtatcaagcaaggcctccgtgagtttatccgcgatggtcacagtgagttaagctctaccgagcagaagctgatgagattctcacctgcaactgtgcgagaggtgttcaaatgagtgaatacctgaaagttctggcagctatcaaaggttgccctaagtcctttcagtcgaactatgtgcgaaacaatgcggctctcgtagctgaggccgcttctcgtggacacattagctgtttgactgtgggcggtcgcaatggtggagcatgggaggtcacagcctctggtgtgcgcttcctgaaacgaatgggaggttgcgtatgattatgcctaagagtgacaccgtaacgatgacccgtgatgcgtggaacgatgttagcgcgtacattgataagctggagaaagacctagagttcctgaatgcgcttaaggcgtgtggtgtggataattgggacggatactctgatgcagtcgaaatggtttacggggaggatgacgaatgagcctaatcacattgaaagacttcgcggagatgcgtgaaggtaagcctatggagaagggagtgctggtcatggatggtgactggttggtatatcagtcaatggctgctgccgaagttgagacggattggggagacgacatttggactcttgagtgtgaccacgccaaggcacggagcattctcgattctgcaatcgaatcgtaccgaacccgtaagaaggcttggagtgacgctatggttgttctggcgtttaccgacgatgtgaactggcgtaaggtactggttgatgagacctataaggagaaccgcaaggcgacccgtaagcctgtgggctaccgtgacttcctgtcgaagctgtgggagcgcgatgagttcattcacatcaaagaggacatgctggaaggtgatgacgttatgggcatcattgggtctggtcacgaggtcttcggcttcaagaaggctgtgttggtctcctgtgacaaggacttcaagactatcccggacgttgacttcctgtggtgtaccactggtaacatcttgacgcagaccaaagagaccgctgattggtggcatctcttccagaccatcaagggtgatatgaccgatggctactccgggattcccggctggggcgatacggcagaagccttcctgaatgacccgttcattgtggagcctgtagagtccgtgttgaagtctggtaagaacaaggggcagacagtaaccaagtgggtgaagcgtgctcctgatgcgactgagacgctgtgggactgcattaaatccattggtgccaaagctgggatgaccgagcaggaaatcatcaagcaaggccagatggctcgcattcttcgcttcgaggaatacaattacattgacaaggagatttacttatggacgccgcgaagctgattaccatcgctgtgctggtaggtatgctggcgcgaggtctctggatgttcgctctcatagtccgagacgtgaagcgagactccgagattaagtgataaactcaaggctctctgtattaaccctcactaaagggaagagggagcctttatgattattactttaagacttaaccttaagaggtgaaattatgttacaaccaattaatcatgttttaactcatcccgatgatattcctagtatgcctcgtgcagctaaagagtatctacaggtgcgcttcaatcagacctatgtgatggagtccggggagtaccgtgcgctacgcgctgctggatactctgagtctttcattgcgggtgtgatgcatggtctgtaccttgcctctcgaacacttgatgagattgaggtgcgtaaagagcaactgagacaggagtaattatatgtgctggaaaccgaaggtgaaagtccctaagatggacactaaccagattcgcgctgccgaaccagcacctttgactgaaccacctaaatctgtggtatggggaggcgatgacgatgaggacaccagcgtgtcttcctctgaggttccaaccacacagagcagcggtaagagcagtcttaaggtaaagctggatgacagcgcagccaagaacaagagtaaatcctccattcgctccaaggcgtttgggtaactgagtcatagcatagggagaaacatcatgggattcttcaagaaaatcaagaaggctgttaagaaagttgtgaaggaagttagcagaccagtcgagaaggcaggtaaggaagttggtaaggttgttggaggcgcgctgggtgcaggtaagcaggagattatccaacaagaggctccagcaccagtagtagctgcaccgccaccagcacagattgtagacgtacctgaacaggataaggctgagggcgaggatgaggcgcaaaccgagagcgcacgtaagaaggcacgcgctggaggtaagaaggcacttagcgtagctcgcagttccggcggtggaatcaatatctgaggaggactgaatggctgattcaaaacgtacaggattgggcgaagacggtgctaaagctacctatgaccgcctaacgaacgaccgtagagcctatgagactcgtgcggaaaactgtgcgcaatacaccattccgtccttgttcccgaaggagtccgataacgaatctaccgactacacgactccgtggcaggctgtaggtgcgcggggtcttaacaatctggcctctaaattaatgctggcactattcccgatgcagtcgtggatgaagctgaccattagtgaatatgaggcgaagcagcttgttggagaccctgatggactcgctaaggtggacgaaggtctgtctatggttgagcgtattatcatgaactatatcgaatccaacagttaccgtgtaacactctttgagtgcctcaagcagttgatcgtggcaggcaatgcactgctttacttaccggaacccgaaggtagctacaatccgatgaagctgtaccgattgtcttcttatgttgtccaaagagacgcatacggcaatgtgttacagattgtcactcgtgaccagatagcctttggtgctctcccggaagacgttcggtctgcggtagagaaagctggtggtgagaagaagatggacgaaatggtcgatgtgtacacccatgtgtatctcgatgaagagtccggtgattacctcaagtacgaggaagtagaggacgttgagattgatggctccgatgccacctatccgactgacgcgatgccctacattccggttcgcatggttcgcattgatggtgagtcttacggtcgctcctactgtgaagaatacttaggtgacttaaggtcgcttgagaatctccaagaggctatcgttaagatgagcatgattagtgcgaaggtcatcggtctggtgaacccggctggcatcacgcagccccgtagattaaccaaagctcagactggtgacttcgttcccggtcgtcgagaagacattgacttcctgcaactggagaagcaagctgactttaccgtagcgaaagctgtgagtgaccagatagaagcacgcttatcgtatgcctttatgttgaactctgcggtacaacgaacaggtgaacgtgtgaccgccgaagagattcgatatgttgcgtcagaactggaagatacgcttggtggcgtctattcgattctgtctcaagaactgcaattgcctctggttcgtgtgctcttgaagcaactccaagcaacctcgcagattcctgagttaccgaaagaagccgttgagcctactatcagcacaggtctggaagcaatcggtcgtggtcaagacctcgataagctggagcgctgtatctccgcttgggctgctctggctcctatgcagggagacccggacattaaccttgctgtcattaagctgcgcattgctaacgccattggtattgatacttctggtatcctactgacggacgagcagaagcaagcactgatgatgcaggatgcggcccaaacaggtatcgagaacgctgccgccactggtggggctggtgtgggtgctctagcgacctcaagtccagaagctatgcagggtgcagcagcacaggctggcctagacgccacctaataaccctcactaaagggagacctcatctttgaaatgagcgatgactaaaggttggagtcctttggtttccctttatctttaacaacttaggagatttaacaatggctgaatctaatgcagacgtttatgcgtccttcggtgtgaacaacgcggtaatgaccggaagtacacctactgaacacgaacagaatatgctgagtctcgatgttgctgcccgtgatggcgatgatgcaatcgtacttagcgaggaaccgagttcccataacgatgacccatatgcatccggtgtagacccgttcgctgagggcgaggatgatgagggccgtattcaggttcgtatcagtgaagatggtagtgaagctgagttcgacactggtagcgataacgctgaggtggagaccgaaggtgaggccgctgagtttgaaccgctgggcgacactccagaagaactgagccaagtgactgaacaattaggtcagcacgaagaaggctttcaggcgatggtcgagcaggccgttgagcgtggactgagcgcagagtctgtgactcgcatctacgaagagtacgaagccgatggtatctctgagaaatcctacgcggaactcgaagctgctggctatagtcgtgcctttgtggactcctacatctccggtcaggaagcccttgtagaccagtacgtcaatcaggtagttgcctttgctggtggtaaggagcgctttagcgcaatccatacgcaccttgaagcaaccaacccggctgcggctgagtcccttgagactgccatgatgaaccgagacttggcgactgtaaaagcaattatcaatctggctggtgagagttacacgaagaaattcggtaagcctgccaaccgtagtgttaccaagcgtgctactccggttaaacctgtagctcgtcagaaagagggctttacgaatcaggctgagatgattaaagctatgagtgacccgcgttaccgtagtgatgctgcctaccgtcaaatggtagaacagaaggttatcgactccagtttctaattaaccctcactaaagggagagaccatagatgactacaatggttgaatcacctgagcacagaactttgtggtcactcccgtaggtgaaacattgagaaccaactcgattcaagtagtaaccaaccttttctttaaattaacataaggagattcaacatggctaacattcaaggcggacagcaaattggtactaatcagggtaagggtcagtccgcagcggacaaattggcgctgttcctgaaagtgttcggcggtgaagtcctgacggctttcgctcgcacctccgtgaccatgcctcgtcacatgctgcgctctattgcttctggtaagtccgcacaattccctgtgattggtcgtaccaaagccgcttacctgaaaccgggtgagaacctcgatgacaaacgtaaggatatcaaacacaccgagaaggtaatccacattgatggcctgctgactgcggacgttctgatttacgacattgaggacgcgatgaaccactacgacgttcgcgctgagtacaccgcacagttgggtgaatctctggcgatggcggccgacggtgctgtactggctgaacttgctggtctggttaacctgcctgacgcctccaatgagaacatcgagggtctcggtaagcctaccgtattgactctggttaagccgaccactggcgacctgactgacccggttgagctgggtaaagcgattatcgctcagttgactatcgctcgtgcatctctgaccaagaactacgttccggctgctgaccgcaccttctacaccactcctgataactactccgcgattctggctgccctgatgccgaacgctgcaaactatcaggcactgattgaccatgagcgcggtactatccgtaacgtgatgggcttcgaagtggttgaggttccgcacctgaccgctggtggcgctggtgatacccgtgaggatgccccggctgaccagaagcacgctttcccggcgacttccagcactaccgttaaggttgctctggataacgttgtgggcctgttccagcaccgctctgcggttggtacggtcaagctgaaagacttggctctggagcgcgctcgtcgtgcgaactatcaggctgaccagattatcgctaagtacgcgatgggtcacggcggtctgcgtccagaagctgctggcgctatcgtgctcccaaaggtgtcggagtaattcccgaccccactggagttacgttaagtcaaaagaccatgacgcttgttgagggtgcatcccgtgccctcacaggaacagtccagccttccgatgcgaatcggtcgttaagctggtcatcttctgcggaagatgtggctgtatgggaagctggaaaggttgtagccaaaggtgcgggaacagcggacattacagctactacctctaatggcctgaccgcttcctgtaaagtgactgttaacgccgcaacgtcttaaaacacgtagtgcccaattatggcatcatgtggcgttaagcactctcaagccaataagtggcacatacgtctgactatggggactaccaagagctacgactatcatgtgggcaaccaccctgaaagctcgttgtgattgggataacaatctactaatatgcaaaccccttgggttccctctttgggagtctgaggggttttttgctttaaccctcactaacaggaggtaacatcatgcgctcttatgagatgaacattgagaccgcagaagagctatcagccgtcaacgacattctggcttccatcggtgagccaccagtatcgacccttgagggtgatgcaaacgctgatgttgcaaatgctcgacgtgtgctcaacaagattaaccgacagattcagtcacgaggatggacattcaatattgaggaaggtgtgactcttctaccggatgcgttctctggtatgattccatttagctctgactatctgtccgtaatggcaaccagcggtcagacccaatatatcaaccgtggtggctatctctatgaccgctctgcgaagactgaccgatttccttctggtgttcaggtaaacctgattcgtctgagagagttcgatgagatgcctgagtgcttccgtaactacatcgtcaccaaggcttcacgccagttcaacaaccgattcttcggtgcgccggaggttgatggggtgctccaagaagaagaacaggaagcgtggcgtgcgtgcttcgagtacgaactagactacggcaactacaacatgctggacggtgacgcattcacctctggtctacttaaccgctaataacaaggaggctctatggctctcattagtcaatcaatcaagaacctcaagggtggtatcagccaacagccagatattctccggttcgctgaacaaggtagcgtacagattaacggttggtcttctgagtccgagggtctccagaagcgcccaccgatgattcaccttaagacccttggccctgctgggtatgtgggtgcgcaaccttacgtgcatcttatcaaccgtgatgagttcgagcagtatttcgttgtgttcactggtgaagacattaaggtgttcgacctcgatggtaaggagtaccaagtacgtggagaccgctcatatgttcgtacagctaacccacgagaagaccttcggatgataactgtggctgattatacctttgtgactaaccgcaaggtggttgtgcagagtaacgaccaatcggtcaaccttccgggctttaaagaccaaggcgatgcgctgattaatgttcgtggtggacaatatgggcgtagactctcaatcgagttcaacggggctgagcgtgctgctgttcaactaccagacggctcacaaccagcacacgtcaatgaggttgacggtcaggccatcgctgagaagctggcggctcagttgcggaataacctagggaaccccaacaatgaccaagacccaaataagtggcggttcaatgttggccccggattcatccatatccttgcgccaaataacgataacgtttggggactacagactaaggatggttacgcagaccagctaatcaaccctgttacccactacactcagtcgttccagaagctgcctattaacgcgccagatgggtacattgtgaagattgtaggtgacacctcaaagactgccgaccagtattacgttaggttcgacctcaaccgtaaggtgtgggtagaaacaattggatggaacacaaggactcacctatattaccacacgatgccttgggcgctggtacgtgcttctgacgggaactttgacttcaaggtacttgagtggggtgctcgtactgtgggtgatgatacaaccaacccatacccatcgtttaccggacagacaatcaacgatattttcttcttccgtaaccgtttaggcttccttagtggggagaacatcatcttgtctcgcacctcgaaatacttcaacttcttcccggcatccgtgtcaaactactccgacgatgacccaatcgacgttgcggttagtcataaccgggtgtcaaccttgaagtatgccgttccgttctccgaagagttgctcctgtggtctgaccaagcgcagttcgttctgacagcctctggtatcctttcgagtcgctccgtagagttgaaccttacgactcagttcgacgtgcaagacagggcgcgaccacacggtgtaggacgtaatgtatactttgcgtcacctcgcgcttccttcacatccattaaccgatactatgcggtacaggatgtaagctctgtgaagaacgcagaggacatgacggctcacgttccgaactacataccgaatggagtattcagtatatcaggtacaaccgctgagaacttcgcggctatccttacgagcggagcaccgaaccgagtgtatatctacaagttcctctacattgacgaggaaatcaggcagcagtcatggtctcattgggactttggagacaacgttacggtgtttgcagcacaggtgataaactcaacaatgactgtattgatgggtaatgaacatgctgtgtggatgggacgcttacacttcacgaagaactccatagacattccgggagagccttacagattatacatcgacgctaagaggaaatacaccatccctgccgggacgtataatgatgatacctaccagacgtccataagcctcgcaacgatttatgggatgaacttcactaagggtagagtgtctgtggtcttccctgatggaaagattatagaggtagaccagccaatcaacggttggagcagtgacccggtgttacgacttgatgggaaccaagagggccaagtggtttacattgggttcaacatcccgttcacttatacgttctcgaagttcctcattaagaagactgctgaggatggctcgacggctaccgaggatattggtcgcttacagcttcggcgtgcgtgggtaaactatgaggactctggagcgttcaccatccgtgtgaacaacctgtcccgtgagttcatctacacaatggctggtgctcgcttgggttctgacaatctgcgtgttggtaggtcgaacattggcacagggcagtaccgcttcccggtggtgggtaatgcgcagacgaacctagtaaccatcgagtccgatgcgtctaccccattgaacatcattgggtgcggctgggaggggaactaccttcgtcgttcttcgggcatttaacttaaagtctccctgtggtgaattaaccctcactaaagggagacactaatagatacgaggggttaaagcattatgtacattacaaagtgtttacaaatccatgccgacaactttcagccgtccatagaggacattctggaagctgaggcactaagtgttgaaccaaaggtaattcctgatgagaacactgtggcaatgcttagcgataacgctgtgttggcaattggtggcaatcagggagaccgagtgtggttccttacctctaagtacgtcccactgtttaccttgaaggagcgattagagtttcgtagacttatcattgaatatcgtgatatgatgctgagccagtacgagtccatttggaatttcgtttgggtaggcaataagtcacacattcgtttcctaaaaactatcggcgctgtgttccacaatgaatacacaggagatggtcaatttcaactattcacaatcacaaggaggaacccttgaagaagtgcagcaagtgctacgaattgaaggagctaaccgagttctataaggacaagaccaaacctgacggcctgcggtgttcctgcaaggcttgccaaggtgagaccaagaagaagtcctatcgagtcatcaaggatagaaaccttcgtcgtctatacggaatcggtctgaatgagtatgaagaaatgctcactaagcagaatggcgtctgtgcaatctgcggtattaccgagagagttgagggacgggcgttagcggttgaccattgccacgccactggcgctgttcgtggtctcctttgctctgcctgtaacatcgctcttgggaaattcgaggacgacaccgagcgtcttcggaaggcaatcacttacctaagaggagaataattatgtgctggatggcagcgattcctatcgccatgatgggtgcccaagctctgagtagccagaacagtgctgacaaggcgcgagtggcacagaccgaagctggacgccgacaggcaatggagatggtcaaagagatgaatatccagaatgcaaacgcctcgctggaacaacgggacgcccttgaagctgcatcctctgagttgactacacgtaacatgcagaaggtacaggctatgggaaccatccgtgcagctattggcgagggtatgctcgaaggtgagtccatgaagcgcatcaagcgtatcgaagagggcaactacattcgggaggcaaatagtgtcaccgagaattaccgccgagactacgcgagtatctttgcgcaacagttgggacgcactcagtccacagcaagtcaagtcgatgcaatgtacaagagcgaggccaaaggtaagtctggtctgatgcgtgtactagaccctctgtccattatgggtcaggaagctgcaagtcaatatgcggctggtggatttgacaagaaaggtggaaaccaagcagcacctatcagtgccgccaaaggaaccaagaccgggaggtaataatggctagtaaactaaatagtgttttaggcaacatggcgactcccggtatggaacgactccggggcgtcaaaggtatggactaccgggcagcaaccattcaggctgaacaaccaagagcgagtcttctggactccattggtcgattcgctaaggctggtgccgatatgtacatggcgaaagatgaacgtgataagcaacgagccgatgagcgctccaacgagattatccgtaagctgacccctgagcagcgccgacaggctatccagaatggtacattactgtatcaggatgacccatacgcaatgcaagccttgaagttcaagaccgggcgtaacgctgcgttcctcgttgacgatgaggttcaacaggccatcaaagagggtcgcttccgtactcgtgaagagatggagcagtatcgtcactcacgcctacaggagcactctaagtcgttcgctgagcagttcggcatcaatgagatggacgaggagtaccagaagggtttcaatgcgaacatcaccgagcgtaacattgctctgtatggggcacatgataccttcctgagcgaccaagcgcagaagggtgcaatcctgaactcgaaggtcgaactcaatggtgtcctgtctgaccctgacctactgtctcgtcctgagtccggtgagttcttccagaactacattgacaactccttggtgacagggatgaccgacaatcaggctcaacaggttatctcttcgtccctgaatgacgtgctacagcgtccgggtggtgccgccttcttgcagaacattgagaaccgcaaggtaactctaaatggcgctacgactacctatcgtgaactgatgggtgatgaacagtggcaggcaatgatgattaaggcgcagcacactcaattccagaacaacgcgaagttaaccgagaagttccgactggatattaactcagcgcttaaccaagcggacactggtaaaggctgggagactcttcaaggcatcaaggccgaactcgataagattcagccgggtgaagagatgactcctgagcgcgagtggttgatttccgcacaggaacagatgcgcacacgcttcaagcaggaacaggctgagacagccaagctgatggacaagcaacagaagaccctgaataaatctttggtcatcgacaagcagttccagaagcgcctcaacggtgaatacgtctcgaccaactataaggacatgccgacaaacgagaacactggtgagtttacacatagtgatatggtcaactacgcgaaccagaagttagcggaaatcgaagcgatggacttaacgccagaacagaaagacaaactgaaattggactacctacgggcagactccaaggagggtgctttccgtgcagcgtttggtgagatgattactgacgcaggtaacgagtggcaggctgctgtgattaacggtaagatgcctgagagcacaccagcgatggacaaccttcgtcgcatccgcaacactgaccctgacctgattgcctctctgtatcctgacaaggccgagttgttcctgactatggacatgttggataaccaaggtatcgacacgcaggttcttatcgacgctgataagtcccgcgcacagaagaccaaggagatgcagtttgaggatgaccgagcgtgggcagccatgatgaatgattctaagaatccgcaaatcaagtacatgccttcgagtgtccaagggtatgcccggaagatttatgattccgtgaagtaccggacagggaaccctgacatggcaaccgaacaggtggcaaagttcctctctgattcaacgactacgttctccagtagtgacgttgatggtgatacctatggtgtcctgcctaagaacatcctaactgtttctgatgaccctaagtcgtgggagcaagggaagaacattctggacgaagccattaagagaatcacagaggctaacccttggattaccaacaagcaactgacggtgtaccagcagggggatgcaatctacctgatggacacaaccgggcaggttcgcactcggttcgaccaagagttactcaaacgcgaataccagcgcaccgcacagatgcaagctgaggcagcacgagagaaagccctcaaagaggccaacaagcgtgccccaatcagtcgtgtaccacaggcgcgtgaagaagcgcgtaaacgtgtccaagagaaacggaagaagactccgaagtacatctatggacgtaaagaagactaacagtgataaggaggctccatgagctacgataagaacaaacctagcgagttcgatgggttatttcaaaaagcggcagacaaacatggggtctcctatgacctgcttcgtaaattagcattcaatgagtccagtttcaatccaaaggctaaatccccaactggcccgaagggtctcatgcagttcaccaagggtacagccactgcgcttggcctcaaggtcactgatgcagacgatgatgaccgttacaacccggagttggctgtggatgcagccgctcgtcacctgagtgacctcattcgtaaatacgatggggatgaacttaaggcagccctagcgtacaaccaaggcgagggaagaaatggtgcccctcagatgcaggcctacgacaaaggtgattgggcttcaatctctgaggaaggtcgtaactatatgcgtaacctgatggacgtagctaacagtcctcgcaagggggacttggaggcgttcggcggtatcaccccaaaggctaagggcattccatcgggggatgcatttgctggaatcgggaagaagcagacggtaggcactgacctgccggagtcaactggattcaaagtggagggtaaagagcagaaagcacctaacgtgccttatgctaaggacttttgggaaaagactggaacaacgctggacgagtttaacgcacgttcgaccttcttcggaattggtgatgcaacaagtgcggagcttcacaactctgtacttggagttgctttccgtgcagcccgtagcgatgatggttttgatttgttcaaggacaccattacgcctacccgttggaatagccacacttggactccagaagagctagagcgaatccgtaaggaagtgaagaacccctcgtacatcaacgtagtcactggaggctctcctgagaatctggacgccctcattaagatggcgaatgacaactacgagatggacgcgcggtctgccgatgctggtgttggtgccaaacttactgctggtatcgtcggcgctggtgtagacccgctgagctatgttccgctagtaggtgtagccgggaagggtctcaaggtgattaacaaggcgttcgtcgttggcactcaaagcgctggcctagcgattgcctctgagggtatccgtacatcaatagctggtggagaggcccactatgctgatgccgctttaggtggtctcttgtttggtgctggtatgagtgcaatcagtgatgctatcgctgctggtgttcgccgttcccgtgggactgaggttgttaatgacttcgctcctatggcacaccgcttggaagcccgtgagaccgctctgaactctggtggtgaagacctgacccgtatgcccagcgagaaccgtgtgttcgaccgtgagcacgctggcgttgagtattcacctctggagaccgagccgggtgccgttgtgttgcctcaaggtcaaatccttagcgacaccaacccgctgaaccctcagactcttagcgagttcgaggcggtcaaccctgagcgtgccgctcgtggtatctctttgggtggtttcactgagattggcttgaagactctccgctctgagaacccgactgttcgctccattgcgagtgaccttgtgcgttctcctactggcatggagtcagggacttctggtaagttcggtgcgactgcttctgacattaaggagcggctccacgcaaacaatcagcgaacctacaatcagctttatgatgctgtgcgaactgcaatgaaagaccctgagttctccacgggtggggccacaatgagccgtaaggaaatccgacaggaaatctacaagcgtgcagccttagcgattgagcgacctgagcttcaagcaaacctgacaaagggtgagcggaacgtgatgaacatcctcaagcaacactttgacctcaaacgtgaaatcatggagaacccgtcaatcttcggtaacaccaaggcggtcagtatcttccccggctcacgccacaagggaacctacgtgccaaacgtgtacgacagagcaatcaagattgagatggttaaacgctacgataatgatggcttgcagcgagcaatcgcggagtcatggttgacgtcttatcgcgcacgtccagaagtcaaggcgagggtcgatgagtatctgatggaacttaatggtctcaaatccgtgcatgaagttacgcctgagatggtgcagaagcacgcgatggataaggcgtatggtatttcacacactgaccagttctctgcctcctctgtcattgaggataacattgagggtctggtgggtatcgagaacaacaacttcttggaagcccgtaacatgttcgacagtgacatggcggtgactctccctgatggtaacacgttctctgtgaatgacctacgtacctatgacatggctgaaattctgcctgcatacgaccgacgagtagatggggatgttgcaatcatgggcggttccggtaagactaccaaagacctgaaagatgaaatcatggcgctggataagcagtcagagggtaacggcacactgaaaggtgaagtggaagctctcaaggataccgtgaagattctaactggtcgtgctcgtaggaacccggaaggcgcttggggtacagccctgcgctccgtcaatgacctcacgttctttgctaagaacgcctatatgggcgcacagaacgttactgaaatcgctggtatgctggcgaaaggtaacgtctctgcgataactcacggaatacctatgattaacgattgggtcaaccgtggtaaaccgcttcgtgcctctgagattaaggagctacacgggatggtgttcggtaaggaactagaccaac

SEQ ID NO: 6 - protein, 553 aa, synthetic construct
  source 1..553: note = T7p52_Tail fiber protein of T7 phage
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIASDGGWLRFQIHSNGLGFKNIADSRSVPNAIMVENE

SEQ ID NO: 7 - protein, 558 aa, synthetic construct
  source 1..558: note = T3p48_Tail fiber protein of T3 phage
MANVIKTVLTYQLDGSNRDFNIPFEYLARKFVVVTLIGVDRKVLTINADYRFATRTTISLTKAWGPADGYTTIELRRVTSTTDRLVDFTDGSILRAYDLNVAQIQTMHVAEEARDLTADTIGVNNDGHLDARGRRIVNLANAVDDRDAVPFGQLKTMNQNSWQARNEALQFRNEAETFRNQTEVFKNESGTNATNTKQWRDEANGSRDEAEQFKNTAGQYATSAGNSATAAHQSEVNAENSATAAANSANLAEQHADRAEREADKLGNFNGLAGAIDRVDGTNVYWKGGIHANGRLYLTSDGFDCGQYQQFFGGSAGRYSVMEWGDENGWLMHVQRREWTTAIGDNIQLVVNGHIIAQGGDMTGPLKLQNGHALYLESASDKAQYILSKDGNRNNWYIGRGSDNNNDCTFHSYVYGTNLTLKPDYAVVNKRFHVGQAVVATDGNIQGTKWGGKWLDAYLNDTYVKKTMAWTQVWAAASGSYMGGGSQTDTLPQDLRFRNIWIKTRNNYWNFFRTGPDGIYFLSAEGGWLKFQIHSNGRVFKNIADRDAPPTAIAVEDV

SEQ ID NO: 8 - protein, 599 aa, synthetic construct
  source 1..599: note = Bas65_p50_Tail fiber protein of Bas phage
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTADTIGVNNDGHLDARGRRIVNLANAVDDRDAVPFGQLKTMNQNSWQARNEALQFRNEAETFRNQAEGFKTESGANATNTKQWRDDAKGFRDEAEQFKNTAGQYATSAGNSATAAHQSEVNAENSATASANSAHSAEQQADRAKSEADKLANNNAFAGTLHSITPDNPAVMGSGTVTFKSSYAVNAPILRATKGGMSLGSDRVDGDGTSTFINFYKDSRNTVGSIQVGDDGTFTINSNGWTKLAGSVTQVNQLRSGYSEVTVNNNNVAVHARATNLSWDDGQTNAVYNRYTIAGRGSSVEGVDGAIMDFGIREIVGNRYEAYMWVAGFGRSAVWTFGHTGTFTSPADIVANGSLVAGNSLRIGTCTHASDGNIFGSIWGNDWLSNYLNRSFKRKTKEWTSVWNGRIGAGGVAWLTQDVRFRTIWIRTEARPSTWTPIQLGPDGMYMARPDPGWIRFTLANNGTSITNNQDDNSVPIEIAVENE

SEQ ID NO: 9 - protein, 645 aa, synthetic construct
  source 1..645: note = phiYe_p51_Tail fiber protein of phiYeO3-12 phage
MATTIKTVMTYPLDGSTTDFNIPFEYLARKFVRVTLIGVDRKELILNQDYRFATKTTISTTRALGPADGYTLIEIRRFTSATDRLVDFTDGSILRAYDLNISQVQTLHVAEEARDLTADTIGVNNDGNLDARGRRIVNVADAQDVGDAINLGQIQRWNDSALNSANRAKQEADRATARANDANNSANASASSASSSAGSAELAKRWATSDTVVESDLESSRTYALHSMLYRNETKDSADRAAVSETNAKASEGGAANSAAAAKVSETNAKASEERAITEASKLGNMNDFAAAIESVTGNDVKMKGAVSSPGNITGGGLVSTGAASIQKGALVGEDLIVGRDITAKQDMYSQRNIAVAGVTYAQGGIEQTLATNIYNKLYRLHINSNPQHVGQRQGLHIGWNESGSGESNFITNRGAGSGGFVFRTVNAENSVETGRVDITGGGVIYANHLQVRSGARIEGNNNIVGQNLYAGMGSTMFEGNGNLTGGIWAQWGNLWSGLNNNSLFAKPPGGVQLFTARGGYYLEGRVDGTAVGFRWFQSDRRLKEDIKVVRSADDMLNIIRSYIPVSYKYKDASYTDNRGRTNTIEGKRSRAGFITQDLIRLWPEAVDVMSDGMQSPDPNQIIGGLMLLVKNLDARIQELEKDKT

SEQ ID NO: 10 - protein, 553 aa, synthetic construct
  source 1..553: note = T7p52(W523A)_T7p52 protein with point mutation W523A
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIASDGGALRFQIHSNGLGFKNIADSRSVPNAIMVENE

SEQ ID NO: 11 - protein, 553 aa, synthetic construct
  source 1..553: note = T7p52(G521A)_T7p52 protein with point mutation G521A
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIASDAGWLRFQIHSNGLGFKNIADSRSVPNAIMVENE

SEQ ID NO: 12 - protein, 553 aa, synthetic construct
  source 1..553: note = T7p52(S519A)_T7p52 protein with point mutation S519A
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIAADGGWLRFQIHSNGLGFKNIADSRSVPNAIMVENE

SEQ ID NO: 13 - protein, 553 aa, synthetic construct
  source 1..553: note = T7p52(I517A)_T7p52 protein with point mutation I517A
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFAASDGGWLRFQIHSNGLGFKNIADSRSVPNAIMVENE

SEQ ID NO: 14 - protein, 553 aa, synthetic construct
  source 1..553: note = T7p52(S543A)_T7p52 protein with point mutation S543A
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIASDGGWLRFQIHSNGLGFKNIADSRAVPNAIMVENE

SEQ ID NO: 15 - protein, 553 aa, synthetic construct
  source 1..553: note = T7p52(R542A)_T7p52 protein with point mutation R542A
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIASDGGWLRFQIHSNGLGFKNIADSASVPNAIMVENE

SEQ ID NO: 16 - protein, 551 aa, synthetic construct
  source 1..551: note = T7p52(Loop-phiYe_p51)_T7p52 protein with exchange of 2 binding Loops with for the analogous region of phiYe_p51
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIPVSYWLRFQIHSNGLGFKNIADSLIRLAIMVENE

SEQ ID NO: 17 - protein, 553 aa, synthetic construct
  source 1..553: note = T7p52(Loop-T3p48)_T7p52 protein with exchange of 2 binding Loops with for the analogous region of T3p48
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFISAEGGWLRFQIHSNGLGFKNIADRDAPPTAIMVENE

SEQ ID NO: 18 - protein, 604 aa, synthetic construct
  source 1..604: note = T7p52(Tip-phiYe_p51)_Fusion protein of N-terminal part T7p52[1-466] with C-terminal binding domain (137 AA) from phiYe_p51
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKPPGGVQLFTARGGYYLEGRVDGTAVGFRWFQSDRRLKEDIKVVRSADDMLNIIRSYIPVSYKYKDASYTDNRGRTNTIEGKRSRAGFITQDLIRLWPEAVDVMSDGMQSPDPNQIIGGLMLLVKNLDARIQELEKDKT